(19) United States
(12) Patent Application Publication
(10) Pub. No.: US 2022/0017614 A1
(43) Pub. Date: Jan. 20, 2022

(54) Anti-IL6 Agent for Treating Coronavirus Infection

(71) Applicant: Janssen Biotech, Inc., Horsham, PA (US)

(72) Inventors: Katia Boven, Titusville, NJ (US); Jason Chien, Seattle, WA (US); Magda Opsomer, Sint-Pieters-Leeuw (BE); Brian Woodfall, San Francisco, CA (US)

(21) Appl. No.: 17/217,393

(22) Filed: Mar. 30, 2021

Related U.S. Application Data
(60) Provisional application No. 63/012,758, filed on Apr. 20, 2020.
(60) Provisional application No. 63/002,880, filed on Mar. 31, 2020.

(51) Int. Cl. C07K 16/24

(52) U.S. Cl. CPC C07K 16/248; A61K 2039/545; C07K 2317/21; C07K 2317/76

(57) ABSTRACT

An anti-IL6 agent that blocks binding of IL-6 to IL-6 receptor, for example, an anti-IL6 antibody comprising a heavy chain variable region of SEQ ID NO:99 and a light chain variable region of SEQ ID NO:97, is useful in the treatment or prevention of infection of a human with SARS-CoV-1 and/or SARS-CoV-2.

### CROSS-REFERENCE TO RELATED APPLICATIONS

This application claims the benefit of U.S. Provisional Application Ser. No. 63/002,880, filed 31 Mar. 2020, and U.S. Provisional Application Ser. No. 63/012,758, filed 20 Apr. 2020. The entire contents of the aforementioned applications are incorporated herein by reference in their entireties.

### REFERENCE TO SEQUENCE LISTING SUBMITTED ELECTRONICALLY

This application contains a sequence listing, which is submitted electronically via EFS-Web as an ASCII formatted sequence listing with a file name “JBI6281USNP1CORRECTEDSEQLIST.txt”, creation date of 9 Sep. 2021 and having a size of 44 kb. The sequence listing submitted via EFS-Web is part of the specification and is herein incorporated by reference in its entirety.

### INTRODUCTION

The invention relates to anti-interleukin 6 (IL6 or IL-6) agents, for example, an anti-IL6 antibody, for the treatment or prevention of infection of a human with SARS-CoV-1 and/or SARS-CoV-2. The invention also relates to the use of anti-IL6 agents in the manufacture of a medicament for the treatment or prevention of infection of a human with SARS-CoV-1 and/or SARS-CoV-2. The invention further relates to a method of using anti-IL6 agents to treat or prevent infection of a human with SARS-CoV-1 and/or SARS-CoV-2, the method comprising administering a therapeutically effective amount of an anti-IL6 agent to said human.

### BACKGROUND

Coronaviruses cause disease in a number of mammals and birds. Coronaviruses are enveloped viruses having positive-sense single-stranded RNA genomes and are classified as members of the Orthocoronavirinae subfamily, which includes the Alphacoronavirus, Betacoronavirus, Gammacoronavirus and Deltacoronavirus genera. In humans, they have been responsible for important recent outbreaks of infectious diseases, including the Severe Acute Respiratory Syndrome (SARS) outbreak of 2002-2003, the Middle East respiratory syndrome (MERS) outbreaks occurring since 2012, and the coronavirus disease 2019 (COVID-19) pandemic of 2019-2020. SARS, MERS and COVID-19 are all caused by novel coronaviruses (SARS-CoV-1, MERS-CoV and SARS-CoV-2, respectively). The SARS pandemic developed rapidly with the consequence that treatments lacked randomised placebo-controlled clinical trials. Despite this, a combination of ribavirin and corticosteroids was used in Hong Kong, Canada and elsewhere, as well as a pulse methylprednisolone therapy dosed at 250 to 500 mg/day for 3 to 6 days in critically ill patients infected with the virus. There are currently no approved vaccines or antiviral drugs for patients infected with MERS-CoV or SARS-CoV-2.

SARS-CoV-2, the causative agent of COVID-19, is an enveloped, positive-sense, single-stranded RNA betacoronavirus. It was first identified following reports of a cluster of acute respiratory illness cases in Wuhan, Hubei Province, China in December 2019. Epidemiological investigations indicated that the majority of early cases were linked to a seafood market, with patients infected through zoonotic or environmental exposure, followed by the subsequent spread of infection by human-to-human transmission among close contacts. Genomic sequencing was performed on bronchoalveolar lavage fluid sample collected from patients with viral pneumonia admitted to hospitals in Wuhan, which identified a novel RNA virus from the family Coronaviridae. Phylogenetic analysis of the complete viral genome revealed that the virus, SARS-CoV-2, is part of the subgenus Sarbecovirus of the genus Betacoronavirus, and is most closely related (approximately 88% identity) to a group of SARS-like coronaviruses previously sampled from bats in China.

Since the novel SARS-CoV-2 virus was observed in humans in late 2019, hundreds of thousands have been infected and thousands have died as a result of the associated disease, termed COVID-19. Symptoms of infection may appear from 2 to 14 days following exposure, with the spectrum of illnesses ranging from mild symptoms to severe illness or death. Severe clinical presentations have been reported for as many as 20-25% of laboratory-confirmed cases. In a study of 99 patients in a single center in Wuhan with SARS-CoV-2 infection confirmed by real-time reverse-transcriptase polymerase chain reaction (RT-PCR), the most commonly reported clinical manifestations were fever (83%), cough (82%), shortness of breath (31%), and muscle ache (11%). In chest x-rays and CT scans, 75% of patients showed bilateral pneumonia and 14% of patients showed multiple mottling and ground-glass opacities. In a further study of 138 patients with novel coronavirus-induced pneumonia in a single center in Wuhan, common symptoms included fever (98.6%), fatigue (69.6%), and dry cough (59.4%). Lymphopenia occurred in 70.3% of patients, and chest CT scans showed bilateral patchy shadows or ground-glass opacities in the lungs of all patients. Thirty-six patients (26%) were transferred to the ICU because of complications, including acute respiratory distress syndrome, arrhythmia, and shock. Broadly, similar findings were noted in other case studies, e.g., in the Seattle region in the US. At present, it appears that individuals aged 65 years or older, especially those with comorbid diseases, are subject to the highest incidence of morbidity and mortality. In contrast, a study of 2,143 children aged <18 years in China with laboratory confirmed (34.1% of cases) or suspected (65.9% of cases) COVID-19 indicated that the clinical manifestations of the disease may be less severe in children than adults, with approximately 94% of cases being asymptomatic, mild, or moderate. However, young children, particularly infants, were susceptible to severe disease, with the highest proportion of severe and critical cases by age group reported for children aged <1 year (10.6% of cases) and 1-5 years (7.3% of cases). Also, new evidence has emerged from China indicating that a large number of infections do not result in symptoms.

Current management of COVID-19 is supportive without a current established treatment, and respiratory failure from acute respiratory distress syndrome (ARDS) is the leading cause of mortality. Many trials (e.g., with antivirals) are currently ongoing. While an understanding of the epidemiology and clinical spectrum of COVID-19 is still evolving during the ongoing pandemic, the current knowledge of the disease burden highlights the urgent medical need to develop a treatment.

Learnings from SARS-CoV, initial COVID-19 data and preclinical mouse model data indicate that IL-6 might be a key driver of the acute lung injury (ALI) and ARDS observed in COVID-19. More severe COVID-19 patients from an initial study in China had elevated levels of IL-6. Tocilizumab (Actemra/roActemra®) is a human mAb that binds the IL-6 receptor (IL-6R) and blocks the IL-6 pathway. It is approved to treat cytokine release syndrome (CRS) due to chimeric antigen receptor T (CAR-T) cell therapy and has shown anecdotal preliminary benefit in treating ARDS in patients with COVID-19. Noncontrolled, interim data from patients being treated with another IL-6R antibody sarilumab also provide some early reports of possible benefit to patients.

At the time of drafting, the death toll is over 165,000. SARS-CoV-2 and coronaviruses more generally lack effective treatment, leading to a large unmet medical need.

Interleukin-6 (IL-6) is a pro-inflammatory cytokine that is produced by many different cell types. In vivo, stimulated monocytes, fibroblasts, and endothelial cells represent the main sources of IL-6. Other cells such as macrophages, T and B lymphocytes, granulocytes, keratinocytes, mast cells, osteoblasts, chrondrocytes, glial cells, and smooth muscle cells also produce IL-6 after stimulation (Kishimoto, T., Blood 74:1-10 (1989) and Kurihara, N. et al., J. Immunology 144:4226-4230 (1990)). Several tumor cells also produce IL-6 (Smith, P. C. et al. Cytokine and Growth Factor Reviews 12:33-40 (2001)); IL-6 was indicated to be a prognostic factor for prostate cancer progression (Nakashima, J. et al. Clinical Cancer Research 6:2702-2706 (2000)). IL-6 production can be regulated by IL-6 itself and depending upon cell type, IL-6 can stimulate or inhibit its own synthesis.

IL-6 can bind to the IL-6 receptor expressed on mitogen-activated B cells, T cells, peripheral monocytes, and certain tumors (Ishimi, Y. et al., J. Immunology 145:3297-3303 (1990)). The IL-6 receptor has at least two different components and is composed of an alpha chain called gp80 that is responsible for IL-6 binding and a beta chain designated gp130 that is needed for signal transduction (Adebanjo, O. et al., J. Cell Biology 142:1347-1356 (1998) and Poli, V. et al., EMBO 13:1189-1196 (1994)). The cytokine family which includes IL-6, LIF, Oncostatin M, IL-11, CNTF, and CT-1 all signal through gp130 after binding to their cognate receptors. In addition, all members of the IL-6 cytokine family can induce hepatic expression of acute phase proteins (Bellido, T. et al., J. Clin. Investigation 97:431-437 (1996)).

There are at least two major biological functions of IL-6: mediation of acute phase proteins and acting as a differentiation and activation factor (Avvisti, G. et al., Baillieres Clinical Hematology 8:815-829 (1995) and Poli, V. et al., EMBO 13:1189-1196 (1994)). Acute phase proteins are known to regulate immune responses, mediate inflammation, and play a role in tissue remodeling. As a differentiation and activation factor, IL-6 induces B cells to differentiate and secrete antibody, it induces T cells to differentiate into cytotoxic T cells, activates cell signaling factors, and promotes hematopoiesis (Ishimi, Y. et al., J. Immunology 145:3297-3303 (1990)). IL-6 is prominently involved in many critical bodily functions and processes. As a result, physiological processes including bone metabolism, neoplastic transformation, and immune and inflammatory responses can be enhanced, suppressed, or prevented by manipulation of the biological activity of IL-6 in vivo by means of an antibody (Adebanjo, O. et al., J. Cell Biology 142:1347-1356 (1998)).

Sirukumab is a human anti-IL-6 IgG1K mAb that binds to human IL-6 with high affinity and specificity. The high affinity binding of sirukumab to IL-6 (KD=0.175 pM) prevents the association of IL-6 with the IL-6R, thereby blocking receptor signaling and biological activities attributed to IL-6 both in vitro and in vivo. In vitro bioassays show that sirukumab neutralizes both cis- and trans-signaling of human IL-6 (mediated via cell surface IL-6R and soluble IL-6R, respectively) in a concentration-dependent manner. Sirukumab also blocks human IL-6-induced expression of the acute phase proteins haptoglobin and serum amyloid A (SAA) in mice; this effect is sirukumab-specific and dose-dependent.

Current management of COVID-19 is supportive, and respiratory failure from acute respiratory distress syndrome (ARDS) is the leading cause of mortality. While the understanding of the epidemiology and clinical spectrum of COVID-19 is still evolving during the ongoing pandemic, the current knowledge of the disease burden highlights the urgent medical need to develop a treatment.

### BRIEF DESCRIPTION OF THE DRAWINGS

FIG. 1 shows a schema of the sirukumab COVID-19 clinical study design.

### SUMMARY OF THE INVENTION

The present invention relates to anti-IL6 agents comprising molecules that bind to IL6 and block or inhibit an IL6 activity, such as binding of IL6 to IL6 Receptor (IL6R), for use in the treatment or prevention of infection of a human with SARS-CoV-1 and/or SARS-CoV-2 (in particular, COVID-19). The present invention further relates to anti-IL6 agents comprising molecules that bind to IL6 and block or inhibit an IL6 activity, such as binding of IL6 to IL6 Receptor (IL6R), for use in the treatment or prevention of infection of a human with SARS-CoV-1 and/or SARS-CoV-2 (in particular, COVID-19). In an embodiment, the anti-IL6 agent is an anti-IL6 antibody or an isolated anti-IL6 antibody. In another aspect, the invention relates to the use of an anti-IL6 agent in the manufacture of a medicament for the treatment or prevention of infection of a human with SARS-CoV-1 and/or SARS-CoV-2 (e.g., COVID-19). In such embodiments, references to the medicament being administered (e.g., “wherein the medicament is administered”) include the medicament being prepared for administration (and thus may be amended to “wherein the medicament is prepared for administration”) with the features in question. References to the anti-IL6 agent being administered (“anti-IL6 agent is administered”) are referring to the anti-IL6 agent within the medicament that is being administered to the human.

In another aspect, the invention relates to a method of using an anti-IL6 agent to treat or prevent infection of a human with SARS-CoV-1 and/or SARS-CoV-2, the method comprising administering a therapeutically effective amount of an anti-IL6 agent to the human.

In another aspect, the invention relates to an anti-IL6 agent in a package together with instructions for its use in the treatment or prevention of infection of a human with SARS-CoV-1 and/or SARS-CoV-2.

In another aspect, the invention relates to the inhibition of infection of a human cell with SARS-CoV-1 and/or SARS-CoV-2 by treating the cell with an anti-IL6 agent.

In another aspect, the invention relates to the inhibition of one or more viral activities in a human cell infected with SARS-CoV-1 and/or SARS-CoV-2 by treating the cell with an anti-IL6 agent.

In an embodiment, the isolated antibody or an antigen-binding fragment thereof comprises a heavy chain variable region and a light chain variable region having complementarity determining regions (CDRs): i) CDRH1 amino acid sequence of SEQ ID NO: 135; ii) CDRH2 amino acid sequence of SEQ ID NO: 136; iii) CDRH3 amino acid sequence of SEQ ID NO: 137; iv) CDRL1 amino acid sequence of SEQ ID NO: 132; v) CDRL2 amino acid sequence of SEQ ID NO: 133; and vi) CDRL3 amino acid sequence of SEQ ID NO: 134; and wherein X₁ is A or G, X₂ is S or R, X₃ is H, I, S, or Y, X₄ is S or Y, X₅ is S or F, X₆ is F, L, M, or T, X₇ is N or E, X₈ is A or T, X₉ is M, C, S or Q, X₁₀ is Q or C, X₁₁ is T or Q, X₁₂ is F, S, or T, X₁₃ is S or P, X₁₄ is L or M, X₁₅ is A or I, X₁₆ is S or P, X₁₇ is Y or W, X₁₈ is T, E, or Y, X₁₉ is Y or F, X₂₀ is P, S, D, or Y, X₂₁ is V or D, X₂₂ is T or A, X₂₃ is G or P, X₂₄ is S, Y, T, or N, and X₂₅ is Y, T, F, or I. In an alternative embodiment, the antibody comprises the following CDR's: i) CDRH1 amino acid sequence of SEQ ID NO: 39; ii) CDRH2 amino acid sequence of SEQ ID NO: 59; iii) CDRH3 amino acid sequence of SEQ ID NO: 89; iv) CDRL1 amino acid sequence of SEQ ID NO: 3; v) CDRL2 amino acid sequence of SEQ ID NO: 21; and vi) CDRL3 amino acid sequence of SEQ ID NO: 29.

In another embodiment, the isolated antibody or an antigen-binding fragment thereof comprises a heavy chain variable region and a light chain variable region of SEQ ID NO:99 and SEQ ID NO:97, respectively (the antibody sirukumab—also referred to as CNTO136). In another embodiment, the isolated antibody or an antigen-binding fragment thereof comprises a heavy chain variable region and a light chain variable region of SEQ ID NO:139 and SEQ ID NO:140, respectively (the antibody siltuximab). The antibodies described herein are useful in the preparation of a medicament for the treatment of SARS-CoV-1 and/or SARS-CoV-2, wherein the medicament is prepared for administration in indications and dosages defined herein.

In a preferred embodiment, the isolated antibody is administered intravenously (IV) at a dose of 3 to 10 mg per kg weight of the patient, preferably 4 to 8 mg per kg weight of the patient, more preferably 5 mg per kg weight of the patient. Preferably, the isolated antibody is administered intravenously (IV) at a dose of about 5 mg/kg. In a preferred embodiment, the isolated antibody is administered intravenously (IV) at least three or two times a day. Preferably, the isolated antibody is administered intravenously (IV) one time per day as a single IV dose.

In a preferred embodiment, the antibody formulation is 50 to 150 mg/mL of antibody, 30 to 50 mg/mL sorbitol, 0.2 to 0.6 glacial Acetic Acid mg/mL, 0.5 to 1 mg/mL sodium acetate, and 0.2 to 0.6 mg/mL polysorbate. Preferably, the antibody formulation is 100 mg/mL of antibody, 43 mg/mL sorbitol, 0.4 glacial Acetic Acid mg/mL, 0.7 mg/mL sodium acetate, and 0.4 mg/mL polysorbate.

### DETAILED DESCRIPTION OF THE INVENTION

Preferred embodiments of the invention are defined below. These preferred embodiments are applicable to all of the aspects of the invention defined herein.

The antibodies of the invention can bind human IL-6 with a wide range of affinities (K_{D}). In a preferred embodiment, at least one human mAb of the present invention can optionally bind human IL-6 with high affinity. For example, a human or human engineered mAb can bind human IL-6 with a KD equal to or less than about 10⁻⁷ M, such as but not limited to, 0.1-9.9 (or any range or value therein)×10⁻⁷, 10⁻⁸, 10⁻⁹, 10⁻¹⁰, 10⁻¹¹, 10⁻¹², 10⁻¹³, 10⁻¹⁴, 10⁻¹⁵ or any range or value therein, as determined by surface plasmon resonance or the Kinexa method, as practiced by those of skill in the art.

The affinity or avidity of an antibody for an antigen can be determined experimentally using any suitable method. (See, for example, Berzofsky, et al., “Antibody-Antigen Interactions,” In Fundamental Immunology, Paul, W. E., Ed., Raven Press: New York, N.Y. (1984); Kuby, Janis Immunology, W. H. Freeman and Company: New York, N.Y. (1992); and methods described herein). The measured affinity of a particular antibody-antigen interaction can vary if measured under different conditions (e.g., salt concentration, pH). Thus, measurements of affinity and other antigen-binding parameters (e.g., KD, Kon, Koff) are preferably made with standardized solutions of antibody and antigen, and a standardized buffer, such as the buffer described herein.

Preferred anti-IL-6 antibodies of the invention have the sequences shown in Tables 1-9 below. For example, an anti-IL-6 antibody of the invention has one of the light chain CDR sequences shown in Table 1 (i.e., CDRL1, CDRL2, and CDRL3) and one of the heavy chain CDR sequences shown in Table 2 (i.e., CDRH1, CDRH2, and CDRH3). More specifically, an anti-IL-6 antibody (molecule AME-A9) has the CDRL1 of SEQ ID NO:15, CDRL2 of SEQ ID NO:27, CDRL3 of SEQ ID NO:35, CDRH1 of SEQ ID NO:47, CDRH2 of SEQ ID NO:61, CDRH3 of SEQ ID NO:91.

TABLE 1
Light Chain CDRs

| SEQ | CDR | | |
|---|---|---|---|
| ID NO | Name* | Clone | Sequence |
| SEQ ID | CDRL1 | 33 | SASHSVSYMY |
| NO: 1 | | | |
| SEQ ID | CDRL1 | 33 | AGTGCCAGCCATAGTGTAAGTTACATGT |
| NO: 2 | | | AC |
| SEQ ID | CDRL1 | 34 | SASISVSYMY |
| NO: 3 | | | |
| SEQ ID | CDRL1 | 34 | AGTGCCAGCATTAGTGTAAGTTACATGT |
| NO: 4 | | | AC |
| SEQ ID | CDRL1 | 35 | SARSSVSYMY |
| NO: 5 | | | |
| SEQ ID | CDRL1 | 35 | AGTGCCCGGTCAAGTGTAAGTTACATGT |
| NO: 6 | | | AC |
| SEQ ID | CDRL1 | 36 | SASYSVSYMY |
| NO: 7 | | | |
| SEQ ID | CDRL1 | 36 | AGTGCCAGCTATAGTGTAAGTTACATGT |
| NO: 8 | | | AC |
| SEQ ID | CDRL1 | 37 | SASSSVFYMY |
| NO: 9 | | | |
| SEQ ID | CDRL1 | 37 | AGTGCCAGCTCAAGTGTATTTTACATGT |
| NO: 10 | | | AC |
| SEQ ID | CDRL1 | 39 | SGSSYVSYMY |
| NO: 11 | | | |
| SEQ ID | CDRL1 | 39 | AGTGGCAGCTCATATGTAAGTTACATGT |
| NO: 12 | | | AC |
| SEQ ID | CDRL1 | 40 | SALSSVSYMY |
| NO: 13 | | | |
| SEQ ID | CDRL1 | 40 | AGTGCCCTGTCAAGTGTAAGTTACATGT |
| NO: 14 | | | AC |
| SEQ ID | CDRL1 | A9 | SASSSVSYMY |
| NO: 15 | | | |
| SEQ ID | CDRL1 | A9 | AGTGCCAGCTCAAGTGTAAGTTACATGT |
| NO: 16 | | | AC |
| SEQ ID | CDRL2 | 41 | DFSNLAS |
| NO: 17 | | | |
| SEQ ID | CDRL2 | 41 | GACTTTTCCAACCTGGCTTCT |
| NO: 18 | | | |
| SEQ ID | CDRL2 | 43 | DLSNLAS |
| NO: 19 | | | |
| SEQ ID | CDRL2 | 43 | GACCTGTCCAACCTGGCTTCT |
| NO: 20 | | | |
| SEQ ID | CDRL2 | 44 | DMSNLAS |
| NO: 21 | | | |
| SEQ ID | CDRL2 | 44 | GACATGTCCAACCTGGCTTCT |
| NO: 22 | | | |
| SEQ ID | CDRL2 | 46 | DTSNLTS |
| NO: 23 | | | |
| SEQ ID | CDRL2 | 46 | GACACATCCAACCTGACGTCT |
| NO: 24 | | | |
| SEQ ID | CDRL2 | 48 | DTSELAS |
| NO: 25 | | | |
| SEQ ID | CDRL2 | 48 | GACACATCCGAGCTGGCTTCT |
| NO: 26 | | | |
| SEQ ID | CDRL2 | A9 | DTSNLAS |
| NO: 27 | | | |
| SEQ ID | CDRL2 | A9 | GACACATCCAACCTGGCTTCT |
| NO: 28 | | | |
| SEQ ID | CDRL3 | 49 | MQWSGYPYT |
| NO: 29 | | | |
| SEQ ID | CDRL3 | 49 | ATGCAGTGGAGTGGTTACCCATACACG |
| NO: 30 | | | |
| SEQ ID | CDRL3 | 50 | CQWSGYPYT |
| NO: 31 | | | |
| SEQ ID | CDRL3 | 50 | TGTCAGTGGAGTGGTTACCCATACACG |
| NO: 32 | | | |
| SEQ ID | CDRL3 | 52 | SCWSGYPYT |
| NO: 33 | | | |
| SEQ ID | CDRL3 | 52 | TCTGTGTGGAGTGGTTACCCATACACG |
| NO: 34 | | | |
| SEQ ID | CDRL3 | A9 | SQWSGYPYT |
| NO: 35 | | | |
| SEQ ID | CDRL3 | A9 | TCTCAGTGGAGTGGTTACCCATACACG |
| NO: 36 | | | |
| SEQ ID | CDRL3 | Alt. | QQWSGYPYT |
| NO: 138 | | | |

*CDRs were as defined by Kabat with the exception of CDRH1 which is the sum of Kabat and Chothia definitions.

TABLE 2
Heavy Chain CDRs

| SEQ | CDR | | |
|---|---|---|---|
| ID NO | Name* | Clone | Sequence |
| SEQ ID | CDRH1 | 4 | GFTFSSFALS |
| NO: 37 | | | |
| SEQ ID | CDRH1 | 4 | GGATTCACCTTTAGTAGCTTTGCCCTTT |
| NO: 38 | | | CT |
| SEQ ID | CDRH1 | 5 | GFTFSPFAMS |
| NO: 39 | | | |
| SEQ ID | CDRH1 | 5 | GGATTCACCTTTAGTCCTTTTGCCATGT |
| NO: 40 | | | CT |
| SEQ ID | CDRH1 | 6 | GFQFSSFAMS |
| NO: 41 | | | |
| SEQ ID | CDRH1 | 6 | GGATTCCAGTTTAGTAGCTTTGCCATGT |
| NO: 42 | | | CT |
| SEQ ID | CDRH1 | 8 | GFTTSSFAMS |
| NO: 43 | | | |
| SEQ ID | CDRH1 | 8 | GGATTCACCACTAGTAGCTTTGCCATGT |
| NO: 44 | | | CT |
| SEQ ID | CDRH1 | Q + P | GFQFSPFAMS |
| NO: 45 | | | |
| SEQ ID | CDRH1 | Q + P | GGATTCCAGTTTAGTCCTTTTGCCATGT |
| NO: 46 | | | CT |
| SEQ ID | CDRH1 | A9 | GFTFSSFAMS |
| NO: 47 | | | |
| SEQ ID | CDRH1 | A9 | GGATTCACCTTTAGTAGCTTTGCCATGT |
| NO: 48 | | | CT |
| SEQ ID | CDRH2 | 10 | KASSGGSYTYYPDTVTG |
| NO: 49 | | | |
| SEQ ID | CDRH2 | 10 | AAAGCGAGTAGTGGTGGGAGTTACACCT |
| NO: 50 | | | ACTATCCTGACACTGTGACGGGC |
| SEQ ID | CDRH2 | 11 | KISSGGSYEYYPDTVTG |
| NO: 51 | | | |
| SEQ ID | CDRH2 | 11 | AAAATTAGTAGTGGTGGGAGTTACGAGT |
| NO: 52 | | | ACTATCCTGACACTGTGACGGGC |
| SEQ ID | CDRH2 | 12 | KISSGGSYYYYPDTVTG |
| NO: 53 | | | |
| SEQ ID | CDRH2 | 12 | AAAATTAGTAGTGGTGGGAGTTACTATT |
| NO: 54 | | | ACTATCCTGACACTGTGACGGGC |
| SEQ ID | CDRH2 | 14 | KISSGGSWTYYPDTVTG |
| NO: 55 | | | |
| SEQ ID | CDRH2 | 14 | AAAATTAGTAGTGGTGGGAGTTGGACCT |
| NO: 56 | | | ACTATCCTGACACTGTGACGGGC |
| SEQ ID | CDRH2 | 16 | KISPGGSYTYYPDTVTG |
| NO: 57 | | | |
| SEQ ID | CDRH2 | 16 | AAAATTAGTCCGGGTGGGAGTTACACCT |
| NO: 58 | | | ACTATCCTGACACTGTGACGGGC |
| SEQ ID | CDRH2 | P + | KISPGGSWTYYSDTVTG |
| NO: 59 | | W + S | |
| | | (18a, | |
| | | 19a) | |
| SEQ ID | CDRH2 | P + | AAAATTAGTCCGGGTGGGAGTTGGACCT |
| NO: 60 | | W + S | ACTATTCTGACACTGTGACGGGC |
| | | (18a, | |
| | | 19a) | |
| SEQ ID | CDRH2 | A9 | KISSGGSYTYYPDTVTG |
| NO: 61 | | | |
| SEQ ID | CDRH2 | A9 | AAAATTAGTAGTGGTGGGAGTTACACCT |
| NO: 62 | | | ACTATCCTGACACTGTGACGGGC |
| SEQ ID | CDRH2 | Alt. | EISSGGSYTYYPDTVTG |
| NO: 113 | | | |
| SEQ ID | CDRH2 | 17 | KISSGGSYTYFPDTVTG |
| NO: 63 | | | |
| SEQ ID | CDRH2 | 17 | AAAATTAGTAGTGGTGGGAGTTACACCT |
| NO: 64 | | | ACTTTCCTGACACTGTGACGGGC |
| SEQ ID | CDRH2 | 19 | KISSGGSYTYYPDTVAG |
| NO: 65 | | | |
| SEQ ID | CDRH2 | 19 | AAAATTAGTAGTGGTGGGAGTTACACCT |
| NO: 66 | | | ACTATCCTGACACTGTGGCTGGC |
| SEQ ID | CDRH2 | 20 | KISSGGSYTYYDDTVTG |
| NO: 67 | | | |
| SEQ ID | CDRH2 | 20 | AAAATTAGTAGTGGTGGGAGTTACACCT |
| NO: 68 | | | ACTATGATGACACTGTGACGGGC |
| SEQ ID | CDRH2 | 21 | KISSGGSYTYYSDTVTG |
| NO: 69 | | | |
| SEQ ID | CDRH2 | 21 | AAAATTAGTAGTGGTGGGAGTTACACCT |
| NO: 70 | | | ACTATTCTGACACTGTGACGGGC |
| SEQ ID | CDRH2 | 22 | KISSGGSYTYYPDTVTP |
| NO: 71 | | | |
| SEQ ID | CDRH2 | 22 | AAAATTAGTAGTGGTGGGAGTTACACCT |
| NO: 72 | | | ACTATCCTGACACTGTGACGCCG |
| SEQ ID | CDRH2 | 23 | KISSGGSYTYYPDTDTG |
| NO: 73 | | | |
| SEQ ID | CDRH2 | 23 | AAAATTAGTAGTGGTGGGAGTTACACCT |
| NO: 74 | | | ACTATCCTGACACTGATACGGGC |
| SEQ ID | CDRH2 | P + S | KISPGGSYTYYSDTVTG |
| NO: 75 | | (20b, | |
| | | 23a) | |
| SEQ ID | CDRH2 | P + S | AAAATTAGTCCGGGTGGGAGTTACACCT |
| NO: 76 | | (20b, | ACTATTCTGACACTGTGACGGGC |
| | | 23a) | |
| SEQ ID | CDRH2 | P + | KISPGGSWTYYDDTVTG |
| NO: 77 | | W + D | |
| | | (22a) | |
| SEQ ID | CDRH2 | P + | AAAATTAGTCCGGGTGGGAGTTGGACCT |
| NO: 78 | | W + D | ACTATGATGACACTGTGACGGGC |
| | | (22a) | |
| SEQ ID | CDRH3 | 25 | QLWGSYALDY |
| NO: 79 | | | |
| SEQ ID | CDRH3 | 25 | CAGTTATGGGGGTCGTATGCTCTTGACT |
| NO: 80 | | | AC |
| SEQ ID | CDRH3 | 26 | QLWGYYALDT |
| NO: 81 | | | |
| SEQ ID | CDRH3 | 26 | CAGTTATGGGGGTACTATGCTCTTGACA |
| NO: 82 | | | CG |
| SEQ ID | CDRH3 | 29 | QLWGTYALDY |
| NO: 83 | | | |
| SEQ ID | CDRH3 | 29 | CAGTTATGGGGGACTTATGCTCTTGACT |
| NO: 84 | | | AC |
| SEQ ID | CDRH3 | 30 | QLWGNYALDY |
| NO: 85 | | | |
| SEQ ID | CDRH3 | 30 | CAGTTATGGGGGAATTATGCTCTTGACT |
| NO: 86 | | | AC |
| SEQ ID | CDRH3 | 31 | QLWGYYALDF |
| NO: 87 | | | |
| SEQ ID | CDRH3 | 31 | CAGTTATGGGGGTACTATGCTCTTGACT |
| NO: 88 | | | TT |
| SEQ ID | CDRH3 | 32 | QLWGYYALDI |
| NO: 89 | | | |
| SEQ ID | CDRH3 | 32 | CAGTTATGGGGGTACTATGCTCTTGACA |
| NO: 90 | | | TT |
| SEQ ID | CDRH3 | A9 | QLWGYYALDY |
| NO: 91 | | | |
| SEQ ID | CDRH3 | A9 | CAGTTATGGGGGTACTATGCTCTTGACT |
| NO: 92 | | | AC |
| SEQ ID | CDRH3 | Alt. | GLWGYYALDY |
| NO: 114 | | | |

*CDRs were as defined by Kabat with the exception of CDRH1 which is the sum of Kabat and Chothia definitions.

TABLE 3
Mutations from Individual CDR libraries

| Clone | |
|---|---|
| | CDRH1 |
| 4 | M34L |
| 5 | S31P |
| 6 | T28Q |
| 8 | F29T |
| | CDRH2 |
| 10 | I51A |
| 11 | T57E |
| 12 | T57Y |
| 14 | Y56W |
| 16 | S52aP |
| 17 | Y59F |
| 19 | T64A |
| 20 | P60D |
| 21 | P60S |
| 22 | G65P |
| 23 | V63D |
| | CDRH3 |
| 25 | Y99S |
| 26 | Y102T |
| 27 | Y99S |
| 29 | Y99T |
| 30 | Y99N |
| 31 | Y102F |
| 32 | Y102I |
| | CDRL1 |
| 33 | S27H |
| 34 | S27I |
| 35 | S26R |
| 36 | S27Y |
| 37 | S30F |
| 38 | S27I |
| 39 | A25G, S28Y |
| 40 | S26L |
| | CDRL2 |
| 41 | T51F |
| 43 | T51L |
| 44 | T51M |
| 46 | A55T |
| 47 | T51L |
| 48 | N53E |
| | CDRL3 |
| 49 | Q89M |
| 50 | Q89C |
| 52 | Q90C |

TABLE 4
Mutations Included in the Combinatorial Library

| CDRH1 | CDRH2 | CDRH3 | CDRL1 | CDRL2 | CDRL3 |
|---|---|---|---|---|---|
| T28Q | S52aP | Y102F | S27I | T51F | Q89M |
| S31P | Y56W | Y102I | S27Y | T51M | |
| | P60S | | | | |
| | V63D | | | | |

TABLE 5A
Positive Library Clones

| | Light | | | Heavy | | | | | | | | |
|---|---|---|---|---|---|---|---|---|---|---|---|---|
| CDR--> | L1 | L2 | L3 | H1 | | H2 | | | | | H3 | |
| WT--> | | | | | | | | | | | | |
| CNTO328 | S | T | Q | T | S | E | S | Y | P | V | G | Y |
| Clone | 27 | 51 | 89 | 28 | 31 | 50 | 52a | 56 | 60 | 63 | 95 | 102 |
| AME-A9 | | | S | | | K | | | | | Q | |
| AME-16 | | | S | | | K | P | | | | Q | |
| | | | | | | | P | | | | | |
| AME-18a | | F | M | Q | P | K | | W | S | | Q | I |
| AME-19a | I | M | M | | P | K | P | W | S | | Q | I |
| AME-20b | I | M | M | Q | | K | P | | S | | Q | I |
| AME-22a | Y | F | M | Q | p | K | P | W | | D | Q | F |
| AME-23a | Y | M | M | Q | | K | P | | S | | Q | F |

TABLE 5B
Human Engineered Anti-IL-6 Antibody
Clones and Corresponding CDRs

| CDR--> | L1 | L2 | L3 | H1 | H2 | H3 |
|---|---|---|---|---|---|---|
| AME-A9 | SEQ | SEQ | SEQ | SEQ | SEQ | SEQ |
| | ID: 15 | ID: 27 | ID: 35 | ID: 47 | ID: 61 | ID: 91 |
| AME-16 | SEQ | SEQ | SEQ | SEQ | SEQ | SEQ |
| | ID: 15 | ID: 27 | ID: 35 | ID: 47 | ID: 57 | ID: 91 |
| AME-18a | SEQ | SEQ | SEQ | SEQ | SEQ | SEQ |
| | ID: 15 | ID: 17 | ID: 29 | ID: 45 | ID: 59 | ID: 89 |
| AME-19a | SEQ | SEQ | SEQ | SEQ | SEQ | SEQ |
| | ID: 3 | ID: 21 | ID: 29 | ID: 39 | ID: 59 | ID: 89 |
| AME-20b | SEQ | SEQ | SEQ | SEQ | SEQ | SEQ |
| | ID: 3 | ID: 21 | ID: 29 | ID: 41 | ID: 75 | ID: 89 |
| AME-22a | SEQ | SEQ | SEQ | SEQ | SEQ | SEQ |
| | ID: 7 | ID: 17 | ID: 29 | ID: 45 | ID: 77 | ID: 87 |
| AME-23a | SEQ | SEQ | SEQ | SEQ | SEQ | SEQ |
| | ID: 7 | ID: 21 | ID: 29 | ID: 41 | ID: 75 | ID: 87 |

TABLE 6
Variable region sequences of clones

| | | Heavy (H) | |
|---|---|---|---|
| SEQ | | or Light (L) | |
| ID NO | Clone | Chain V Region | Sequence |
| 93 | A9 | L Chain AA | EIVLTQSPATLSLSPGERATLSCSASSSVS |
| | | | YMYWYQQKPGQAPRLLIYDTSNLASGIPAR |
| | | | FSGSGSGTDFTLTISSLEPEDFAVYYCSQW |
| | | | SGYPYTFGGGTKVEIK |
| 94 | | L Chain | GAAATTGTGTTGACACAGTCTCCAGCCACCCTGTCTTTGTCTCCAGGGGA |
| | | Nucleotide | AAGAGCCACCCTCTCCTGCAGTGCCAGCTCAAGTGTAAGTTACATGTACT |
| | | | GGTACCAACAGAAACCTGGCCAGGCTCCCAGGCTCCTCATCTATGACACA |
| | | | TCCAACCTGGCTTCTGGCATCCCAGCCAGGTTCAGTGGCAGTGGGTCTGG |
| | | | GACAGACTTCACTCTCACCATCAGCAGCCTAGAGCCTGAAGATTTTGCAG |
| | | | TTTATTACTGTTCTCAGTGGAGTGGTTACCCATACACGTTCGGCGGAGGG |
| | | | ACCAAGGTGGAGATCAAA |
| 95 | | H Chain AA | EVQLVESGGGLVQPGGSLRLSCAASGFTFS |
| | | | SFAMSWVRQAPGKGLEWVAKISSGGSYTYY |
| | | | PDTVTGRFTISRDNAKNSLYLQMNSLRAED |
| | | | TAVYYCARQLWGYYALDYWGQGTTVTVSS |
| 96 | | H Chain | GAGGTGCAGCTGGTGGAGTCTGGGGGAGGCTTGGTCCAGCCTGGGGGGTC |
| | | Nucleotide | CCTGAGACTCTCCTGTGCAGCCTCTGGATTCACCTTTAGTAGCTTTGCCA |
| | | | TGTCTTGGGTCCGCCAGGCTCCAGGGAAGGGGCTGGAGTGGGTGGCCAAA |
| | | | ATTAGTAGTGGTGGGAGTTACACCTACTATCCTGACACTGTGACGGGCCG |
| | | | ATTCACCATCTCCAGAGACAACGCCAAGAACTCACTGTATCTGCAAATGA |
| | | | ACAGCCTGAGAGCCGAGGACACGGCTGTGTATTACTGTGCGAGACAGTTA |
| | | | TGGGGGTACTATGCTCTTGACTACTGGGGCCAAGGGACCACGGTCACCGT |
| | | | CTCCTCA |
| 97 | 19A | L Chain AA | EIVLTQSPATLSLSPGERATLSCSASISVS |
| | | | YMYWYQQKPGQAPRLLIYDMSNLASGIPAR |
| | | | FSGSGSGTDFTLTISSLEPEDFAVYYCMQW |
| | | | SGYPYTFGGGTKVEIK |
| 98 | | L Chain | GAAATTGTGTTGACACAGTCTCCAGCCACCCTGTCTTTGTCTCCAGGGGA |
| | | Nucleotide | AAGAGCCACCCTCTCCTGCAGTGCCAGCATTAGTGTAAGTTACATGTACT |
| | | | GGTACCAACAGAAACCTGGCCAGGCTCCCAGGCTCCTCATCTATGACATG |
| | | | TCCAACCTGGCTTCTGGCATCCCAGCCAGGTTCAGTGGCAGTGGGTCTGG |
| | | | GACAGACTTCACTCTCACCATCAGCAGCCTAGAGCCTGAAGATTTTGCAG |
| | | | TTTATTACTGTATGCAGTGGAGTGGTTACCCATACACGTTCGGCGGAGGG |
| | | | ACCAAGGTGGAGATCAAA |
| 99 | | H Chain AA | EVQLVESGGGLVQPGGSLRLSCAASGFTFS |
| | | | PFAMSWVRQAPGKGLEWVAKISPGGSWTYY |
| | | | SDTVTGRFTISRDNAKNSLYLQMNSLRAED |
| | | | TAVYYCARQLWGYYALDIWGQGTTVTVSS |
| 100 | | H Chain | GAGGTGCAGCTGGTGGAGTCTGGGGGAGGCTTGGTCCAGCCTGGGGGGTC |
| | | Nucleotide | CCTGAGACTCTCCTGTGCAGCCTCTGGATTCACCTTTAGTCCTTTTGCCA |
| | | | TGTCTTGGGTCCGCCAGGCTCCAGGGAAGGGGCTGGAGTGGGTGGCCAAA |
| | | | ATTAGTCCGGGTGGGAGTTGGACCTACTATTCTGACACTGTGACGGGCCG |
| | | | ATTCACCATCTCCAGAGACAACGCCAAGAACTCACTGTATCTGCAAATGA |
| | | | ACAGCCTGAGAGCCGAGGACACGGCTGTGTATTACTGTGCGAGACAGTTA |
| | | | TGGGGGTACTATGCTCTTGACATTTGGGGCCAAGGGACCACGGTCACCGT |
| | | | CTCCTCA |
| 101 | 23A | L Chain AA | EIVLTQSPATLSLSPGERATLSCSASYSVS |
| | | | YMYWYQQKPGQAPRLLIYDMSNLASGIPAR |
| | | | FSGSGSGTDFTLTISSLEPEDFAVYYCMQW |
| | | | SGYPYTFGGGTKVEIK |
| 102 | | L Chain | GAAATTGTGTTGACACAGTCTCCAGCCACCCTGTCTTTGTCTCCAGGGGA |
| | | Nucleotide | AAGAGCCACCCTCTCCTGCAGTGCCAGCTATAGTGTAAGTTACATGTACT |
| | | | GGTACCAACAGAAACCTGGCCAGGCTCCCAGGCTCCTCATCTATGACATG |
| | | | TCCAACCTGGCTTCTGGCATCCCAGCCAGGTTCAGTGGCAGTGGGTCTGG |
| | | | GACAGACTTCACTCTCACCATCAGCAGCCTAGAGCCTGAAGATTTTGCAG |
| | | | TTTATTACTGTATGCAGTGGAGTGGTTACCCATACACGTTCGGCGGAGGG |
| | | | ACCAAGGTGGAGATCAAA |
| 103 | | H Chain AA | EVQLVESGGGLVQPGGSLRLSCAASGFQFS |
| | | | SFAMSWVRQAPGKGLEWVAKISPGGSYTYY |
| | | | SDTVTGRFTISRDNAKNSLYLQMNSLRAED |
| | | | TAVYYCARQLWGYYALDFWGQGTTVTVSS |
| 104 | | H Chain | GAGGTGCAGCTGGTGGAGTCTGGGGGAGGCTTGGTCCAGCCTGGGGGGTC |
| | | Nucleotide | CCTGAGACTCTCCTGTGCAGCCTCTGGATTCCAGTTTAGTAGCTTTGCCA |
| | | | TGTCTTGGGTCCGCCAGGCTCCAGGGAAGGGGCTGGAGTGGGTGGCCAAA |
| | | | ATTAGTCCGGGTGGGAGTTACACCTACTATTCTGACACTGTGACGGGCCG |
| | | | ATTCACCATCTCCAGAGACAACGCCAAGAACTCACTGTATCTGCAAATGA |
| | | | ACAGCCTGAGAGCCGAGGACACGGCTGTGTATTACTGTGCGAGACAGTTA |
| | | | TGGGGGTACTATGCTCTTGACTTTTGGGGCCAAGGGACCACGGTCACCGT |
| | | | CTCCTCA |
| 116 | AME-16 | L Chain AA | EIVLTQSPATLSLSPGERATLSCSASSSVS |
| | | | YMYWYQQKPGQAPRLLIYDTSNLASGIPAR |
| | | | FSGSGSGTDFTLTISSLEPEDFAVYYCSQW |
| | | | SGYPYTFGGGTKVEIK |
| 117 | | L Chain | ATGGAAGCCCCAGCGCAGCTTCTCTTCCTCCTGCTACTCTGGCTCCCAGA |
| | | Nucleotide | TACCACCGGAGAAATTGTGTTGACACAGTCTCCAGCCACCCTGTCTTTGT |
| | | | CTCCAGGGGAAAGAGCCACCCTCTCCTGCAGTGCCAGCTCAAGTGTAAGT |
| | | | TACATGTACTGGTACCAACAGAAACCTGGCCAGGCTCCCAGGCTCCTCAT |
| | | | CTATGACACATCCAACCTGGCTTCTGGCATCCCAGCCAGGTTCAGTGGCA |
| | | | GTGGGTCTGGGACAGACTTCACTCTCACCATCAGCAGCCTAGAGCCTGAA |
| | | | GATTTTGCAGTTTATTACTGTTCTCAGTGGAGTGGTTACCCATACACGTT |
| | | | CGGCGGAGGGACCAAGGTGGAGATCAAA |
| 118 | | H Chain AA | EVQLVESGGGLVQPGGSLRLSCAASGFTFS |
| | | | SFAMSWVRQAPGKGLEWVAKISPGGSYTYY |
| | | | PDTVTGRFTISRDNAKNSLYLQMNSLRAED |
| | | | TAVYYCARQLWGYYALDYWGQGTTVTVSS |
| 119 | | H Chain | ATGGAGTTTGGCCTGAGCTGGGTTTTCCTTGTTGCTATTTTAGAAGGTGT |
| | | Nucleotide | CCAGTGTGAGGTGCAGCTGGTGGAGTCTGGGGGAGGCTTGGTCCAGCCTG |
| | | | GGGGGTCCCTGAGACTCTCCTGTGCAGCCTCTGGATTCACCTTTAGTAGC |
| | | | TTTGCCATGTCTTGGGTCCGCCAGGCTCCAGGGAAGGGGCTGGAGTGGGT |
| | | | GGCCAAAATTAGTCCCGGTGGGAGTTACACCTACTATCCTGACACTGTGA |
| | | | CGGGCCGATTCACCATCTCCAGAGACAACGCCAAGAACTCACTGTATCTG |
| | | | CAAATGAACAGCCTGAGAGCCGAGGACACGGCTGTGTATTACTGTGCGAG |
| | | | ACAGTTATGGGGGTACTATGCTCTTGACTACTGGGGCCAAGGGACCACGG |
| | | | TCACCGTCTCCTCA |
| 120 | AME-18a | L Chain AA | EIVLTQSPATLSLSPGERATLSCSASSSVS |
| | | | YMYWYQQKPGQAPRLLIYDFSNLASGIPAR |
| | | | FSGSGSGTDFTLTISSLEPEDFAVYYCMQW |
| | | | SGYPYTFGGGTKVEIK |
| 121 | | L Chain | ATGGAAGCCCCAGCGCAGCTTCTCTTCCTCCTGCTACTCTGGCTCCCAGA |
| | | Nucleotide | TACCACCGGAGAAATTGTGTTGACACAGTCTCCAGCCACCCTGTCTTTGT |
| | | | CTCCAGGGGAAAGAGCCACCCTCTCCTGCAGTGCCAGCTCAAGTGTAAGT |
| | | | TACATGTACTGGTACCAACAGAAACCTGGCCAGGCTCCCAGGCTCCTCAT |
| | | | CTATGACTTCTCCAACCTGGCTTCTGGCATCCCAGCCAGGTTCAGTGGCA |
| | | | GTGGGTCTGGGACAGACTTCACTCTCACCATCAGCAGCCTAGAGCCTGAA |
| | | | GATTTTGCAGTTTATTACTGTATGCAGTGGAGTGGTTACCCATACACGTT |
| | | | CGGCGGAGGGACCAAGGTGGAGATCAAA |
| 122 | | H Chain AA | EVQLVESGGGLVQPGGSLRLSCAASGFQFS |
| | | | PFAMSWVRQAPGKGLEWVAKISPGGSWTYY |
| | | | SDTVTGRFTISRDNAKNSLYLQMNSLRAED |
| | | | TAVYYCARQLWGYYALDIWGQGTTVTVSS |
| 123 | | H Chain | ATGGAGTTTGGCCTGAGCTGGGTTTTCCTTGTTGCTATTTTAGAAGGTGT |
| | | Nucleotide | CCAGTGTGAGGTGCAGCTGGTGGAGTCTGGGGGAGGCTTGGTCCAGCCTG |
| | | | GGGGGTCCCTGAGACTCTCCTGTGCAGCCTCTGGATTCCAGTTTAGTCCC |
| | | | TTTGCCATGTCTTGGGTCCGCCAGGCTCCAGGGAAGGGGCTGGAGTGGGT |
| | | | GGCCAAAATTAGTCCCGGTGGGAGTTGGACCTACTATAGCGACACTGTGA |
| | | | CGGGCCGATTCACCATCTCCAGAGACAACGCCAAGAACTCACTGTATCTG |
| | | | CAAATGAACAGCCTGAGAGCCGAGGACACGGCTGTGTATTACTGTGCGAG |
| | | | ACAGTTATGGGGGTACTATGCTCTTGACATTTGGGGCCAAGGGACCACGG |
| | | | TCACCGTCTCCTCA |
| 124 | AME-20b | L Chain AA | EIVLTQSPATLSLSPGERATLSCSASISVS |
| | | | YMYWYQQKPGQAPRLLIYDMSNLASGIPAR |
| | | | FSGSGSGTDFTLTISSLEPEDFAVYYCMQW |
| | | | SGYPYTFGGGTKVEIK |
| 125 | | L Chain | ATGGAAGCCCCAGCGCAGCTTCTCTTCCTCCTGCTACTCTGGCTCCCAGA |
| | | Nucleotide | TACCACCGGAGAAATTGTGTTGACACAGTCTCCAGCCACCCTGTCTTTGT |
| | | | CTCCAGGGGAAAGAGCCACCCTCTCCTGCAGTGCCAGCATTAGTGTAAGT |
| | | | TACATGTACTGGTACCAACAGAAACCTGGCCAGGCTCCCAGGCTCCTCAT |
| | | | CTATGACATGTCCAACCTGGCTTCTGGCATCCCAGCCAGGTTCAGTGGCA |
| | | | GTGGGTCTGGGACAGACTTCACTCTCACCATCAGCAGCCTAGAGCCTGAA |
| | | | GATTTTGCAGTTTATTACTGTATGCAGTGGAGTGGTTACCCATACACGTT |
| | | | CGGCGGAGGGACCAAGGTGGAGATCAAA |
| 126 | | H Chain AA | EVQLVESGGGLVQPGGSLRLSCAASGFQFS |
| | | | SFAMSWVRQAPGKGLEWVAKISPGGSYTYY |
| | | | SDTVTGRFTISRDNAKNSLYLQMNSLRAED |
| | | | TAVYYCARQLWGYYALDIWGQGTTVTVSS |
| 127 | | H Chain | ATGGAGTTTGGCCTGAGCTGGGTTTTCCTTGTTGCTATTTTAGAAGGTGT |
| | | Nucleotide | CCAGTGTGAGGTGCAGCTGGTGGAGTCTGGGGGAGGCTTGGTCCAGCCTG |
| | | | GGGGGTCCCTGAGACTCTCCTGTGCAGCCTCTGGATTCCAGTTTAGTAGC |
| | | | TTTGCCATGTCTTGGGTCCGCCAGGCTCCAGGGAAGGGGCTGGAGTGGGT |
| | | | GGCCAAAATTAGTCCCGGTGGGAGTTACACCTACTATAGCGACACTGTGA |
| | | | CGGGCCGATTCACCATCTCCAGAGACAACGCCAAGAACTCACTGTATCTG |
| | | | CAAATGAACAGCCTGAGAGCCGAGGACACGGCTGTGTATTACTGTGCGAG |
| | | | ACAGTTATGGGGGTACTATGCTCTTGACATTTGGGGCCAAGGGACCACGG |
| | | | TCACCGTCTCCTCA |
| 128 | AME-22a | L Chain AA | EIVLTQSPATLSLSPGERATLSCSASYSVS |
| | | | YMYWYQQKPGQAPRLLIYDFSNLASGIPAR |
| | | | FSGSGSGTDFTLTISSLEPEDFAVYYCMQW |
| | | | SGYPYTFGGGTKVEIK |
| 129 | | L Chain | ATGGAAGCCCCAGCGCAGCTTCTCTTCCTCCTGCTACTCTGGCTCCCAGA |
| | | Nucleotide | TACCACCGGAGAAATTGTGTTGACACAGTCTCCAGCCACCCTGTCTTTGT |
| | | | CTCCAGGGGAAAGAGCCACCCTCTCCTGCAGTGCCAGCTACAGTGTAAGT |
| | | | TACATGTACTGGTACCAACAGAAACCTGGCCAGGCTCCCAGGCTCCTCAT |
| | | | CTATGACTTCTCCAACCTGGCTTCTGGCATCCCAGCCAGGTTCAGTGGCA |
| | | | GTGGGTCTGGGACAGACTTCACTCTCACCATCAGCAGCCTAGAGCCTGAA |
| | | | GATTTTGCAGTTTATTACTGTATGCAGTGGAGTGGTTACCCATACACGTT |
| | | | CGGCGGAGGGACCAAGGTGGAGATCAAA |
| 130 | | H Chain AA | EVQLVESGGGLVQPGGSLRLSCAASGFQFS |
| | | | PFAMSWVRQAPGKGLEWVAKISPGGSWTYY |
| | | | PDTDTGRFTISRDNAKNSLYLQMNSLRAED |
| | | | TAVYYCARQLWGYYALDFWGQGTTVTVSS |
| 131 | | H Chain | ATGGAGTTTGGCCTGAGCTGGGTTTTCCTTGTTGCTATTTTAGAAGGTGT |
| | | Nucleotide | CCAGTGTGAGGTGCAGCTGGTGGAGTCTGGGGGAGGCTTGGTCCAGCCTG |
| | | | GGGGGTCCCTGAGACTCTCCTGTGCAGCCTCTGGATTCCAGTTTAGTCCC |
| | | | TTTGCCATGTCTTGGGTCCGCCAGGCTCCAGGGAAGGGGCTGGAGTGGGT |
| | | | GGCCAAAATTAGTCCCGGTGGGAGTTGGACCTACTATCCTGACACTGACA |
| | | | CGGGCCGATTCACCATCTCCAGAGACAACGCCAAGAACTCACTGTATCTG |
| | | | CAAATGAACAGCCTGAGAGCCGAGGACACGGCTGTGTATTACTGTGCGAG |
| | | | ACAGTTATGGGGGTACTATGCTCTTGACTTCTGGGGCCAAGGGACCACGG |
| | | | TCACCGTCTCCTCA |

TABLE 7
Amino acid sequence of a human light chain
framework region L6 with interspersed CDR sequences labeled

```
        (FRL1-SEQ ID NO: 105) CDRL1 (FRL2-SEQ ID NO: 106) CDRL2
EIVLTQSPATLSLSPGERATLSCXXXXXXXXXXWYQQKPGQAPRLLIYXXXXXXX
                (FRL3-SEQ ID NO: 107) CDRL3 (FRL4-SEQ ID NO: 108)
GIPARFSGSGSGTDFTLTISSLEPEDFAVYYCXXXXXXXXXFGGGTKVEIK
```

TABLE 8
Amino acid sequence of a human heavy chain
framework region VH3-7 with interspersed CDR sequences labeled

```
        (FRH1-SEQ ID NO: 109) CDRH1 (FRH2-SEQ ID NO: 110)
EVQLVESGGGLVQPGGSLRLSCAASXXXXXXXXXWVRQAPGKGLEWVA
          CDRH2 (FRH3-SEQ ID NO: 111)
XXXXXXXXXXXXXXXXXRFTISRDNAKNSLYLQMNSLRAEDTAVYYCAR
      CDRH3 (FRH4-SEQ ID NO: 112)
XXXXXXXXXWGQGTTVTVSS
```

TABLE 9
CDR Sequences

| SEQ | | |
|---|---|---|
| ID NO: | CDR | AA Sequence* |
| 132 | CDRL1 | SX₁X₂X₃X₄VX₅YMY |
| 133 | CDRL2 | DX₆SX₇LX₈S |
| 134 | CDRL3 | X₉X₁₀WSGYPYT |
| 135 | CDRH1 | GFX₁₁X₁₂SX₁₃FAX₁₄S |
| 136 | CDRH2 | KX₁₅SX₁₆GGSX₁₇X₁₈YX₁₉X₂₀DTX₂₁X₂₂X₂₃ |
| 137 | CDRH3 | QLWGX₂₄YALDX₂₅ |

*X denotes any suitable amino acid with exemplary, non-limiting amino acid substitutions shown in the sequences disclosed in SEQ ID NOS: 1-92 of Tables 1 and 2 and in Tables 3, 4, and 5A. In addition, X can have the following values:
X₁ = A or G
X₂ = S or R
X₃ = H, I, S, or Y
X₄ = S or Y
X₅ = S or F
X₆ = F, L, M, or T
X₇ = N or E
X₈ = A or T
X₉ = M, C, or S
X₁₀ = Q or C
X₁₁ = T or Q
X₁₂ = F, S, or T
X₁₃ = S or P
X₁₄ = L or M
X₁₅ = A or I
X₁₆ = S or P
X₁₇ = Y or W
X₁₈ = T, E, or Y
X₁₉ = Y or F
X₂₀ = P, S D, or Y
X₂₁ = V or D
X₂₂ = T or A
X₂₃ = G or P
X₂₄ = S, Y, T, or N
X₂₅ = Y, T, F, or I

AMINO ACID SEQUENCE OF IL-6 PROTEIN
SEQ ID NO: 115
MNSFSTSAFGPVAFSLGLLLVLPAAFPAPVPPGEDSKDVAAPH
RQPLTSSERIDKQIRYILDGISALRKETCNKSNMCESSKEALA
ENNLNLPKMAEKDGCFQSGFNEETCLVKIITGLLEFEVYLEYL
QNRFESSEEQARAVQMSTKVLIQFLQKKAKNLDAITTPDPTTN
ASLLTKLQAQNQWLQDMTTHLILRSFKEFLQSSLRALRQM

In accordance with the present invention, the anti-IL-6 cCLB-8 antibody comprises an antibody in which the variable region or CDRs are derived from the murine CLB-8 antibody capable of binding to and inhibiting the function of human IL-6 and the framework and constant regions of the antibody are derived from one or more human antibodies. The variable region or CDRs derived from the murine CLB-8 antibody preferably have from about 90% to about 100% identity with the variable region or CDRs of the murine CLB-8 antibody, although any and all modifications, including substitutions, insertions and deletions, are contemplated so long as the chimeric antibody maintains the ability to bind to and inhibit IL-6. The regions of the chimeric, humanized or CDR-grafted antibodies that are derived from human antibodies need not have 100% identity with the human antibodies. In a preferred embodiment, as many of the human amino acid residues as possible are retained in order than immunogenicity is negligible, but the human residues, in particular residues of the framework region, are substituted as required and as taught herein below in accordance with the present invention. Such modifications as disclosed herein are necessary to support the antigen binding site formed by the CDRs while simultaneously maximizing the humanization of the antibody.

The CLB-8 murine monoclonal antibody against human IL-6 is known in the art (Brakenhoff et al, J. Immunol 145:561 (1990)). The present invention discloses chimeric, humanized or CDR grafted antibodies derived from the CDR regions of the CLB-8 murine monoclonal antibody and methods for preparing such antibodies. Each of the heavy and light chain variable regions contain three CDRs that combine to form the antigen binding site. The three CDRs are surrounded by four framework (FR) regions that primarily function to support the CDRs. The sequences of the CDRs within the sequences of the variable regions of the heavy and light chains can be identified by computer-assisted alignment according to Kabat et al. (1987) in Sequences of Proteins of Immunological Interest, 4^{th} ed., United States Department of Health and Human Services, U.S. Government Printing Office, Washington, D.C., or by molecular modeling of the variable regions, for example utilizing the ENCAD program as described by Levitt (1983) J. Mol. Biol. 168:595.

In a preferred embodiment the CDRs are derived from murine monoclonal antibody CLB-8.

The preferred heavy chain CDRs have the following sequences:

CDR1
(SEQ ID NO: 141)
SFAMS
CDR2
(SEQ ID NO: 142)
EISSGGSYTYYPDTVTG
CDR3
(SEQ ID NO: 143)
GLWGYYALDY

The preferred light chain CDRs have the following sequences:

CDR1
(SEQ. ID NO: 144)
SASSSVSYMY
CDR2
(SEQ. ID NO: 145)
DTSNLAS
CDR3
(SEQ. ID NO: 146)
QQWSGYPYT

The sequences of the CDRs of the murine CLB-8 antibody, may be modified by insertions, substitutions and deletions to the extent that the CDR-grafted antibody maintains the ability to bind to and inhibit human IL-6. The ordinarily skilled artisan can ascertain the maintenance of this activity by performing the functional assays described herein below. The CDRs can have, for example, from about 50% to about 100% homology to the CDRs of SEQ ID NOS: 141-146. In a preferred embodiment the CDRs have from about 80% to about 100% homology to the CDRs of SEQ ID NOS: 141-146. In a more preferred embodiment the CDRs have from about 90% to about 100% homology to the CDRs of SEQ ID NOS: 141-146. In a most preferred embodiment the CDRs have from about 100% homology to the CDRs of SEQ ID NOS: 141-146.

Alternatively, the entire heavy chain variable region and light chain variable region of the murine CLB-8 antibody (SEQ ID NOS. 139 and 140) may be combined with the human constant and framework regions to form the chimeric cCLB-8 antibody of the present invention.

The preferred heavy chain variable region and light chain variable region of the murine CLB-8 antibody (SEQ. ID NOS. 139 and 140)

```
Heavy chain variable region (SEQ. ID NOS. 139):
  1 EVQLVESGGK LLKPGGSLKL SCAASGFTFS SFAMSWFRQS PEKRLEWVAE ISSGGSYTYY
61 PDTVTGRFTI SRDNAKNTLY LEMSSLRSED TAMYYCARGL WGYYALDYWG QGTSVTVSS
Light chain variable region (SEQ. ID NOS. 140):
  1 QIVLIQSPAI MSASPGEKVT MTCSASSSVS YMYWYQQKPG SSPRLLIYDT SNLASGVPVR
61 FSGSGSGTSY SLTISRMEAE DAATYYCQQW SGYPYTFGGG TKLEIK
```

An anti-IL-6 antibody according to the present invention includes any protein or peptide containing a molecule that comprises at least a portion of an immunoglobulin molecule, such as but not limited to, at least one ligand binding portion (LBP), such as but not limited to, a complementarity determining region (CDR) of a heavy or light chain or a ligand binding portion thereof, a heavy chain or light chain variable region, a framework region (e.g., FR1, FR2, FR3, FR4 or fragment thereof, or as shown in SEQ ID NOS: 105-112, further optionally comprising at least one substitution, insertion or deletion), a heavy chain or light chain constant region, (e.g., comprising at least one CH1, hinge1, hinge2, hinge3, hinge4, CH2, or CH3 or fragment thereof, further optionally comprising at least one substitution, insertion or deletion), or any portion thereof, that can be incorporated into an antibody of the present invention. An antibody of the invention can include or be derived from any mammal, such as but not limited to, a human, a mouse, a rabbit, a rat, a rodent, a primate, or any combination thereof, and the like.

The isolated antibodies of the present invention comprise the antibody amino acid sequences disclosed herein encoded by any suitable polynucleotide, or any isolated or prepared antibody. Preferably, the human antibody or antigen-binding fragment binds human IL-6 and, thereby, partially or substantially neutralizes at least one biological activity of the protein. An antibody, or specified portion or variant thereof, that partially or preferably substantially neutralizes at least one biological activity of at least one IL-6 protein or fragment can bind the protein or fragment and thereby inhibit activities mediated through the binding of IL-6 to the IL-6 receptor or through other IL-6-dependent or mediated mechanisms. As used herein, the term “neutralizing antibody” refers to an antibody that can inhibit an IL-6-dependent activity by about 20-120%, preferably by at least about 10, 20, 30, 40, 50, 55, 60, 65, 70, 75, 80, 85, 90, 91, 92, 93, 94, 95, 96, 97, 98, 99, 100% or more depending on the assay. The capacity of an anti-IL-6 antibody to inhibit an IL-6-dependent activity is preferably assessed by at least one suitable IL-6 protein or receptor assay, as described herein and/or as known in the art. A human antibody of the invention can be of any class (IgG, IgA, IgM, IgE, IgD, etc.) or isotype and can comprise a kappa or lambda light chain. In one embodiment, the human antibody comprises an IgG heavy chain or defined fragment, for example, at least one of isotypes, IgG1, IgG2, IgG3 or IgG4 (e.g., γ1, γ2, γ3, or γ4). Antibodies of this type can be prepared by employing a transgenic mouse or other transgenic non-human mammal comprising at least one human light chain (e.g., IgG, IgA, and IgM) transgenes as described herein and/or as known in the art. In another embodiment, the anti-human IL-6 human antibody comprises an IgG1 heavy chain and an IgG1 light chain.

Generally, the human antibody or antigen-binding fragment of the present invention will comprise an antigen-binding region that comprises at least one human complementarity determining region (CDR1, CDR2 and CDR3) or variant of at least one heavy chain variable region and at least one human complementarity determining region (CDR1, CDR2 and CDR3) or variant of at least one light chain variable region. The CDR sequences may be derived from human germline sequences or closely match the germline sequences. For example, the CDRs from a synthetic library derived from the original mouse CDRs can be used. These CDRs may be formed by incorporation of conservative substitutions from the original mouse sequence. As a non-limiting example, the antibody or antigen-binding portion or variant can comprise at least one of the heavy chain CDR3 having an amino acid sequence selected from the group consisting of SEQ ID NOS: 79, 81, 83, 85, 87, 89, and 91, and/or a light chain CDR3 having an amino acid sequence selected from the group consisting of SEQ ID NOS:29, 31, 33, and 35. In a particular embodiment, the antibody or antigen-binding fragment can have an antigen-binding region that comprises at least a portion of at least one heavy chain CDR (i.e., CDR1, CDR2 and/or CDR3) having the amino acid sequence of the corresponding CDRs 1, 2, and/or 3 (e.g., SEQ ID NOS:37, 49, and 79). In another particular embodiment, the antibody or antigen-binding portion or variant can have an antigen-binding region that comprises at least a portion of at least one light chain CDR (i.e., CDR1, CDR2 and/or CDR3) having the amino acid sequence of the corresponding CDRs 1, 2 and/or 3 (e.g., SEQ ID NOS:1, 17, and 29).

At least one antibody of the present invention can be expressed in a modified form, such as a fusion protein, and can include not only secretion signals, but also additional heterologous functional regions. For instance, a region of additional amino acids, particularly charged amino acids, can be added to the N-terminus of an antibody to improve stability and persistence in the host cell, during purification, or during subsequent handling and storage. Also, peptide moieties can be added to an antibody of the present invention to facilitate purification. Such regions can be removed prior to final preparation of an antibody or at least one fragment thereof. Such methods are described in many standard laboratory manuals, such as Sambrook, supra, Chapters 17.29-17.42 and 18.1-18.74; Ausubel, supra, Chapters 16, 17 and 18.

Illustrative of cell cultures useful for the production of the antibodies, specified portions or variants thereof, are mammalian cells. Mammalian cell systems often will be in the form of monolayers of cells although mammalian cell suspensions or bioreactors can also be used. A number of suitable host cell lines capable of expressing intact glycosylated proteins have been developed in the art, and include the COS-1 (e.g., ATCC CRL 1650), COS-7 (e.g., ATCC CRL-1651), HEK293, BHK21 (e.g., ATCC CRL-10), CHO (e.g., ATCC CRL 1610) and BSC-1 (e.g., ATCC CRL-26) cell lines, Cos-7 cells, CHO cells, hep G2 cells, P3X63Ag8.653, SP2/0-Ag14, 293 cells, HeLa cells and the like, which are readily available from, for example, American Type Culture Collection, Manassas, Va. (www.atcc.org). Preferred host cells include cells of lymphoid origin, such as myeloma and lymphoma cells. Particularly preferred host cells are P3X63Ag8.653 cells (ATCC Accession Number CRL-1580) and SP2/0-Ag14 cells (ATCC Accession Number CRL-1851). In a particularly preferred embodiment, the recombinant cell is a P3X63Ab8.653 or a SP2/0-Ag14 cell.

Expression vectors for these cells can include one or more of the following expression control sequences, such as, but not limited to, an origin of replication; a promoter (e.g., late or early SV40 promoters, the CMV promoter (U.S. Pat. Nos. 5,168,062; 5,385,839), an HSV tk promoter, a pgk (phosphoglycerate kinase) promoter, an EF-1 alpha promoter (U.S. Pat. No. 5,266,491), at least one human immunoglobulin promoter; an enhancer, and/or processing information sites, such as ribosome binding sites, RNA splice sites, polyadenylation sites (e.g., an SV40 large T Ag poly A addition site), and transcriptional terminator sequences. See, e.g., Ausubel et al., supra; Sambrook, et al., supra. Other cells useful for production of nucleic acids or proteins of the present invention are known and/or available, for instance, from the American Type Culture Collection Catalogue of Cell Lines and Hybridomas (www.atcc.org) or other known or commercial sources.

### Purification of an Antibody

An anti-IL-6 antibody can be recovered and purified from recombinant cell cultures by well-known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, NY, (1997-2001), e.g., Chapters 1, 4, 6, 8, 9, 10, each entirely incorporated herein by reference.

Antibodies of the present invention include naturally purified products, products of chemical synthetic procedures, and products produced by recombinant techniques from a eukaryotic host, including, for example, yeast, higher plant, insect and mammalian cells. Depending upon the host employed in a recombinant production procedure, the antibody of the present invention can be glycosylated or can be non-glycosylated, with glycosylated preferred. Such methods are described in many standard laboratory manuals, such as Sambrook, supra, Sections 17.37-17.42; Ausubel, supra, Chapters 10, 12, 13, 16, 18 and 20, Colligan, Protein Science, supra, Chapters 12-14, all entirely incorporated herein by reference.

### Amino Acid Codes

The amino acids that make up anti-IL-6 antibodies of the present invention are often abbreviated. The amino acid designations can be indicated by designating the amino acid by its single letter code, its three letter code, name, or three nucleotide codon(s) as is well understood in the art (see Alberts, B., et al., Molecular Biology of The Cell, Third Ed., Garland Publishing, Inc., New York, 1994)

An anti-IL-6 antibody of the present invention can include one or more amino acid substitutions, deletions or additions, either from natural mutations or human manipulation, as specified herein. Amino acids in an anti-IL-6 antibody of the present invention that are essential for function can be identified by methods known in the art, such as site-directed mutagenesis or alanine-scanning mutagenesis (e.g., Ausubel, supra, Chapters 8, 15; Cunningham and Wells, Science 244:1081-1085 (1989)). The latter procedure introduces single alanine mutations at every residue in the molecule. The resulting mutant molecules are then tested for biological activity, such as, but not limited to, at least one IL-6 neutralizing activity. Sites that are critical for antibody binding can also be identified by structural analysis, such as crystallization, nuclear magnetic resonance or photoaffinity labeling (Smith, et al., J. Mol. Biol. 224:899-904 (1992) and de Vos, et al., Science 255:306-312 (1992)).

Non-limiting variants that can enhance or maintain at least one of the listed activities include, but are not limited to, any of the above polypeptides, further comprising at least one mutation corresponding to at least one substitution in the residues varied among the disclosed variant amino acid sequences.

In another aspect, the invention relates to human antibodies and antigen-binding fragments, as described herein, which are modified by the covalent attachment of an organic moiety. Such modification can produce an antibody or antigen-binding fragment with improved pharmacokinetic properties (e.g., increased in vivo serum half-life). The organic moiety can be a linear or branched hydrophilic polymeric group, fatty acid group, or fatty acid ester group. In particular embodiments, the hydrophilic polymeric group can have a molecular weight of about 800 to about 120,000 Daltons and can be a polyalkane glycol (e.g., polyethylene glycol (PEG), polypropylene glycol (PPG)), carbohydrate polymer, amino acid polymer or polyvinyl pyrolidone, and the fatty acid or fatty acid ester group can comprise from about eight to about forty carbon atoms.

The modified antibodies and antigen-binding fragments of the invention can comprise one or more organic moieties that are covalently bonded, directly or indirectly, to the antibody. Each organic moiety that is bonded to an antibody or antigen-binding fragment of the invention can independently be a hydrophilic polymeric group, a fatty acid group or a fatty acid ester group. As used herein, the term “fatty acid” encompasses mono-carboxylic acids and di-carboxylic acids. A “hydrophilic polymeric group,” as the term is used herein, refers to an organic polymer that is more soluble in water than in octane. For example, polylysine is more soluble in water than in octane. Thus, an antibody modified by the covalent attachment of polylysine is encompassed by the invention. Hydrophilic polymers suitable for modifying antibodies of the invention can be linear or branched and include, for example, polyalkane glycols (e.g., PEG, monomethoxy-polyethylene glycol (mPEG), PPG and the like), carbohydrates (e.g., dextran, cellulose, oligosaccharides, polysaccharides and the like), polymers of hydrophilic amino acids (e.g., polylysine, polyarginine, polyaspartate and the like), polyalkane oxides (e.g., polyethylene oxide, polypropylene oxide and the like) and polyvinyl pyrolidone. Preferably, the hydrophilic polymer that modifies the antibody of the invention has a molecular weight of about 800 to about 150,000 Daltons as a separate molecular entity. For example, PEG5000 and PEG20,000, wherein the subscript is the average molecular weight of the polymer in Daltons, can be used. The hydrophilic polymeric group can be substituted with one to about six alkyl, fatty acid or fatty acid ester groups. Hydrophilic polymers that are substituted with a fatty acid or fatty acid ester group can be prepared by employing suitable methods. For example, a polymer comprising an amine group can be coupled to a carboxylate of the fatty acid or fatty acid ester, and an activated carboxylate (e.g., activated with N, N-carbonyl diimidazole) on a fatty acid or fatty acid ester can be coupled to a hydroxyl group on a polymer.

Fatty acids and fatty acid esters suitable for modifying antibodies of the invention can be saturated or can contain one or more units of unsaturation. Fatty acids that are suitable for modifying antibodies of the invention include, for example, n-dodecanoate (C12, laurate), n-tetradecanoate (C14, myristate), n-octadecanoate (C18, stearate), n-eicosanoate (C20, arachidate), n-docosanoate (C22, behenate), n-triacontanoate (C30), n-tetracontanoate (C40), cis-Δ9-octadecanoate (C18, oleate), all cis-Δ5,8,11,14-eicosatetraenoate (C20, arachidonate), octanedioic acid, tetradecanedioic acid, octadecanedioic acid, docosanedioic acid, and the like. Suitable fatty acid esters include mono-esters of dicarboxylic acids that comprise a linear or branched lower alkyl group. The lower alkyl group can comprise from one to about twelve, preferably, one to about six, carbon atoms.

The modified human antibodies and antigen-binding fragments can be prepared using suitable methods, such as by reaction with one or more modifying agents. A “modifying agent” as the term is used herein, refers to a suitable organic group (e.g., hydrophilic polymer, a fatty acid, a fatty acid ester) that comprises an activating group. An “activating group” is a chemical moiety or functional group that can, under appropriate conditions, react with a second chemical group thereby forming a covalent bond between the modifying agent and the second chemical group. For example, amine-reactive activating groups include electrophilic groups, such as tosylate, mesylate, halo (chloro, bromo, fluoro, iodo), N-hydroxysuccinimidyl esters (NHS), and the like. Activating groups that can react with thiols include, for example, maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., Bioconjugate Techniques, Academic Press: San Diego, Calif. (1996)). An activating group can be bonded directly to the organic group (e.g., hydrophilic polymer, fatty acid, fatty acid ester), or through a linker moiety, for example, a divalent C1-C12 group wherein one or more carbon atoms can be replaced by a heteroatom, such as oxygen, nitrogen or sulfur. Suitable linker moieties include, for example, tetraethylene glycol, —(CH2)3-, —NH—(CH2)6-NH—, —(CH2)2-NH— and —CH2-O—CH2-CH2-O—CH2-CH2O—CH—NH—. Modifying agents that comprise a linker moiety can be produced, for example, by reacting a mono-Boc-alkyldiamine (e.g., mono-Boc-ethylenediamine, mono-Boc-diaminohexane) with a fatty acid in the presence of 1-ethyl-3-(3-dimethylaminopropyl) carbodiimide (EDC) to form an amide bond between the free amine and the fatty acid carboxylate. The Boc protecting group can be removed from the product by treatment with trifluoroacetic acid (TFA) to expose a primary amine that can be coupled to another carboxylate, as described, or can be reacted with maleic anhydride and the resulting product cyclized to produce an activated maleimido derivative of the fatty acid. (See, for example, Thompson, et al., WO 92/16221, the entire teachings of which are incorporated herein by reference.)

The modified antibodies of the invention can be produced by reacting a human antibody or antigen-binding fragment with a modifying agent. For example, the organic moieties can be bonded to the antibody in a non-site specific manner by employing an amine-reactive modifying agent, for example, an NHS ester of PEG. Modified human antibodies or antigen-binding fragments can also be prepared by reducing disulfide bonds (e.g., intra-chain disulfide bonds) of an antibody or antigen-binding fragment. The reduced antibody or antigen-binding fragment can then be reacted with a thiol-reactive modifying agent to produce the modified antibody of the invention. Modified human antibodies and antigen-binding fragments comprising an organic moiety that is bonded to specific sites of an antibody of the present invention can be prepared using suitable methods, such as reverse proteolysis (Fisch et al., Bioconjugate Chem., 3:147-153 (1992); Werlen et al., Bioconjugate Chem., 5:411-417 (1994); Kumaran et al., Protein Sci. 6(10):2233-2241 (1997); Itoh et al., Bioorg. Chem., 24(1): 59-68 (1996); Capellas et al., Biotechnol. Bioeng., 56(4):456-463 (1997)), and the methods described in Hermanson, G. T., Bioconjugate Techniques, Academic Press: San Diego, Calif. (1996).

### Coronavirus

The Coronavirus family contains the genera Alphacoronavirus, Betacoronavirus, Gammacoronavirus, and Deltacoronavirus. All of these genera contain pathogenic viruses that can infect a wide variety of animals, including birds, cats, dogs, cows, bats, and humans. These viruses cause a range of diseases including enteric and respiratory diseases. The host range is primarily determined by the viral spike protein (S protein), which mediates entry of the virus into host cells. Coronaviruses that can infect humans are found both in the genus Alphacoronavirus and the genus Betacoronavirus. Known coronaviruses that cause respiratory disease in humans are members of the genus Betacoronavirus. These include SARS-CoV-1, SARS-CoV-2 and MERS.

SARS-CoV-1 and SARS-CoV-2 can cause severe respiratory disease in humans. The viral spike protein expressed by these viruses binds to angiotensin-converting enzyme 2 (ACE2). In some embodiments, the invention provides methods of treating a human suspected of having an infection with a Betacoronavirus causing SARS. In particular embodiments, the Betacoronavirus expresses a spike glycoprotein (S protein) that binds to ACE2, specifically human ACE2.

In a preferred embodiment of the invention, the infection is SARS-CoV-2.

### SARS-CoV-1

In some embodiments, the invention provides for methods of treating a human suspected of having an infection with SARS-CoV-1 (severe acute respiratory syndrome coronavirus 1). SARS-CoV-1 is a positive-sense single-stranded RNA virus of the Betacoronavirus genus that causes SARS. In some embodiments, the invention provides for methods of treating a human having been diagnosed with SARS. The full genome sequences of various isolates from infected human patients are available from GenBank. The reference genome has the NCBI Reference Sequence ID NC_004718.

### SARS-CoV-2

In some embodiments, the invention provides for methods of treating a human suspected of having an infection with SARS-CoV-2 (severe acute respiratory syndrome coronavirus 2). SARS-CoV-2 (synonyms: 2019-nCoV, HCoV-19) is a positive-sense single-stranded RNA virus of the Betacoronavirus genus that causes the coronavirus disease termed COVID-19. In some embodiments, the invention provides for methods of treating a human having been diagnosed with COVID-19. The full genome sequences of various isolates from infected human patients are available from GenBank. The reference genome has the GenBank ID MN908947 (NCBI Reference Sequence ID NC_045512).

Symptoms of infection with SARS-CoV-2 may include, but are not limited to fever, chest pains, dry cough, dyspnea, headache, and hypoxemia. A human may be infected with SARS-CoV-2 but, at least initially, not demonstrate any symptoms of COVID-19. The invention encompasses the treatment of these patients.

### Identifying Subjects in Need of Treatment

In certain embodiments, the methods of treatment described herein may include steps for identifying a human suspected of having an infection with a coronavirus causing severe respiratory disease. Such identification steps are typically highly specific for a particular virus. For example, they may include testing for the presence of specific viruses known to cause respiratory disease, including coronaviruses such as MERS, SARS-CoV-1 and SARS-CoV-2. In some embodiments, testing includes step for specifically identifying SARS-CoV-1 and SARS-CoV-2. In a particular embodiment, a test for use with the invention is specific for SARS-CoV-2.

Identification may include detecting symptoms of the virus infection, and detecting virus-specific antigens, antibodies or nucleic acids in a biological sample. The term “biological sample” and used herein may include cell culture or extracts thereof; biopsied material obtained from a human; and blood, mucus, saliva, urine, feces, semen, tears or other body fluids or extracts thereof. In particular embodiments, steps for identifying humans suspected of having an infection with a coronavirus causing SARS may include real-time reverse transcription polymerase chain reaction (rRT-PCR). rRT-PCR may include detection of gene sequence specific to SARS-CoV-2, for example, those found in RdRP, ORF lab E and/or N genes. In some embodiments, multiple (e.g., 2 or 3) specific sequences in the same gene (e.g., the RdRP or N gene) are detected.

### Therapeutic Methods

As used herein, the term “treating” and its variants (e.g., “treat”, “treatment” etc.) is understood as the management and care of a patient for the purpose of combatting the disease, condition or disorder, including amelioration of one or more symptoms of a disease, condition or disorder.

As used herein, the term “prevent” and its variants (e.g., “preventing”) refers to the ability of a drug, or a combination of drugs, to stave off the occurrence of a clinically undesirable disease, disorder, symptom, or condition for a clinically significant period of time.

The term “therapeutically effective amount” used herein refers to the amount of anti-IL6 agent needed to treat or ameliorate the virus infection defined herein. The term “prophylactically effective amount” used herein refers to the amount of anti-IL6 agent needed to prevent the virus infection described herein. The exact dosage will generally be dependent on the patient's status at the time of administration. Factors that may be taken into consideration when determining dosage include the severity of the virus state in the human, the general health of the human, the age, weight, gender, diet, time, frequency and route of administration, drug combinations, reaction sensitivities and the human's tolerance or response to therapy. The precise amount can be determined by routine experimentation, but may ultimately lie with the judgement of the clinician. Particularly useful doses for use in this invention are set out below. The anti-IL6 agent may be administered individually to a human or may be administered in combination with one or more further agents (see “Combinations” section below). Alternatively, the anti-IL6 agent may be administered as a monotherapy.

### Dosage Regimen, Pharmaceutical Form and Modes of Administration

Based on the preliminary findings reported, the baseline blood IL-6 level is significantly elevated to hundreds pg/mL magnitude in severe and critically ill COVID-19 patients, which is much higher than the baseline IL-6 levels seen in subjects having rheumatoid arthritis (RA). In addition, IL-6 levels in bronchoalveolar lavage fluid in patients at risk for ARDS and with established ARDS could raise to thousands pg/mL and remain above the normal range for up to 3 weeks in patients with persistent ARDS.

Anti-IL-6 antibody compounds, compositions or combinations of the present invention can further comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of, and methods of preparing such sterile solutions are well known in the art, such as, but limited to, Gennaro, Ed., Remington's Pharmaceutical Sciences, 18th Edition, Mack Publishing Co. (Easton, Pa.) 1990. Pharmaceutically acceptable carriers can be routinely selected that are suitable for the mode of administration, solubility and/or stability of the anti-IL-6 antibody, fragment or variant composition as well known in the art or as described herein.

Pharmaceutical excipients and additives useful in the present composition include, but are not limited to, proteins, peptides, amino acids, lipids, and carbohydrates (e.g., sugars, including monosaccharides, di-, tri-, tetra-, and oligosaccharides; derivatized sugars, such as alditols, aldonic acids, esterified sugars and the like; and polysaccharides or sugar polymers), which can be present singly or in combination, comprising alone or in combination 1-99.99% by weight or volume. Exemplary protein excipients include serum albumin, such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

Carbohydrate excipients suitable for use in the composition of the invention include, for example, monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. Preferred carbohydrate excipients for use in the present invention are mannitol, trehalose, and raffinose.

Anti-IL-6 antibody compositions can also include a buffer or a pH adjusting agent; typically, the buffer is a salt prepared from an organic acid or base. Representative buffers include organic acid salts, such as salts of citric acid, ascorbic acid, gluconic acid, carbonic acid, tartaric acid, succinic acid, acetic acid, or phthalic acid; Tris, tromethamine hydrochloride, or phosphate buffers. Preferred buffers for use in the present compositions are organic acid salts, such as citrate.

Additionally, anti-IL-6 antibody compositions of the invention can include polymeric excipients/additives, such as polyvinylpyrrolidones, ficolls (a polymeric sugar), dextrates (e.g., cyclodextrins, such as 2-hydroxypropyl-β-cyclodextrin), polyethylene glycols, flavoring agents, antimicrobial agents, sweeteners, antioxidants, antistatic agents, surfactants (e.g., polysorbates, such as “TWEEN 20” and “TWEEN 80”), lipids (e.g., phospholipids, fatty acids), steroids (e.g., cholesterol), and chelating agents (e.g., EDTA).

These and additional known pharmaceutical excipients and/or additives suitable for use in the anti-IL-6 antibody, portion or variant compositions according to the invention are known in the art, e.g., as listed in “Remington: The Science & Practice of Pharmacy”, 19th ed., Williams & Williams, (1995), and in the “Physician's Desk Reference”, 52nd ed., Medical Economics, Montvale, N.J. (1998), the disclosures of which are entirely incorporated herein by reference. Preferred carrier or excipient materials are carbohydrates (e.g., saccharides and alditols) and buffers (e.g., citrate) or polymeric agents. An exemplary carrier molecule is the mucopolysaccharide, hyaluronic acid, which may be useful for intraarticular delivery.

### Formulations

As noted above, the invention provides for stable formulations, which preferably comprise a phosphate buffer with saline or a chosen salt, as well as preserved solutions and formulations containing a preservative as well as multi-use preserved formulations suitable for pharmaceutical or veterinary use, comprising at least one anti-IL-6 antibody in a pharmaceutically acceptable formulation. Preserved formulations contain at least one known preservative or optionally selected from the group consisting of at least one phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate and thimerosal, polymers, or mixtures thereof in an aqueous diluent. Any suitable concentration or mixture can be used as known in the art, such as about 0.0015%, or any range, value, or fraction therein. Non-limiting examples include, no preservative, about 0.1-2% m-cresol (e.g., 0.2, 0.3. 0.4, 0.5, 0.9, 1.0%), about 0.1-3% benzyl alcohol (e.g., 0.5, 0.9, 1.1, 1.5, 1.9, 2.0, 2.5%), about 0.001-0.5% thimerosal (e.g., 0.005, 0.01), about 0.001-2.0% phenol (e.g., 0.05, 0.25, 0.28, 0.5, 0.9, 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, 1.0%), and the like.

As noted above, the invention provides an article of manufacture, comprising packaging material and at least one vial comprising a solution of at least one anti-IL-6 antibody with the prescribed buffers and/or preservatives, optionally in an aqueous diluent, wherein said packaging material comprises a label that indicates that such solution can be held over a period of 1, 2, 3, 4, 5, 6, 9, 12, 18, 20, 24, 30, 36, 40, 48, 54, 60, 66, 72 hours or greater. The invention further comprises an article of manufacture, comprising packaging material, a first vial comprising lyophilized at least one anti-IL-6 antibody, and a second vial comprising an aqueous diluent of prescribed buffer or preservative, wherein said packaging material comprises a label that instructs a patient to reconstitute the at least one anti-IL-6 antibody in the aqueous diluent to form a solution that can be held over a period of twenty-four hours or greater.

The at least one anti-IL-6 antibody used in accordance with the present invention can be produced by recombinant means, including from mammalian cell or transgenic preparations, or can be purified from other biological sources, as described herein or as known in the art.

The range of at least one anti-IL-6 antibody in the use and method of the present invention includes amounts yielding upon reconstitution, if in a wet/dry system, concentrations from about 1.0 μg/ml to about 1000 mg/ml, although lower and higher concentrations are operable and are dependent on the intended delivery vehicle, e.g., solution formulations will differ from transdermal patch, pulmonary, transmucosal, or osmotic or micro pump methods.

Preferably, the aqueous diluent optionally further comprises a pharmaceutically acceptable preservative. Preferred preservatives include those selected from the group consisting of phenol, m-cresol, p-cresol, o-cresol, chlorocresol, benzyl alcohol, alkylparaben (methyl, ethyl, propyl, butyl and the like), benzalkonium chloride, benzethonium chloride, sodium dehydroacetate and thimerosal, or mixtures thereof. The concentration of preservative used in the formulation is a concentration sufficient to yield an anti-microbial effect. Such concentrations are dependent on the preservative selected and are readily determined by the skilled artisan.

Other excipients, e.g., isotonicity agents, buffers, antioxidants, and preservative enhancers, can be optionally and preferably added to the diluent. An isotonicity agent, such as glycerin, is commonly used at known concentrations. A physiologically tolerated buffer is preferably added to provide improved pH control. The formulations can cover a wide range of pHs, such as from about pH 4 to about pH 10, and preferred ranges from about pH 5 to about pH 9, and a most preferred range of about 6.0 to about 8.0. Preferably, the formulations of the present invention have a pH between about 6.8 and about 7.8. Preferred buffers include phosphate buffers, most preferably, sodium phosphate, particularly, phosphate buffered saline (PBS).

Other additives, such as a pharmaceutically acceptable solubilizers like Tween 20 (polyoxyethylene (20) sorbitan monolaurate), Tween 40 (polyoxyethylene (20) sorbitan monopalmitate), Tween 80 (polyoxyethylene (20) sorbitan monooleate), Pluronic F68 (polyoxyethylene polyoxypropylene block copolymers), and PEG (polyethylene glycol) or non-ionic surfactants, such as polysorbate 20 or 80 or poloxamer 184 or 188, Pluronic® polyls, other block co-polymers, and chelators, such as EDTA and EGTA, can optionally be added to the formulations or compositions to reduce aggregation. These additives are particularly useful if a pump or plastic container is used to administer the formulation. The presence of pharmaceutically acceptable surfactant mitigates the propensity for the protein to aggregate.

The formulations of the present invention can be prepared by a process which comprises mixing at least one anti-IL-6 antibody and a preservative selected from the group consisting of phenol, m-cresol, p-cresol, o-cresol, chlorocresol, benzyl alcohol, alkylparaben, (methyl, ethyl, propyl, butyl and the like), benzalkonium chloride, benzethonium chloride, sodium dehydroacetate and thimerosal or mixtures thereof in an aqueous diluent. Mixing the at least one anti-IL-6 antibody and preservative in an aqueous diluent is carried out using conventional dissolution and mixing procedures. To prepare a suitable formulation, for example, a measured amount of at least one anti-IL-6 antibody in buffered solution is combined with the desired preservative in a buffered solution in quantities sufficient to provide the protein and preservative at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The formulations according to the invention can be provided to patients as clear solutions or as dual vials comprising a vial of lyophilized at least one anti-IL-6 antibody that is reconstituted with a second vial containing water, a preservative and/or excipients, preferably, a phosphate buffer and/or saline and a chosen salt, in an aqueous diluent. Either a single solution vial or dual vial requiring reconstitution can be reused multiple times and can suffice for a single or multiple cycles of patient treatment and thus can provide a more convenient treatment regimen than currently available.

The present claimed products are useful for administration over a period ranging from immediate to twenty-four hours or greater. Accordingly, the presently claimed articles of manufacture offer significant advantages to the patient. Formulations of the invention can optionally be safely stored at temperatures of from about 2° C. to about 40° C. and retain the biological activity of the antibody for extended periods of time, thus allowing a package label indicating that the solution can be held and/or used over a period of 6, 12, 18, 24, 36, 48, 72, or 96 hours or greater. If preserved diluent is used, such label can include use up to 1-12 months, one-half, one and a half, and/or two years.

The solutions of at least one anti-IL-6 antibody of the invention can be prepared by a process that comprises mixing at least one antibody in an aqueous diluent. Mixing is carried out using conventional dissolution and mixing procedures. To prepare a suitable diluent, for example, a measured amount of at least one antibody in water or buffer is combined in quantities sufficient to provide the protein and, optionally, a preservative or buffer at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The antibody can be provided to patients as clear solutions or as dual vials comprising a vial of lyophilized anti-IL-6 antibody that is reconstituted with a second vial containing the aqueous diluent. Either a single solution vial or dual vial requiring reconstitution can be reused multiple times and can suffice for a single or multiple cycles of patient treatment and thus provides a more convenient treatment regimen than currently available.

The antibody can be provided indirectly to patients by providing to pharmacies, clinics, or other such institutions and facilities, clear solutions or dual vials comprising a vial of lyophilized anti-IL-6 antibody that is reconstituted with a second vial containing the aqueous diluent. The clear solution in this case can be up to one liter or even larger in size, providing a large reservoir from which smaller portions of the antibody solution can be retrieved one or multiple times for transfer into smaller vials and provided by the pharmacy or clinic to their customers and/or patients.

Recognized devices comprising single vial systems include pen-injector devices for delivery of a solution, such as BD Pens, BD Autojector®, Humaject®, NovoPen®, B-D®Pen, AutoPen®, and OptiPen®, GenotropinPen®, Genotronorm Pen®, Humatro Pen®, Reco-Pen®, Roferon Pen®, Biojector®, Iject®, J-tip Needle-Free Injector®, Intraject®, Medi-Ject®, e.g., as made or developed by Becton Dickensen (Franklin Lakes, N.J., www.bectondickenson.com), Disetronic (Burgdorf, Switzerland, www.disetronic.com; Bioject, Portland, Oreg. (www.bioject.com); National Medical Products, Weston Medical (Peterborough, UK, www.weston-medical.com), Medi-Ject Corp (Minneapolis, Minn., www.mediject.com), and similarly suitable devices, such as OnePress™. Recognized devices comprising a dual vial system include those pen-injector systems for reconstituting a lyophilized drug in a cartridge for delivery of the reconstituted solution, such as the HumatroPen®. Examples of other devices suitable include pre-filled syringes, auto-injectors, needle free injectors and needle free IV infusion sets.

The products used in the presently claimed uses and methods include packaging material. The packaging material provides, in addition to the information required by the regulatory agencies, the conditions under which the product can be used. The packaging material provides instructions to the patient to reconstitute the anti-IL-6 antibody in the aqueous diluent to form a solution and to use the solution over a period of 2-24 hours or greater for the two vial, wet/dry, product. For the single vial, solution product, the label indicates that such solution can be used over a period of 2-24 hours or greater. The products are useful for human pharmaceutical product use.

The formulations used in the present invention can be prepared by a process that comprises mixing an anti-IL-6 antibody and a selected buffer, preferably, a phosphate buffer containing saline or a chosen salt. Mixing the anti-IL-6 antibody and buffer in an aqueous diluent is carried out using conventional dissolution and mixing procedures. To prepare a suitable formulation, for example, a measured amount of at least one antibody in water or buffer is combined with the desired buffering agent in water in quantities sufficient to provide the protein and buffer at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The claimed stable or preserved formulations can be provided to patients as clear solutions or as dual vials comprising a vial of lyophilized an anti-IL-6 antibody that is reconstituted with a second vial containing a preservative or buffer and excipients in an aqueous diluent. Either a single solution vial or dual vial requiring reconstitution can be reused multiple times and can suffice for a single or multiple cycles of patient treatment and thus provides a more convenient treatment regimen than currently available.

Other formulations or methods of stabilizing the anti-IL-6 antibody may result in other than a clear solution of lyophilized powder comprising the antibody. Among non-clear solutions are formulations comprising particulate suspensions, said particulates being a composition containing the anti-IL-6 antibody in a structure of variable dimension and known variously as a microsphere, microparticle, nanoparticle, nanosphere, or liposome. Such relatively homogenous, essentially spherical, particulate formulations containing an active agent can be formed by contacting an aqueous phase containing the active agent and a polymer and a nonaqueous phase followed by evaporation of the nonaqueous phase to cause the coalescence of particles from the aqueous phase as taught in U.S. Pat. No. 4,589,330. Porous microparticles can be prepared using a first phase containing active agent and a polymer dispersed in a continuous solvent and removing said solvent from the suspension by freeze-drying or dilution-extraction-precipitation as taught in U.S. Pat. No. 4,818,542. Preferred polymers for such preparations are natural or synthetic copolymers or polymers selected from the group consisting of gelatin agar, starch, arabinogalactan, albumin, collagen, polyglycolic acid, polylactic aced, glycolide-L(-) lactide poly(episilon-caprolactone, poly(epsilon-caprolactone-CO-lactic acid), poly(epsilon-caprolactone-CO-glycolic acid), poly(B-hydroxy butyric acid), polyethylene oxide, polyethylene, poly(alkyl-2-cyanoacrylate), poly(hydroxyethyl methacrylate), polyamides, poly(amino acids), poly(2-hydroxyethyl DL-aspartamide), poly(ester urea), poly(L-phenylalanine/ethylene glycol/1,6-diisocyanatohexane) and poly(methyl methacrylate). Particularly preferred polymers are polyesters, such as polyglycolic acid, polylactic aced, glycolide-L(-) lactide poly(episilon-caprolactone, poly(epsilon-caprolactone-CO-lactic acid), and poly(epsilon-caprolactone-CO-glycolic acid. Solvents useful for dissolving the polymer and/or the active include: water, hexafluoroisopropanol, methylenechloride, tetrahydrofuran, hexane, benzene, or hexafluoroacetone sesquihydrate. The process of dispersing the active containing phase with a second phase may include pressure forcing said first phase through an orifice in a nozzle to affect droplet formation.

Dry powder formulations may result from processes other than lyophilization, such as by spray drying or solvent extraction by evaporation or by precipitation of a crystalline composition followed by one or more steps to remove aqueous or nonaqueous solvent. Preparation of a spray-dried antibody preparation is taught in U.S. Pat. No. 6,019,968. The antibody-based dry powder compositions may be produced by spray drying solutions or slurries of the antibody and, optionally, excipients, in a solvent under conditions to provide a respirable dry powder. Solvents may include polar compounds, such as water and ethanol, which may be readily dried. Antibody stability may be enhanced by performing the spray drying procedures in the absence of oxygen, such as under a nitrogen blanket or by using nitrogen as the drying gas. Another relatively dry formulation is a dispersion of a plurality of perforated microstructures dispersed in a suspension medium that typically comprises a hydrofluoroalkane propellant as taught in WO 9916419. The stabilized dispersions may be administered to the lung of a patient using a metered dose inhaler. Equipment useful in the commercial manufacture of spray dried medicaments are manufactured by Buchi Ltd. or Niro Corp.

An anti-IL-6 antibody in either the stable or preserved formulations or solutions described herein, can be administered to a patient in accordance with the present invention via a variety of delivery methods including SC or IM injection; transdermal, pulmonary, transmucosal, implant, osmotic pump, cartridge, micro pump, or other means appreciated by the skilled artisan, as well-known in the art.

### Alternative Administration

Many known and developed modes can be used according to the present invention for administering pharmaceutically effective amounts of an anti-IL-6 antibody according to the present invention. While pulmonary administration is used in the following description, other modes of administration can be used according to the present invention with suitable results. IL-6 antibodies of the present invention can be delivered in a carrier, as a solution, emulsion, colloid, or suspension, or as a dry powder, using any of a variety of devices and methods suitable for administration by inhalation or other modes described here within or known in the art.

### Parenteral Formulations and Administration

Formulations for parenteral administration can contain as common excipients sterile water or saline, polyalkylene glycols, such as polyethylene glycol, oils of vegetable origin, hydrogenated naphthalenes and the like. Aqueous or oily suspensions for injection can be prepared by using an appropriate emulsifier or humidifier and a suspending agent, according to known methods. Agents for injection can be a non-toxic, non-orally administrable diluting agent, such as aqueous solution, a sterile injectable solution or suspension in a solvent. As the usable vehicle or solvent, water, Ringer's solution, isotonic saline, etc. are allowed; as an ordinary solvent or suspending solvent, sterile involatile oil can be used. For these purposes, any kind of involatile oil and fatty acid can be used, including natural or synthetic or semisynthetic fatty oils or fatty acids; natural or synthetic or semisynthetic mono- or di- or tri-glycerides. Parental administration is known in the art and includes, but is not limited to, conventional means of injections, a gas pressured needle-less injection device as described in U.S. Pat. No. 5,851,198, and a laser perforator device as described in U.S. Pat. No. 5,839,446 entirely incorporated herein by reference.

### Alternative Delivery

The invention further relates to the administration of an anti-IL-6 antibody by parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal means. An anti-IL-6 antibody composition can be prepared for use for parenteral (subcutaneous, intramuscular or intravenous) or any other administration particularly in the form of liquid solutions or suspensions; for use in vaginal or rectal administration particularly in semisolid forms, such as, but not limited to, creams and suppositories; for buccal, or sublingual administration, such as, but not limited to, in the form of tablets or capsules; or intranasally, such as, but not limited to, the form of powders, nasal drops or aerosols or certain agents; or transdermally, such as not limited to a gel, ointment, lotion, suspension or patch delivery system with chemical enhancers such as dimethyl sulfoxide to either modify the skin structure or to increase the drug concentration in the transdermal patch (Junginger, et al. In “Drug Permeation Enhancement;” Hsieh, D. S., Eds., pp. 59-90 (Marcel Dekker, Inc. New York 1994, entirely incorporated herein by reference), or with oxidizing agents that enable the application of formulations containing proteins and peptides onto the skin (WO 98/53847), or applications of electric fields to create transient transport pathways, such as electroporation, or to increase the mobility of charged drugs through the skin, such as iontophoresis, or application of ultrasound, such as sonophoresis (U.S. Pat. Nos. 4,309,989 and 4,767,402) (the above publications and patents being entirely incorporated herein by reference).

### Pulmonary/Nasal Administration

For pulmonary administration, preferably, an anti-IL-6 antibody composition is delivered in a particle size effective for reaching the lower airways of the lung or sinuses. According to the invention, an anti-IL-6 antibody can be delivered by any of a variety of inhalation or nasal devices known in the art for administration of a therapeutic agent by inhalation. These devices capable of depositing aerosolized formulations in the sinus cavity or alveoli of a patient include metered dose inhalers, nebulizers, dry powder generators, sprayers, and the like. Other devices suitable for directing the pulmonary or nasal administration of antibodies are also known in the art. All such devices can use formulations suitable for the administration for the dispensing of antibody in an aerosol. Such aerosols can be comprised of either solutions (both aqueous and non-aqueous) or solid particles.

Metered dose inhalers like the Ventolin® metered dose inhaler, typically use a propellent gas and require actuation during inspiration (See, e.g., WO 94/16970, WO 98/35888). Dry powder inhalers like Turbuhaler™ (Astra), Rotahaler® (Glaxo), Diskus® (Glaxo), Spiros™ inhaler (Dura), devices marketed by Inhale Therapeutics, and the Spinhaler® powder inhaler (Fisons), use breath-actuation of a mixed powder (U.S. Pat. No. 4,668,218 Astra, EP 237507 Astra, WO 97/25086 Glaxo, WO 94/08552 Dura, U.S. Pat. No. 5,458,135 Inhale, WO 94/06498 Fisons, entirely incorporated herein by reference). Nebulizers like AERx™ Aradigm, the Ultravent® nebulizer (Mallinckrodt), and the Acorn II® nebulizer (Marquest Medical Products) (U.S. Pat. No. 5,404,871 Aradigm, WO 97/22376), the above references entirely incorporated herein by reference, produce aerosols from solutions, while metered dose inhalers, dry powder inhalers, etc. generate small particle aerosols. These specific examples of commercially available inhalation devices are intended to be a representative of specific devices suitable for the practice of this invention, and are not intended as limiting the scope of the invention.

Preferably, a composition comprising an anti-IL-6 antibody is delivered by a dry powder inhaler or a sprayer. There are several desirable features of an inhalation device for administering an antibody of the present invention. For example, delivery by the inhalation device is advantageously reliable, reproducible, and accurate. The inhalation device can optionally deliver small dry particles, e.g., less than about 10 μm, preferably about 1-5 μm, for good respirability.

### Administration of IL-6 Antibody Compositions as a Spray

A spray including IL-6 antibody composition can be produced by forcing a suspension or solution of an anti-IL-6 antibody through a nozzle under pressure. The nozzle size and configuration, the applied pressure, and the liquid feed rate can be chosen to achieve the desired output and particle size. An electrospray can be produced, for example, by an electric field in connection with a capillary or nozzle feed. Advantageously, particles of an anti-IL-6 antibody composition delivered by a sprayer have a particle size less than about 10 μm, preferably, in the range of about 1 μm to about 5 μm, and, most preferably, about 2 μm to about 3 μm.

Formulations of an anti-IL-6 antibody composition suitable for use with a sprayer typically include antibody composition in an aqueous solution at a concentration of about 0.1 mg to about 100 mg of an anti-IL-6 antibody composition per ml of solution or mg/gm, or any range, value, or fraction therein. The formulation can include agents, such as an excipient, a buffer, an isotonicity agent, a preservative, a surfactant, and, preferably, zinc. The formulation can also include an excipient or agent for stabilization of the antibody composition, such as a buffer, a reducing agent, a bulk protein, or a carbohydrate. Bulk proteins useful in formulating antibody compositions include albumin, protamine, or the like. Typical carbohydrates useful in formulating antibody compositions include sucrose, mannitol, lactose, trehalose, glucose, or the like. The antibody composition formulation can also include a surfactant, which can reduce or prevent surface-induced aggregation of the antibody composition caused by atomization of the solution in forming an aerosol. Various conventional surfactants can be employed, such as polyoxyethylene fatty acid esters and alcohols, and polyoxyethylene sorbitol fatty acid esters. Amounts will generally range between 0.001 and 14% by weight of the formulation. Especially preferred surfactants for purposes of this invention are polyoxyethylene sorbitan monooleate, polysorbate 80, polysorbate 20, or the like. Additional agents known in the art for formulation of a protein, such as IL-6 antibodies, or specified portions or variants, can also be included in the formulation.

### Oral Formulations and Administration

Formulations for oral administration rely on the co-administration of adjuvants (e.g., resorcinols and nonionic surfactants, such as polyoxyethylene oleyl ether and n-hexadecylpolyethylene ether) to increase artificially the permeability of the intestinal walls, as well as the co-administration of enzymatic inhibitors (e.g., pancreatic trypsin inhibitors, diisopropylfluorophosphate (DFF) and trasylol) to inhibit enzymatic degradation. Formulations for delivery of hydrophilic agents including proteins and antibodies and a combination of at least two surfactants intended for oral, buccal, mucosal, nasal, pulmonary, vaginal transmembrane, or rectal administration are taught in U.S. Pat. No. 6,309,663. The active constituent compound of the solid-type dosage form for oral administration can be mixed with at least one additive, including sucrose, lactose, cellulose, mannitol, trehalose, raffinose, maltitol, dextran, starches, agar, arginates, chitins, chitosans, pectins, gum tragacanth, gum arabic, gelatin, collagen, casein, albumin, synthetic or semisynthetic polymer, and glyceride. These dosage forms can also contain other type(s) of additives, e.g., inactive diluting agent, lubricant, such as magnesium stearate, paraben, preserving agent, such as sorbic acid, ascorbic acid, .alpha.-tocopherol, antioxidant such as cysteine, disintegrator, binder, thickener, buffering agent, sweetening agent, flavoring agent, perfuming agent, etc.

Tablets and pills can be further processed into enteric-coated preparations. The liquid preparations for oral administration include emulsion, syrup, elixir, suspension and solution preparations allowable for medical use. These preparations can contain inactive diluting agents ordinarily used in said field, e.g., water. Liposomes have also been described as drug delivery systems for insulin and heparin (U.S. Pat. No. 4,239,754). More recently, microspheres of artificial polymers of mixed amino acids (proteinoids) have been used to deliver pharmaceuticals (U.S. Pat. No. 4,925,673). Furthermore, carrier compounds described in U.S. Pat. No. 5,879,681 and U.S. Pat. No. 5,5,871,753 and used to deliver biologically active agents orally are known in the art.

Mucosal Formulations and Administration

A formulation for orally administering a bioactive agent encapsulated in one or more biocompatible polymer or copolymer excipients, preferably, a biodegradable polymer or copolymer, affording microcapsules which due to the proper size of the resultant microcapsules results in the agent reaching and being taken up by the folliculi lymphatic aggregati, otherwise known as the “Peyer's patch,” or “GALT” of the animal without loss of effectiveness due to the agent having passed through the gastrointestinal tract. Similar folliculi lymphatic aggregati can be found in the bronchei tubes (BALT) and the large intestine. The above-described tissues are referred to in general as mucosally associated lymphoreticular tissues (MALT). For absorption through mucosal surfaces, compositions and methods of administering at least one anti-IL-6 antibody include an emulsion comprising a plurality of submicron particles, a mucoadhesive macromolecule, a bioactive peptide, and an aqueous continuous phase, which promotes absorption through mucosal surfaces by achieving mucoadhesion of the emulsion particles (U.S. Pat. No. 5,514,670). Mucous surfaces suitable for application of the emulsions of the present invention can include corneal, conjunctival, buccal, sublingual, nasal, vaginal, pulmonary, stomachic, intestinal, and rectal routes of administration. Formulations for vaginal or rectal administration, e.g., suppositories, can contain as excipients, for example, polyalkyleneglycols, vaseline, cocoa butter, and the like. Formulations for intranasal administration can be solid and contain as excipients, for example, lactose or can be aqueous or oily solutions of nasal drops. For buccal administration, excipients include sugars, calcium stearate, magnesium stearate, pregelinatined starch, and the like (U.S. Pat. No. 5,849,695).

### Transdermal Formulations and Administration

For transdermal administration, the anti-IL-6 antibody is encapsulated in a delivery device, such as a liposome or polymeric nanoparticles, microparticle, microcapsule, or microspheres (referred to collectively as microparticles unless otherwise stated). A number of suitable devices are known, including microparticles made of synthetic polymers, such as polyhydroxy acids, such as polylactic acid, polyglycolic acid and copolymers thereof, polyorthoesters, polyanhydrides, and polyphosphazenes, and natural polymers, such as collagen, polyamino acids, albumin and other proteins, alginate and other polysaccharides, and combinations thereof (U.S. Pat. No. 5,814,599).

### Prolonged Administration and Formulations

It can be desirable to deliver the compounds of the present invention to the subject over prolonged periods of time, for example, for periods of one week to one year from a single administration. Various slow release, depot or implant dosage forms can be utilized. For example, a dosage form can contain a pharmaceutically acceptable non-toxic salt of the compounds that has a low degree of solubility in body fluids, for example, (a) an acid addition salt with a polybasic acid, such as phosphoric acid, sulfuric acid, citric acid, tartaric acid, tannic acid, pamoic acid, alginic acid, polyglutamic acid, naphthalene mono- or di-sulfonic acids, polygalacturonic acid, and the like; (b) a salt with a polyvalent metal cation, such as zinc, calcium, bismuth, barium, magnesium, aluminum, copper, cobalt, nickel, cadmium and the like, or with an organic cation formed from e.g., N,N′-dibenzyl-ethylenediamine or ethylenediamine; or (c) combinations of (a) and (b), e.g., a zinc tannate salt. Additionally, the compounds of the present invention or, preferably, a relatively insoluble salt, such as those just described, can be formulated in a gel, for example, an aluminum monostearate gel with, e.g., sesame oil, suitable for injection. Particularly preferred salts are zinc salts, zinc tannate salts, pamoate salts, and the like. Another type of slow release depot formulation for injection would contain the compound or salt dispersed for encapsulation in a slow degrading, non-toxic, non-antigenic polymer, such as a polylactic acid/polyglycolic acid polymer for example as described in U.S. Pat. No. 3,773,919. The compounds or, preferably, relatively insoluble salts, such as those described above, can also be formulated in cholesterol matrix silastic pellets, particularly for use in animals. Additional slow release, depot or implant formulations, e.g., gas or liquid liposomes, are known in the literature (U.S. Pat. No. 5,770,222 and “Sustained and Controlled Release Drug Delivery Systems”, J. R. Robinson ed., Marcel Dekker, Inc., N.Y., 1978).

### Combinations

In one embodiment of the invention, the anti-IL6 antibody or agent is co-administered with one or more further agents. The further agent may be an antiviral (including antiretroviral) agent. In an embodiment, the antiviral agent is a small molecule. The small molecule may be independently selected from the group consisting of itraconozole, favipiravir (e.g., Avigan), remdesivir, ifenprodil, chloroquine, umifenovir (e.g., Arbidol), APN01, galidesivir, ritonavir, BPI-002, OYA1, and SNG001. In an embodiment, the small molecule is favipiravir (e.g., Avigan). In an embodiment, the small molecule is remdesivir. In an embodiment, the small molecule is ifenprodil. In an embodiment, the small molecule is chloroquine. In an embodiment, the small molecule is umifenovir (e.g., Arbidol). In an embodiment, the small molecule is APN01. In an embodiment, the small molecule is galidesivir. In an embodiment, the small molecule is ritonavir. In an embodiment, the small molecule is BPI-002. In an embodiment, the small molecule is OYA1. In an embodiment, the small molecule is SNG001.

In an embodiment, the antiviral agent is a vaccine. The vaccine may be independently selected from the group consisting of a MERS-CoV vaccine, an Infectious Bronchitis Virus (IBV) vaccine, an RNA vaccine, e.g. an mRNA vaccine such as mRNA-1273, a DNA vaccine such as INO-4700 (GLS-5300) or INO-4800, a subunit vaccine, a live vaccine such as TNX-1800 and a recombinant vaccine. In one embodiment, the vaccine is co-administered with anti-IL6 agent for the prevention of infection of a human with SARS-CoV-1 and/or SARS-CoV-2.

In an embodiment, the antiviral agent is a protein. The protein may be a human recombinant protein such as AT-100 (rhSP-D). In another embodiment, the protein is an antibody. In some embodiments, the antibody is a monoclonal antibody. In some embodiments, the antibody is an antibody against human interleukin-6 (IL-6) receptor. In some embodiments, the antibody is an antibody against human granulocyte-macrophage colony stimulating factor (GM-CSF). In some embodiments, the antibody is an antibody against human CCR5 receptor. In some embodiments, the antibody is antibody against a viral surface antigen, e.g. the spike protein (S protein). In a particular embodiment, the antibody is independently selected from the group consisting of a monoclonal antibody (mAb) such as tocilizumab (e.g., Actemra), leronlimab, TZLS-501, TJM2, sarilumab (e.g., Kevzara), REGN3048 and/or REGN3051.

In an embodiment, the further agent is a plasma-derived agent such as TAK-888. In another embodiment, the further agent is an amniotic fluid concentrate. In another embodiment, the further agent is remestemcel-L (e.g. Ryoncil).

In an embodiment, the further agent alleviates one or more symptoms of an infection of a human with SARS-CoV-1 and/or SARS-CoV-2. Symptoms may include, but are not limited to fever, chest pains, dry cough, dyspnea, headache, and hypoxemia. By co-administered, it is meant simultaneous or sequential administration of anti-IL6 agent and one or more of the further agents defined herein. By further agent it is meant an approved agent, an agent which is in development, or an agent which is said to be useful for the treatment or prevention of infection of a human with SARS-CoV-1 and/or SARS-CoV-2, whichever is being treated or prevented by anti-IL6 agent.

Each of the agents listed above may be specifically combined with an anti-IL6 agent in one of the combination therapies disclosed herein. Each of those combinations is herein specifically individualized.

### Rationale

The present invention is directed to targeting IL6 with an anti-IL6 agent (e.g., anti-IL6 antibody) which may be advantageous over targeting the IL6R for the following reasons: (i) there is less circulating IL6 cytokine versus the receptor (100-1000 fold less) which should allow less frequent dosing and/or a smaller dose of agent (antibody); (ii) the IL-6R has additional ligands which would be inhibited by an anti-IL6R agent (antibody) (CNTF, p28); and (iii) there are polymorphisms in the IL-6R gene, e.g., variations in SNPs that may impact the activity of anti-IL6R agents.

### Clinical Studies

Fourteen interventional studies with administration of sirukumab for the treatment of systemic lupus erythematosus (SLE), cutaneous lupus erythematosus (CLE), lupus nephritis (LN), rheumatoid arthritis (RA), Major Depressive Disorder (MDD) and Giant-cell Arteritis (GCA) have been completed.

As of 23 Oct. 2018, overall, 251 healthy participants and an estimated 5,044 participants with RA, 71 participants with lupus, 193 participants with MDD, and 161 participants with GCA have been exposed in the sirukumab clinical program. Of these, 3,495 participants have received sirukumab (IV or SC) in Janssen-sponsored interventional clinical trials (227 healthy participants, 3,120 participants with RA, 54 participants with lupus, and 94 participants with MDD) and 111 participants have received sirukumab in Glaxo Smith Kline-sponsored clinical trials. Of the total 3,606 participants who have received sirukumab, 272 participants were in the Phase 1 studies, 297 participants were in the Phase 2 studies, and 3,037 participants were in the Phase 3 studies.

Of the participants included in the clinical program, a total of 107 participants received sirukumab IV. Of these, 55 received a dose that was higher than the dose of 5 mg/kg.

In 2016, Janssen Biotech, Inc. filed a Biologics License Application (BLA) with the FDA seeking approval for the use of SC sirukumab in the treatment of RA based upon data from the RA clinical development program. During the review of the BLA, the FDA convened an Advisory Committee meeting, held in 2017. While the Advisory Committee felt that sirukumab convincingly demonstrated efficacy in RA, it could not definitely attribute the imbalance in overall mortality to chance or the study design, and given the availability of other drugs for RA, the Advisory Committee did not recommend approval of sirukumab for RA. As a result, the Sponsor did not pursue further development of SC sirukumab in RA.

### Clinical Efficacy

No clinical efficacy data are available on the use of sirukumab to treat COVID-19 induced ARDS.

### COVID-19 Clinical Trial Plan

TABLE 12
OBJECTIVES AND ENDPOINTS

| | |
|---|---|
| Objectives | Endpoints |

Primary

| | |
|---|---|
| To evaluate the clinical | Time to improvement^{a} of at least 2 |
| response of sirukumab | categories relative to reference on |
| (administered as a single | the 6-point ordinal clinical recovery |
| IV dose) + SOC compared | scale of clinical status (up to Day 28) |
| to placebo + SOC in | |
| confirmed severe or | |
| critical COVID-19 disease | |

Key Secondary

| | |
|---|---|
| To evaluate the clinical | Proportion of participants with an |
| response of sirukumab + | improvement on Day 28 of at least 2 |
| SOC compared to placebo + | categories relative to Baseline on |
| SOC in confirmed severe | the 6-point ordinal clinical recovery |
| or critical COVID-19 | scale |
| disease | |

Other Secondary

| | |
|---|---|
| To evaluate the safety | Incidence of SAEs (up to Day 28) |
| of sirukumab + SOC | Incidence of related AEs (up to |
| compared to placebo + | Day 28) |
| SOC in confirmed severe | Proportion of participants with |
| or critical COVID-19 | severe or life-threatening bacterial, |
| disease | invasive fungal, viral or opportunistic |
| | infections (other than SARS-CoV-2) |
| | (up to Day 28). |
| | Incidence of grade 3 and 4 neutropenia |
| | and lymphocytopenia (up to Day 28) |
| | Incidence of increased ALT >3 × ULN |
| | combined with increased |
| | bilirubin >2 × ULN (up to Day 28) |
| To evaluate the clinical | Time to improvement^{a} of at least 1 |
| response of sirukumab + | category relative to Baseline on the |
| SOC compared to placebo + | 6-point ordinal clinical recovery |
| SOC in confirmed severe | scale (up to Day 28) |
| or critical COVID-19 | Proportion of participants with an |
| disease | improvement on Day 28 of at least 1 |
| | category relative to Baseline on the |
| | 6-point ordinal clinical recovery scale |
| | Time from study intervention to end |
| | of oxygen supplementation (defined as |
| | achieving category 1 or 2 on the 6- |
| | point ordinal clinical recovery scale) |
| | (up to Day 28) |
| | Time from study intervention to |
| | hospital discharge (up to Day 28) |
| | Total length of hospitalization among |
| | the surviving participants (up to Day |
| | 28) |
| | Incidence of all-cause mortality (up |
| | to Day 28) |
| | Number of ventilation free days (up |
| | to Day 28) |
| | Participant's clinical status at Day |
| | 7, 14, 21, 28 (6-point ordinal clinical |
| | recovery scale) |
| | Total time on invasive mechanical |
| | ventilation |
| | Proportion of participants with a |
| | worse category relative to Baseline on |
| | the 6-point ordinal clinical recovery |
| | scale (up to Day 28) |
| | Proportion of participants on |
| | extracorporeal membrane oxygenation |
| | (ECMO) over time |
| | Total time on ECMO |
| To evaluate the safety | Proportion of alive participants at |
| during follow-up | Day 28, Week 8, and Week 16 |
| | Proportion of alive participants that |
| | required readmission at Week 8 and Week |
| | 16 (if previously discharged to home) |
| | Incidence of SAEs up to Week 16 |

Exploratory

| | |
|---|---|
| To evaluate biomarkers | Evaluation including but not limited |
| that may be associated | to IL-6, pro-calcitonin, C-Reactive |
| with response to or | protein (CRP), ferritin, LDH and D- |
| complications of | dimer serum concentrations (through |
| sirukumab treatment | Day 28) |
| To explore changes in | Time to SARS-CoV-2 undetectable viral |
| SARS-CoV-2 viral load | load by RT-PCR (up to Day 28) |
| and viral genome | SARS-CoV-2 viral load over time by |
| | RT-PCR (up to Day 28) |
| | Proportion of participants with |
| | undetectable viral load (at multiple |
| | time points up to Day 28) |
| | Development of SARS-CoV-2 variants |
| To evaluate the incidence | Proportion of participants with SARS- |
| of SARS-CoV-2 viremia | CoV-2 viremia (at multiple time points |
| | up to Day 28) |
| To evaluate pharmacokinetics | Sirukumab serum concentrations |
| following sirukumab 5 | |
| mg/kg treatment | |
| To evaluate immunogenicity | Proportion of participants with |
| following sirukumab | sirukumab antibodies (Day 28 or at |
| treatment | discharge if discharged after Day 28) |
| To evaluate SARS-CoV-2 | Serum levels of SARS-CoV-2 specific |
| humoral immunity | antibodies (IgG and IgM) |

^{a}The improvement should be sustained until Day 28 (or discharge/discontinuation)
^{a}The improvement should be sustained until Day 28 (or discharge/discontinuation).

At the time of data analysis, additional endpoints may be considered for analysis in function of the evolution in scientific knowledge on COVID-19.

Study Design

### Overall Design

This is a randomized, double-blind, placebo-controlled, multicenter, interventional Phase 2 study in hospitalized participants with confirmed severe or critical COVID-19 disease, at risk for progressing to severe ARDS. A target of 270 participants will be randomly assigned in a 2:1 ratio to receive 1 of the following 2 treatments:
Treatment Arm: sirukumab 5 mg/kg IV single dose infusion on Day 1+SOC treatment
Control Arm: placebo W single dose infusion on Day 1+SOC treatment
   Randomization will be stratified by age (<65 and ≥65 years of age) and by use of invasive mechanical ventilation (yes/no) at the time of randomization.

The study will include a Screening Phase (on Day −1), a ‘Day 1 to Day 28’ Phase and Post Day 28 phone calls (Week 8, Week 12 and Week 16). A rapid enrollment of the study is anticipated. The entire study duration for each participant will be 16 Weeks with daily study assessments up to Day 28 or day of discharge (whichever comes first), and phone call assessments thereafter, i.e., at Day 28 in case of discharge prior to Day 28, at Week 8, 12 and 16. The study is considered completed with the completion of the last study assessment (phone call assessment at Week 16) for the last participant in the study or the discontinuation of the last participant in the study, whichever comes last. The impact of a single dose treatment with sirukumab on Day 1+SOC on confirmed COVID-19 severe disease will be evaluated throughout the study.

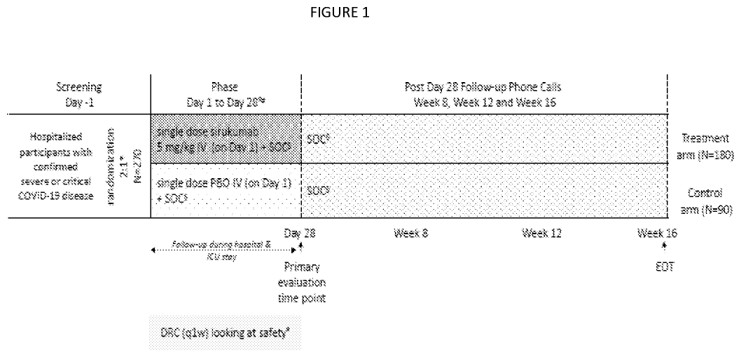

The primary endpoint is the time to improvement of at least 2 categories relative to Baseline on the 6-point ordinal clinical recovery scale (up to 28 days). The improvement should be sustained until Day 28 (or discharge/discontinuation). The primary analysis will be done when all participants reached Day 28 or discontinued earlier. The final analysis will be done when all participants completed the study. A schema diagram of the study design is shown in FIG. 1.

### Blinded, Controlled, Randomized Study

A placebo control will be used to establish the frequency and magnitude of changes in clinical endpoints that may occur in the absence of active intervention with sirukumab and to characterize the safety profile in this placebo group. Randomization will be used to minimize bias in the assignment of participants to treatment arms, to increase the likelihood that known and unknown participant attributes (e.g., demographic and baseline characteristics) are evenly balanced across treatment arms, and to enhance the validity of statistical comparisons across treatment arms. Double blinded intervention will be used to reduce potential bias during data collection and evaluation of clinical endpoints. Stratification will be done by age (<65 and ≥65 years of age) age and by use of invasive mechanical ventilation (yes/no) at randomization.

### Study Population

The goal is to identify a patient population with confirmed severe or critical COVID-19 disease as defined in the Inclusion Criterium 5 (see below) who might benefit most from an anti-IL6 intervention, i.e., patients at risk of progressing to severe ARDS.

### DNA and Biomarker Collection

It is recognized that genetic variation can be an important contributory factor to interindividual differences in response to study intervention and can also serve as a marker for disease susceptibility and prognosis. Pharmacogenomic research may help to explain interindividual variability in clinical outcomes and may help to identify population subgroups that respond differently to an intervention. The goal of the pharmacogenomic component is to evaluate potential genetic associations with prognosis of clinical outcomes in patients and prediction of responsiveness to sirukumab treatment. Biomarker samples will be collected to evaluate the mechanism of action of sirukumab or help to explain interindividual variability in clinical outcomes, to potentially help identify population subgroups that respond differently to an intervention or to SARS-CoV-2 infection and/or to characterize markers associated with SARS-CoV-2 infection. The goal of the biomarker analyses is to evaluate the PD of sirukumab and aid in evaluating the intervention-clinical response relationship. Biomarker samples may be used to help address emerging issues and to enable the development of safer, more effective, and ultimately individualized therapies.

### End of Study Definition

The end of study is considered as the last scheduled study assessment at Week 16 as shown in the Schedule of Activities for the last participant in the study or the discontinuation of the last participant, whichever comes last.

### Study Completion Definition

A participant will be considered to have completed the study if he or she has completed assessments at Week 16 or has experienced a clinical endpoint that precludes further continuation in the study (e.g., early mortality).

### Study Population

Screening for eligible participants will be performed on Day 1. All screening assessments, including obtaining of informed consent and SARS-CoV-2 PCR tests, if positive test is unavailable before screening, should take place within 24 hours prior to randomization. Results from the blood test, pregnancy test, and pulmonary X-ray, completed as standard of care, taken up to 2 days prior to screening will be accepted as screening assessments. SARS-CoV-2 positivity, as determined locally by real time-PCR or any other commercial or public health assay, in any specimen at any time prior to randomization is acceptable and this should not be repeated. If all eligibility criteria are met at screening then randomization may occur on that same day.

### Inclusion Criteria

Each potential participant must satisfy all of the following criteria to be enrolled in the study:

1. Male or female ≥18 and <85 years of age.

### 2. Hospitalized

3. Has laboratory-confirmed SARS-CoV-2 infection as determined by real time-PCR or any other commercial or public health assay, at any time before randomization.
4. Evidence of infiltrates by chest X-ray, chest CT or chest auscultation (rales, crackles).
5. Severe or critical COVID-19 disease, defined as:
Severe disease: Requires supplemental oxygen administration by nasal cannula, simple face mask, or other similar oxygen delivery device (ie, above pre-COVID baseline oxygen requirement, if any, by the participant).
Critical disease: Requires supplemental oxygen delivered by nonrebreather mask or high-flow nasal cannula OR use of non-invasive or invasive ventilation OR requiring treatment in an ICU.
AND at least one of the following:
Requiring supplemental oxygen delivered at a flow ≥6 L/min to sustain a SpO₂>93% regardless of device/route used (corresponds to category 3 or 4 on the 6-point ordinal scale).*

### OR

PaO2/FiO2 ratio <300 mmHg while on invasive mechanical ventilation (with invasive mechanical ventilation for less than 24 hours prior to screening) (corresponds to category 5 on the 6-point ordinal scale).
   *Note: The requirement of supplemental oxygen at ≥6 L/min means that the participant has a SpO₂≤93% on 4 L/min, which constitutes a requirement or a need for at least 6 L/min.
   6. Must understand English language and informed consent must be obtained (or their legally acceptable representative must understand English and sign based on local regulations) indicating that he or she understands the purpose of, and procedures required for, the study and is willing to participate in the study.

### Exclusion Criteria

Any potential participant who meets any of the following criteria will be excluded from participating in the study:

1. On invasive mechanical ventilation for >24 hours at time of screening.
2. Meets local or global criteria to not receive mechanical ventilation or has designated themselves as DNR per a living will.
3. Received an investigational intervention (including investigational vaccines) or used an invasive investigational medical device within 30 days before the planned first dose of study intervention.
Note: the investigator must ensure that the participant is not enrolled in another COVID-19 study with an investigational agent (apart from the exception specified below) prior to completion of Day 28 of the current study.
Exception: participation in a single arm study or compassionate use study is allowed if it is conducted with one of the antiviral drug with demonstrated in vitro-effect against SARS-CoV-2, as mentioned in the CDC guidelines.
4. Currently active, clinically significant cardiovascular abnormalities, such as left ventricular ejection fraction <40% documented by cardiac echo within the previous 12 months, signs of pulmonary embolus, hemodynamic significant pericardial effusion, myocarditis, or Class 3 or 4 congestive heart failure as defined by the New York Heart Association Functional Classification

### AND/OR

Evidence of active cardiac ischemia or history of myocardial infarction, unstable angina or acute coronary syndrome within 6 months prior to randomization.
5. Currently active uncontrolled arrhythmia, any knowledge or evidence of clinically significant arrhythmias (e.g., non-sustained ventricular tachycardia, bradycardia defined as HR less <50 or AV block [Mobitz type II or III], atrial arrhythmia, or any other clinically significant ventricular arrhythmia)
Exception: Participants with premature ventricular contraction, supraventricular tachycardia or new atrial arrhythmias considered to be associated with the underlying disease may be included.
6. Liver function impairment defined as Child Pugh Class B/C based on medical history.
7. Has congenital bleeding diathesis based on medical history.
8. Has a history of severe chronic obstructive pulmonary disease (COPD) or other severe chronic condition (i.e., asthma, cystic fibrosis, fibrotic lung disease) for which the degree of severity is defined as steroid dependent or that requires home oxygen supplementation, supportive non-invasive ventilation, is status/post lung volume reduction surgery (LVRS), or has known forced expiratory volume (FEV)1<50%
9. On renal replacement therapy (defined as peritoneal dialysis or hemodialysis)
10. Screening laboratory test result as follows:
Absolute neutrophil count (ANC)<2.0×10³ cells/μL (SI: <2.0×10⁹ cells/L)
Platelet count <100×10³ cells/μL (SI: <50×10⁹ cells/L)
Estimated glomerular filtration rate (eGFR)≤30 mL/min/1.73 m²
Bilirubin 2×ULN unless bilirubin rise is due to Gilbert's syndrome or of non-hepatic origin
   Prothrombin time (PT)/international normalized ratio (INR) >1.5×ULN and activated partial thromboplastin time (aPTT) >1.5×ULN (unless abnormalities are unrelated to coagulopathy or bleeding disorder).
      11. Pregnant, unless in the opinion of the investigator, the benefit outweighs the risks OR Breastfeeding.
      12. Has active hepatitis B or C infection or HIV/AIDS based on medical history and concomitant medication.
      13. Known active TB, history of incompletely treated TB, suspected or known extrapulmonary TB based on medical history and/or concomitant medication.
      14. Evidence of active bacterial (including but not limited to bacterial pneumonia), fungal, viral or opportunistic infection (other than SARS-CoV-2).
      15. Known allergies, hypersensitivity, or intolerance to sirukumab or its excipients or to other monoclonal antibodies.
      16. Unlikely to be able to complete the study (including but not limited to: likely to be transferred to another hospital, surgery is anticipated to be necessary, in the opinion of the investigator unlikely to survive for >48 hours from screening)
      17. Any condition for which, in the opinion of the investigator, participation would not be in the best interest of the participant (e.g., compromise the well-being) or that could prevent, limit, or confound the protocol-specified assessments.
      18. Taken any disallowed therapies as noted in Section 6.8, Concomitant Therapy before the planned first dose of study intervention.
      19. History of malignancy within 5 years before screening (exceptions are squamous and basal cell carcinomas of the skin and carcinoma in situ of the cervix, or malignancy, which is considered cured with minimal risk of recurrence).
      20. Organ transplant recipient on immunosuppressant therapy

### Description of Interventions

The study intervention will be administered to the participant via an IV infusion using a 5% dextrose W bag with a total volume of 50 mL (5% dextrose). Sirukumab will be injected into the bag for use in the treatment arm, the volume of the bag will be adjusted so that the total bag volume remains 50 mL. No substance will be injected in the bag for the placebo arm, and the 50 mL IV bag will be used as such. An unblinded pharmacist or qualified staff member will prepare the IV bags before distribution to the clinic.

TABLE 13

| | |
|---|---|
| Intervention Name | Sirukumab (CNTO136) |
| Type | Drug |
| Dose Formulation | Solution for infusion |
| Unit Dose Strength(s) | Sirukumab: 100 mg/mL |
| Dosage Level(s) | Sirukumab: 5 mg/kg |
| | single dose |
| Route of Administration | IV infusion |
| Use | Intervention |
| Investigational Medicinal | Yes |
| Product (IMP) | |
| Non-Investigational Medicinal | No |
| Product/Auxiliary Medicinal | |
| Product (NIMP/AxMP) | |
| Sourcing | Provided centrally by |
| | the Sponsor |
| Packaging and Labeling | Each unit will be |
| (Labels will contain information | labeled with unique |
| to meet the applicable regulatory | medication ID number. |
| requirements.) | |
| | Not in child resistant |
| | packaging |
| Delivery Instructions | Refer to IPPI for |
| | instructions on IV |
| | infusion |
| Food/Fasting Requirement | Regardless of food intake |

### Preparation/Handling/Storage/Accountability

### Preparation/Handling/Storage

Sirukumab must be stored at controlled temperatures ranging from 36° F. to 46° F. (2° C. to 8° C.) and protected from light. Sirukumab 5 mg/kg will be administered to the participant via an IV infusion at a rate of not more than 100 mL/min over a time period of approximately 30 minutes using a total volume of 50 mL (5% dextrose). Placebo participants will receive the IV infusion of 50 mL 5% dextrose at the same rate. Aseptic procedures must be used during the study agent infusion.

### Measures to Minimize Bias: Randomization and Blinding

### Intervention Allocation

### Procedures for Randomization and Stratification

Central randomization will be implemented in this study. Participants will be randomly assigned to 1 of 2 intervention groups based on a computer-generated randomization schedule prepared before the study by or under the supervision of the sponsor. The randomization will be balanced by using randomly permuted blocks and will be stratified by age (<65 and ≥65) and invasive mechanical ventilation (yes/no) at randomization. The interactive web response system (IWRS) will assign a unique intervention code, which will dictate the intervention assignment and matching study intervention kit for the participant. The requestor must use his or her own user identification and personal identification number when contacting the IWRS, and will then give the relevant participant details to uniquely identify the participant.

### Blinding

The investigator will not be provided with randomization codes. The codes will be maintained within the IWRS, which has the functionality to allow the investigator to break the blind for an individual participant. Data that may potentially unblind the intervention assignment (i.e., preparation) will be handled with special care to ensure that the integrity of the blind is maintained and the potential for bias is minimized.

The participants, study-site personnel, and investigators will be blinded to treatment allocation throughout the study, except for the designated pharmacist(s) or independent qualified staff member(s) with primary responsibility for study preparation. These unblinded members will not be part of the team performing the evaluations. The infusion administrator will be blinded and can perform other study evaluations. The sponsor study team will be blinded to study treatment allocation until the database release of the primary analysis.

Under normal circumstances, the blind should not be broken until the database for the primary analysis is locked. The investigator may in an emergency determine the identity of the intervention by contacting the IWRS. While the responsibility to break the intervention code in emergency situations resides solely with the investigator, it is recommended that the investigator contact the sponsor or its designee if possible to discuss the particular situation, before breaking the blind. Telephone contact with the sponsor or its designee will be available 24 hours per day, 7 days per week. In the event the blind is broken, the sponsor must be informed as soon as possible. The date and reason for the unblinding must be documented in IWRS, in the appropriate section of the eCRF and in the source document. The documentation received from the IWRS indicating the code break must be retained with the participant's source documents in a secure manner.

Participants who have had their intervention assignment unblinded should continue to return for scheduled evaluations.

In general, randomization codes will be disclosed fully only at the timing of the primary analysis and when the clinical database is closed. However, for the analyses performed for DRC review or for an interim analysis, the randomization codes and, if required, the translation of randomization codes into intervention and control groups will be disclosed to those authorized and only for those participants included in the DRC or interim analysis.

### Prestudy and Concomitant Therapy

Sirukumab has immunomodulatory effects that may predispose participants to opportunistic infections. Therefore, all participants are to be managed according to local treatment guidelines, including the latest version of CDC Information for Clinicians on Therapeutic Options for Patients with COVID-19. Open-label or off-label use of agents that are intended to inhibit SARS-Cov-2 viral activity are permitted in the study, but they must be listed in the CDC guidelines on Therapeutic Options for patients with COVID-19.

### Disallowed Concomitant Medications

Conventional synthetic disease-modifying anti-rheumatic drugs/immunosuppressive agents:
   Oral anti-rejection or immunomodulatory drugs (including toclizumab) are disallowed from 6 months prior to randomization until the end of the study.
   Treatment with other anti-IL-6, anti-IL6R antagonists, Janus kinase inhibitors, ustekinumab (anti IL-12/23), or anti IL-23 agents (guselkumab) is disallowed within 5 half-lives (Table 15) or from 30 days prior to randomization until the end of the study (whichever comes first).

TABLE 15
Treatment Half-life and 5 Half-lives

| | | |
|---|---|---|
| Drug | Half-life | 5 half-lives |

| | | | | |
|---|---|---|---|---|
| toclizumab IV | 21.5 | days | 107.5 | days |
| siltuximab IV | 20.6 | days | 103 | days |
| sarilumab IV 200 mg | 10 | days | 50 | days |
| sarilumab IV 150 mg | 8 | days | 40 | days |
| ustekinumab IV | 19 | days | 95 | days |
| guselkumab IV | 15-18 | days | 75-90 | days |
| ustekinumab SC | 14.9-45.6 | days | 74.5-228 | days |
| upadacitinib | 8-14 | hours | 40-70 | hours |
| baricitinib | 12 | hours | 60 | hours |
| tofacitinib XR | 6-8 | hours | 30-40 | hours |

Source: Respective US Prescribing Informations

Systemic treatment with disease-modifying anti-rheumatic drugs or immunosuppressive agents including methotrexate, bucillamine, azathioprine, oral cyclosporine A, tacrolimus, mycophenolate mofetil, leflunomide, oral or parenteral gold, and IL-1ra (anakinra) is disallowed from 2 weeks prior to randomization until the end of the study.
Exceptions: sulfasalazine, hydroxychloroquine and chloroquine
The use of cyclophosphamide is disallowed from 12 weeks prior to randomization until the end of the study.
Corticosteroids:
   potential participants on chronic (for >3 months in duration) prednisone in a dose higher than 10 mg/day or other oral corticosteroids at an equivalent dose for a non-COPD-related condition are not eligible for the study
   potential participants using oral corticosteroids for a non-COVID-19-related condition are not eligible for the study.
The use of leflunomide is disallowed from 8 weeks prior to randomization until the end of the study. Potential participants who have undergone standard cholestyramine washout may qualify if it is done at least 4 weeks before randomization: cholestyramine at a dosage of 8 mg/3 times a day for at least 24 hours, or activated charcoal at a dosage of 50 mg/4 times a day for at least 24 hours.
The participants should not have received an investigational intervention (including investigational vaccines) or used an invasive investigational medical device within 30 days of the study medication (whichever is longer) before the planned first dose of study intervention and should not receive any investigational medication, other than sirukumab, prior to completion of Day 28.
   Note: the investigator must ensure that the participant is not enrolled in another COVID-19 study with an investigational intervention (apart from the exception specified below) prior to completion of Day 28 of the current study.
   Exception: participation to a single arm study or compassionate use study, this is allowed if it is conducted with one of the antiviral drug with demonstrated in vitro-effect against SARS-CoV-2, as mentioned in the CDC guidelines.
   The sponsor must be notified in advance (or as soon as possible thereafter) of any instances in which prohibited therapies are administered.
   Medications to be Used with Caution as their Exposure could be Modified by the Study Medication

Examples include but are not limited to warfarin, theophylline, digoxin, antiepileptics, antiarrhythmics.

Various in vitro studies have shown that cytokines such as IL-6 can affect the expression and activity of multiple cytochrome P450 (CYP) enzymes, such as CYP3A4, CYP2C9, CYP2C19, and CYP1A2. Sirukumab treatment may potentially reverse the effect of IL-6 on CYP enzyme activities in patients with elevated IL-6, which could lead to altered metabolism of drugs that are CYP substrates. The effect of sirukumab on the CYP enzymes may be clinically relevant for CYP substrates with a narrow therapeutic index, where the dose is individually adjusted. Therefore, it is recommended to use these type of drugs with caution until 12 weeks after sirukumab administration (3× half-life); therapeutic monitoring of effect (e.g., warfarin) or drug concentration (e.g., theophylline) should be performed and the individual dose of the drug adjusted as needed. Caution should be exercised when sirukumab is co-administered with CYP3A4 substrate drugs where a decrease in effectiveness would be undesirable (e.g., oral contraceptives). The effect of sirukumab on CYP enzymes may persist for several weeks after stopping therapy. Therefore, female participants using oral contraceptives should use an additional contraceptive method. Live vaccines should not be given during the study.

### Data Collection of Concomitant Medications

To the extent possible, prestudy therapies administered up to 30 days before the study intervention must be recorded at screening. Concomitant therapies must be recorded in the CRF throughout the study beginning with the dose of study intervention to 16 Weeks after the study intervention. Recorded information will include a description of the type of therapy, duration of use, dosing regimen, route of administration, and indication. Modification of an effective preexisting therapy should not be made for the explicit purpose of entering a participant into the study.

### Overview

The Schedule of Activities (SoA) summarizes the frequency and timing of respiratory function assessments, virology assessments, pharmacology assessments, exploratory biomarkers/pharmacogenomics, and safety measurements applicable to this study. All screening assessments should take place prior to randomization and all baseline assessments prior to study intervention administration. Results from the blood test, pregnancy test, and pulmonary X-ray, completed as standard of care, taken up to 2 days prior to screening will be accepted for screening assessments. SARS-CoV-2 positivity, as determined by real time-PCR or any other commercial or public health assay, in any specimen at any time prior to randomization is acceptable and this should not be repeated. All screening/baseline assessments should take place prior to randomization and/or study intervention administration. If multiple assessments are scheduled for the same timepoint, it is recommended that procedures be performed in the following sequence: ECG, oxygen saturation, vital signs, blood sampling Blood collections for PK assessments should be kept as close to the specified time as possible. Other measurements may be done earlier than specified timepoints if needed. Actual dates and times of assessments will be recorded in the source documentation and CRF. The amount of blood drawn from each participant in this study is approximately 275 mL. Repeat or unscheduled samples may be taken for safety reasons or for technical issues with the samples.

### Sample Collection and Handling

The actual dates and times of sample collection must be recorded in the CRF or laboratory requisition form. Refer to the SoA for the timing and frequency of all sample collections. Instructions for the collection, handling, storage, and shipment of samples are found in the laboratory manual that will be provided. Collection, handling, storage, and shipment of samples must be under the specified, and where applicable, controlled temperature conditions as indicated in the laboratory manual.

### Efficacy Assessments

Efficacy assessments will be done per the SoA and will include all information necessary for the 6-point ordinal Clinical Recovery Scale, level of consciousness (Glasgow coma score [GCS]), virology assessment, supplemental oxygen use, resting SpO₂, arterial blood gas results, and pulmonary X-ray. Details for selected assessments are provided below.

### Six-Point Ordinal Clinical Recovery Scale

The 6-point ordinal clinical recovery scale provides 6 mutually exclusive conditions ordered from best to worst, and the score reflects the participant's worst situation on the day assessed. The sponsor will assign the ordinal scale category based on the assessment of the participant's clinical status by the investigator. The ordinal clinical recovery scale categories are defined below.

### Ordinal Clinical Recovery Scale (and Definitions)

### 1. Not Hospitalized

Any of the following:
Discharged from the hospital the day of assessment
Hospitalized at the day of assessment but ready for discharge on the day of assessment, as judged by the investigator (e.g., if still hospitalized in case of lack of bed availability in a skilled nursing facility, lack of social support at home).
   2. Hospitalization, not requiring supplemental oxygen
   3. Hospitalized, requiring supplemental oxygen
Hospitalized on the day of assessment (including readmittance), and supplemental oxygen is required by the participant

Requiring supplemental oxygen is defined by:
Receiving supplemental oxygen through a face mask or nasal cannula and not being able to sustain a blood oxygen saturation of >93% when breathing room air.
OR
Not receiving supplemental oxygen and having a blood oxygen saturation of ≤93% sustained for 5 minutes.
   4. Hospitalized, on non-invasive pressure ventilation or high flow oxygen devices
On high flow oxygen nasal canula.
On continuous positive airway pressure (CPAP)/bilevel positive airway pressure (BIPAP).
On nonrebreather device.
On supplemental oxygen with a FiO2 (worst of the day) of 50% or higher unless they satisfy a higher category (eg, on invasive mechanical ventilation).
   5. Hospitalized, on invasive mechanical ventilation or ECMO
Any oxygen support requiring intubation or extracorporeal oxygenation
Invasive mechanical ventilation is used at any time on the day of assessment.

### 6. Death

Participant died at any time on the day of assessment or earlier (all-cause mortality).
   In addition, the site will be asked whether or not they are working under (a) restricted material resources for supplemental oxygenation/ventilation and/or (b) changes in hospital discharge policies. Finally, a second 6-point ordinal clinical recovery scale is to be filled-in, with the assumption of working under unrestricted resources and conditions. The frequency and timing of the assessment can be found in the SoA.

### Level of Consciousness

The participant's level of consciousness will be assessed using the Glasgow Come Scale (GCS). The GCS is a neurological scale ranging from 3 (lowest, corresponds to deep coma or death) to 15 (highest, corresponds to a fully awake person) to assess the state of a person's consciousness. The central nervous system (CNS) score ranges from 15 (Score 0) to <6 (Score 4). The investigator should record the worst total GCS score once a day in the eCRF, as long as the patient is in ICU.

The level of sedation of the participant will be derived from the type of medication entered for indication sedation on the Concomitant Medication page of the eCRF. The frequency and timing of the assessment can be found in the SoA.

### Virology Assessments

### Respiratory Tract Samples

SARS-CoV-2 positivity should be documented based on local testing on any specimen, by RT-PCR or any other commercial or public health assay, any time before randomization. This might require a local test using a NP swab obtained at screening

In addition, nasopharyngeal swabs will be collected for central testing. A NP swab will be used to collect secretions from patients to explore quantification of viral load of SARS-CoV-2 virus. At baseline, the presence of other respiratory pathogens, using multiplex PCR, will also be tested. Furthermore, sequencing might be performed upon request of the virologist to determine mutations in the viral genome.

For participant who are intubated, endotracheal samples need to be taken at the same time as the NP swabs. If taking both samples is not feasible, the NP should be given priority. Collected samples need to be sent to the central lab to explore quantification of viral load. Sequencing might be performed upon request of the virologist to determine mutations in the viral genome.

Only one sample should be collected if the NP sample for detection and the NP sample for quantification are collected on the same day. This sample should be aliquoted and the remaining aliquots of the NP samples should be stored and sent to the central lab for quantification of SARS-CoV-2. For each participant, NP sampling should be done at approximately the same time (±4 hours) on each sampling day and from the same nostril. Leftover NP swabs and endotracheal samples may be used for exploratory biomarker analyses. If viral RNA is detected in nasopharyngeal samples at day of discharge, all possible efforts will be made to follow-up subjects and collect samples until viral RNA is negative, considering the current pandemic and related logistical challenges.

The frequency and timing of the assessments can be found in the SoA. Details about sample collection, processing, and shipping will be provided in the laboratory manual.

### Plasma Samples

In addition to NP swabs, plasma samples will be collected to assess SARS-CoV-2 viremia. Leftover plasma samples may be used for exploratory biomarker analyses. The frequency and timing of the assessments can be found in the SoA. Details about sample collection, processing, and shipping will be provided in the laboratory manual.

### Stool Samples (Optional)

In addition to respiratory tract and plasma samples, stool samples will be collected to explore quantification of viral load of SARS-CoV-2 virus. The stool collections are optional and will only be collected if feasible for the site. Collected samples need to be sent to the central lab. Leftover stool samples may be used for exploratory biomarker analyses. The frequency and timing of the assessments can be found in the SoA. Details about sample collection, processing, and shipping will be provided in the laboratory manual.

### Supplemental Oxygen Use

Supplemental oxygen/percentage of inspired oxygen (FiO₂) use (if any) will be measured to monitor the patient's status regarding gas exchange as applicable. The following will be recorded:
Oxygen delivery device (eg, nasal cannula, simple face mask, non-rebreather mask, high flow nasal cannula, non-invasive ventilation, invasive mechanical ventilation, extracorporeal life support, etc.)
Oxygen flow rate in liters/min
Record FiO₂ and SpO₂ data 4 times per day, and at any time of blood gas measurements. Record values that are sustained for at least 1 hour. For the analyses, the worst values sustained for at least 1 hour, on the highest level of oxygen supplementation method of the day, will be used
If a patient is using more than one device (e.g., extracorporeal life support and invasive ventilation), information (the worst recording) from both devices will be recorded separately
If a patient does not need oxygen supplement, this should also be recorded
   The frequency and timing of the assessment can be found in the SoA.

### Resting SpO₂

Resting SpO₂ will be measured to assess arterial oxyhemoglobin saturation. SpO₂ will be measured using a fingertip or similar non-invasive device, while patient is stable, following 5 minutes of rest (inactivity) in supine, semi-recumbent, or sitting position and will only be measured in the presence of a good SpO₂ wave form. SpO₂ must be measured simultaneously with recorded supplemental oxygen/FiO₂ data. For participants receiving invasive or non-invasive mechanical ventilation, peripheral oxygen saturation should be measured with the ventilatory support in place, and it should be recorded. The frequency and timing of the assessment can be found in the SoA.

### Arterial Blood Gas Test

An arterial blood test will be conducted to assess the following parameters:
pH: acid-base balance of blood
PaO₂: partial pressure of oxygen in arterial blood
PaCO₂: partial pressure of carbon dioxide in arterial blood
SaO₂: arterial oxygen saturation

Results should be recorded in arterial blood gas results electronic clinical report form (eCRF). The frequency and timing of the assessment can be found in the SoA.

### Safety Assessments

Safety and tolerability will be evaluated throughout the study from obtaining confirmed consent onwards until the last study-related activity. Adverse events will be reported and followed by the investigator. Any clinically significant abnormalities persisting at the end of the study/early withdrawal will be followed by the investigator until resolution or until a clinically stable condition is reached. The study will include the following evaluations of safety and tolerability according to the time points provided in the SoA.

### Physical Examinations

A targeted physical examination will be performed as indicated in the SoA. A targeted physical examination includes lung auscultation and any examination as indicated by the patient's medical history. Height and body weight are only to be measured at screening if not already available in the participant's chart and if practically feasible. Clinically significant findings should be documented as AE in the eCRF if they are serious, possibly related to the study drug or correspond to an adverse event of interest.

### Vital Signs

Temperature, pulse rate, respiratory rate, and blood pressure (SBP/DBP) will be assessed. The frequency and timing for each assessment can be found in the SoA. Body temperature will be measured according to local hospital protocols and according to the manufacturer's instructions for use of the device. Body temperature should be measured using the same method each time: temperature should be measured after at least 5 minutes of rest (supine or sitting) and before taking antipyretics or more than 4 hours after the last dose of antipyretics. Blood pressure and pulse/heart rate measurements will be assessed with a completely automated device. Manual techniques will be used only if an automated device is not available. Confirmatory vital signs measurements can be performed if inconsistent with a prior measurement.

Any clinically relevant abnormalities or changes in severity should be documented as AE in the eCRF if they are serious, possibly related to the study drug or correspond to an adverse event of interest.

### Electrocardiograms

Participants should rest in a supine position for at least 5 minutes before ECG collection and should refrain from talking or moving arms or legs. If multiple assessments are scheduled for the same time point as ECG recording, the procedures should preferably be performed in the following order: ECG(s), oxygen saturation, vital signs, blood draw. Heart rate will be recorded from the ventricular rate and the PR, QRS, and QT (identify QT interval corrected for heart rate [QTc] using Bazett's formula [QTcB] or QTc using Fridericia's formula [QTcF]) intervals will be recorded in the eCRF. The ECG strips or reports will be retained with the source. The frequency and timing for each assessment can be found in the SoA. Any clinically relevant abnormalities or changes in severity should be documented as AE in the eCRF if they are serious, possibly related to the study drug or correspond to an adverse event of interest.

### Clinical Safety Laboratory Assessments

Blood samples for serum chemistry and hematology will be collected. The investigator must review the laboratory results, document this review, and record any clinically relevant changes occurring during the study in the AE section of the CRF. The laboratory reports must be filed with the source documents. Any clinically relevant abnormalities or changes in severity should be documented as AE in the eCRF if they are serious, possibly related to the study drug or correspond to an adverse event of special interest (AESI).

### Pregnancy Testing

At screening, absence of pregnancy in women of childbearing potential should preferably be confirmed by a negative highly sensitive serum (□-human chorionic gonadotropin [□hCG]) test. The result of a prior serum pregnancy test that occurred within 2 calendar days (as part of SOC) before obtaining consent can be used in lieu of the screening requirement. If the timeframe does not allow for results of a serum test (in emergency situations), a urine rapid pregnancy test is acceptable.

### Vital Status

During the post Day 28 follow-up phone call visits vital status of study participants will be recorded. If the participant is deceased, date and cause of mortality should be recorded. Death should be documented as SAE in the eCRF.

### Adverse Events, Serious Adverse Events, and Other Safety Reporting

Timely, accurate, and complete reporting and analysis of safety information, including AEs, SAEs, and product quality complaints (PQCs), from clinical studies are crucial for the protection of participants, investigators, and the sponsor, and are mandated by regulatory agencies worldwide. The sponsor has established Standard Operating Procedures in conformity with regulatory requirements worldwide to ensure appropriate reporting of safety information; all clinical studies conducted by the sponsor or its affiliates will be conducted in accordance with those procedures. Adverse events will be reported by the participant (or, when appropriate, by a caregiver, surrogate, or the participant's legally acceptable representative) for the duration of the study.

### Time Period and Frequency for Collecting Adverse Event and Serious Adverse Event Information

All Adverse Events

All adverse events and special reporting situations, whether serious or non-serious, will be reported from the time a signed and dated ICF is obtained until completion of the participant's last study-related procedure, which may include phone call contact for follow-up of safety. SAEs, including those spontaneously reported to the investigator within 30 days after the last dose of study intervention, must be reported using the SAE Form. The sponsor will evaluate any safety information that is spontaneously reported by an investigator beyond the time frame specified in the protocol.

### Serious Adverse Events

All SAEs, as well as PQCs, occurring during the study must be reported to the appropriate sponsor contact person by study-site personnel within 24 hours of their knowledge of the event. Serious adverse events, including those spontaneously reported to the investigator within 30 days after the EOT (Week 16) phone call visit, must be reported using a Serious Adverse Event form. The sponsor will evaluate any safety information that is spontaneously reported by an investigator beyond the time frame specified in the protocol.

Information regarding SAEs will be transmitted to the sponsor using the Serious Adverse Event Form and Safety Report Form of the CRF, which must be completed and reviewed by a physician from the study site, and transmitted to the sponsor within 24 hours. The initial and follow-up reports of an SAE should be transmitted electronically or by facsimile (fax). Telephone reporting should be the exception and the reporter should be asked to complete the appropriate form(s) first.

### Method of Detecting Adverse Events and Serious Adverse Events

Care will be taken not to introduce bias when detecting AEs or SAEs. Open-ended and nonleading verbal questioning of the participant is the preferred method to inquire about adverse event occurrence. During the hospital stay, in case participants are non-responsive, investigators will report AEs as specified.

### Solicited Adverse Events

Solicited adverse events are predefined local (at the injection site) and systemic events for which the participant is specifically questioned.

### Unsolicited Adverse Events

Unsolicited AEs are all AEs for which the participant is not specifically questioned.

### Follow-Up of Adverse Events and Serious Adverse Events

The investigator is obligated to perform or arrange for the conduct of supplemental measurements and evaluations as medically indicated to elucidate the nature and causality of the AE, SAE, or PQC as fully as possible. This may include additional laboratory tests or investigations, histopathological examinations, or consultation with other health care professionals.

### Regulatory Reporting Requirements for Serious Adverse Events

The sponsor assumes responsibility for appropriate reporting of AEs to the regulatory authorities. The sponsor will also report to the investigator (and the head of the investigational institute where required) all suspected unexpected serious adverse reactions (SUSARs). The investigator (or sponsor where required) must report SUSARs to the appropriate Independent Ethics Committee/Institutional Review Board (IEC/IRB) that approved the protocol unless otherwise required and documented by the IEC/IRB. A SUSAR will be reported to regulatory authorities unblinded. Participating investigators and IEC/IRB will receive a blinded SUSAR summary, unless otherwise specified.

### Pregnancy

All initial reports of pregnancy in female participants or partners of male participants must be reported to the sponsor by the study-site personnel within 24 hours of their knowledge of the event using the appropriate pregnancy notification form. Abnormal pregnancy outcomes (e.g., spontaneous abortion, fetal death, stillbirth, congenital anomalies, ectopic pregnancy) are considered serious adverse events and must be reported using a serious adverse event reporting form. Follow-up information regarding the outcome of the pregnancy and any postnatal sequelae in the infant will be required.

### Adverse Events of Special Interest

Adverse events of special interest for the single IV administration of sirukumab (5 mg/kg) are provided below.

TABLE 10
Events of Interest for a Single IV Dose of Interest

| Potential Risks of | | |
|---|---|---|
| Clinical Significance | Summary of Data/Rationale | Mitigation Strategy |
| Serious infections | Interleukin-6 stimulateshepatic acute- | Inclusion and exclusion |
| (Bacterial, fungal, | phase proteins and Ig production and | criteria; hematologic |
| and viral infection) | promotes the growth and differentiation | monitoring; safety evaluation; |
| | of T cells, B cells, and tumor cells. | and supportive treatment. |
| | Interleukin-6 is a mediator of | |
| | inflammation and cellular immune | |
| | responses in the defense against some | |
| | intracellular pathogens. Interleukin-6 | |
| | has been known to serve as a marker | |
| | of disease severity for infections. | |
| | Although IL-6 modulates certain | |
| | physiologic acute-phase responses to | |
| | infection, a complete lack of IL-6 | |
| | has not been shown to alter mortality | |
| | rates in IL-6 knockout mice. Blockage | |
| | of IL-6 may blunt the acute-phase | |
| | pyretic response and therefore, fever | |
| | may be masked in participants receiving | |
| | sirukumab. Serious, life-threatening | |
| | infections such as septic shock, some | |
| | of which have been fatal, have occurred | |
| | in participants receiving sirukumab. | |
| Hypersensitivity | Reactions observed in humans after | Inclusion and exclusion |
| | IV or SC administration of mAbs | criteria; clinical monitoring; |
| | include headache, fever, facial | safety evaluation; and supportive |
| | flushing, pruritus, urticaria, | treatment. |
| | dermatitis, myalgia, nausea, chest | |
| | tightness, laryngeal edema, dyspnea, | |
| | vomiting, erythema, abdominal | |
| | discomfort, diaphoresis, shivers, | |
| | hypertension, Stevens- Johnson | |
| | syndrome, lightheadedness, hypotension, | |
| | palpitations, and somnolence. | |
| Hematologic Events | Neutropenia and thrombocytopenia | Inclusion and exclusion |
| | have occurred in sirukumab studies, | criteria; clinical monitoring; |
| | including severe thrombocytopenia | safety evaluation; and |
| | associated with bleeding. In the | supportive treatment. |
| | Phase 2 and Phase 3 RA studies, | Hematology results should be |
| | decreases in ANC and platelets | reported to the sponsor via |
| | occurred in all sirukumab treatment | eCRF data entry approximately |
| | groups. Most patients who developed | 72 hours from results becoming |
| | neutropenia while being treated | available. |
| | with sirukumab did not develop | |
| | infections, and most patients who | |
| | developed thrombocytopenia did not | |
| | report bleeding events. Changes | |
| | were not observed in participants | |
| | who received placebo, but did | |
| | occur after participants crossed | |
| | over to active treatment. No dose | |
| | response was observed. | |
| Liver enzymes | Increases (1 to 3 × ULN, | Monitoring of liver |
| | sometimes >5 × ULN) in blood ALT | parameters (ALT, AST, |
| | and AST values were observed in | bilirubin [total, direct |
| | participants in completed and | and indirect], coagulation |
| | ongoing studies of sirukumab; the | factors, alkaline phosphatase) |
| | majority were transient, asymptomatic, | Liver parameter results |
| | and not associated with an increase | should be reported to the |
| | in bilirubin | sponsor via eCRF data entry |
| | | approximately 72 hours from |
| | | results becoming available. |

### Pharmacokinetics and Immunogenicity

Serum samples will be used to evaluate the pharmacokinetics of sirukumab, IL-6, as well as antibodies to sirukumab. Serum collected for pharmacokinetic and immunogenicity analyses may additionally be used to evaluate biomarkers, safety or efficacy aspects that address concerns arising during or after the study period. Genetic analyses will not be performed on these serum samples. Participant confidentiality will be maintained.

### Evaluations

At visits where PK, immunogenicity, and IL-6 will be evaluated (Day 1 predose, Day 28, and additionally on the day of discharge if participants are hospitalized >28 days), one venous blood draw will be collected as specified in the SoA. Serum samples will be obtained and split into 3 aliquots (one for sirukumab concentration, one for antibodies to sirukumab, and one for IL-6). At visits where PK ad IL-6 will be evaluated (Day 1 postdose, Day 14, Day 21), one venous blood draw will be collected as specified in the SoA. Serum samples will be obtained and split into 2 aliquots (one for sirukumab concentration and one for IL-6).

### Analytical Procedures

### Pharmacokinetics

Serum samples will be analyzed to determine concentrations of sirukumab using a validated, specific, and sensitive immunoassay method by or under the supervision of the sponsor.

### Immunogenicity

The detection and characterization of antibodies to sirukumab will be performed using a validated immunoassay method by or under the supervision of the sponsor.

### Pharmacokinetic Parameters and Evaluations

If feasible, a population PK approach will be used to characterize the disposition characteristics of sirukumab. Total systemic clearance (CL) and volume of distribution (V) after IV administration may be estimated from population PK modeling using nonlinear mixed effects model (NONMEM) approach.

### Immunogenicity Assessments

Antibodies to sirukumab will be evaluated in serum samples collected on Day 1 predose, Day 28, and additionally on the day of discharge if participants are hospitalized longer than 28 days. Serum samples will be screened for antibodies binding to sirukumab and the titer of confirmed positive samples will be reported. Other analyses may be performed to verify the stability of antibodies to sirukumab and/or further characterize the immunogenicity of sirukumab.

### Genetics and Pharmacogenomics

An optional pharmacogenomic (host DNA) blood sample may be collected (preferably at baseline) to allow for host pharmacogenomic research, where local regulations permit. Pharmacogenomic research may include expression quantitative trait locus (eQTL) mapping, single-nucleotide polymorphisms (SNPs) mapping and whole genome sequencing that is related to the study intervention and/or SARS-CoV-2 infection. Participant participation in pharmacogenomic research is optional.

### Biomarkers

The frequency and timing of the assessment can be found in the SoA

### Biomarker Sample Collection for Exploratory Endpoint Evaluation

Blood samples will be collected to evaluate biomarkers that may be associated with the safety, efficacy and PK of sirukumab and/or with SARS-CoV-2 infection. Evaluations may include, but are not limited to, IL-6, pro-calcitonin, CRP, ferritin, LDH, and D-dimer concentrations. In addition, humoral immunity to SARS-CoV-2 will be evaluated by measuring SARS-CoV-2 specific antibodies (IgG and IgM).

### Biomarker Sample Collection for Research

The study includes collection of blood samples for exploratory analysis of host biomarkers at the host RNA, protein and cell level. Samples will only be obtained and used if not restricted by local regulations. Samples can only be used for research related to sirukumab or SARS-CoV-2 infection and/or to develop tests/assays related to sirukumab or SARS-CoV-2 infection. This may include target pathway of IL-6 inhibition, and the impact on pneumonia and respiratory illness associated with SARS-CoV-2 infection. Blood samples will be taken at the time points indicated in the SoA. These samples can be used to explore the emergence of antibodies to sirukumab (antidrug antibodies) and/or emergence of antibodies to SARS-CoV-2. Analysis of exploratory biomarkers may be conducted and may be reported separately from this study. These samples may be stored for up to 15 years after the study completion.

### Statistical Considerations

Statistical analysis is planned. A general description of the statistical methods to be used to analyze the efficacy and safety data is outlined below.

### Sample Size Determination

The study will aim to enroll approximately 270 participants, with 180 participants in the sirukumab treatment arm and 90 participants in the control arm.

For the sample size determination for the primary endpoint (time to improvement of at least 2 categories relative to Baseline on the 6-point ordinal clinical recovery scale), it is assumed that the log transformed time to improvement (days) in survivors in the control arm follows a normal distribution with mean of log 14 and a standard deviation 0.47. Participants who die prior to Day 28 are treated as right censored at Day 28. The mortality in the control arm is assumed to be 50% by Day 28. Assuming a reduction in mortality from 50% to 35% (30% relative reduction) and a reduction of the median time to clinical improvement from 14 days to 10.5 days (25% reduction) in the surviving participants, at least 150 participants are required to have at least 80% power for a Gehan-Wilcoxon test, at a significance level of 5% two-sided.

The proportion of participants with a clinical improvement of at least 2 categories at Day 28 is a key secondary endpoint. In order to have 80% power to demonstrate also a significant difference between the treatment groups for this endpoint (46% vs. 64% derived from the above assumptions), the study sample size will be 270. This will increase the power for the primary endpoint to 96%.

### Populations for Analysis Sets

For purposes of analysis, the following populations are defined:

The efficacy endpoints will be analyzed on the Intent-to-Treat-infected (ITT-i) and by actual treatment received. The ITT-i set consists of all participants who were randomized and treated and were confirmed to have SARS-CoV-2 infection. All safety endpoints will be evaluated on the Safety population, consisting of all participants who received at least one dose of study drug and will be analyzed by treatment arm as treated. Pharmacokinetic data will be evaluated on participants in the ITT-i set who received sirukumab.

### Statistical Analyses

The statistical analysis plan will be finalized prior to database lock of the primary analysis and it will include a more technical and detailed description of the statistical analyses described in this section. This section is a summary of the planned statistical analyses of the most important endpoints including primary and key secondary endpoints. The primary analysis will be done when all participants reached Day 28 or discontinued earlier. The final analysis will be done when all participants completed the study. Interim analyses might be performed depending on the enrollment rate to guide further development and to support interactions with Health Authorities.

### General Considerations

For all participants who receive study drug descriptive statistics will be provided. All demographic characteristics (e.g., age, race, ethnicity, height, body weight, body mass index) and other initial participant characteristics (e.g., physical examination, medical and surgical history, concomitant diseases) will be tabulated and analyzed descriptively. Subgroup analyses will be performed based on the stratification factors (age [<65 and ≥65 years of age] and use of invasive mechanical ventilation [yes/no]) a selection of the major baseline parameters.

### Primary Endpoint(s)

The primary efficacy analysis will be based on the ITT-i analysis set and the primary efficacy endpoint is the ‘time to improvement of at least 2 categories relative to Base line on a 6-point ordinal clinical recovery scale. The improvement should be sustained until Day 28 (or discharge/discontinuation). Time to clinical improvement will be assessed during the 28-day period after study drug administration, with failure to reach clinical improvement or death before Day 28 considered as right-censored at Day 28. This primary parameter will be analyzed by a stratified Gehan-Wilcoxon test (using the stratification factors). Kaplan-Meier curves, overall and by stratum will be used to graphically present the primary parameter. As sensitivity analyses, a stratified log-rank test and a Cox proportional hazards model will also be applied.

First, the primary endpoint will be tested for superiority of sirukumab over placebo at the 2-sided 5% significance level. If superiority is shown on the primary endpoint, then the key secondary endpoint will also be tested at the same significance level. All other statistical tests will be done as exploratory.

### Secondary Endpoint

All secondary endpoints will be analyzed graphically and descriptively as described in the statistical analysis plan. For continuous variables, descriptive statistics (n, mean, SD, median, minimum, maximum, and 95% confidence intervals [M]) will be calculated. For categorical variables, frequency tables and corresponding 95% CIs will be presented.

### Key Secondary Endpoint

The proportion of participants with an improvement on Day 28 of at least 2 categories on the 6-point ordinal clinical recovery scale relative to Baseline is a key secondary endpoint. This parameter will be analyzed using a logistic regression model including the stratification factors.

### Other Secondary Endpoints

To compare the proportion of participants with an improvement on Day 28 of at least 1 category on the 6-point ordinal clinical recovery scale relative to Baseline, proportion of participants with a worse score on the 6-point ordinal clinical recovery scale relative to Baseline, mortality rates, other ordinal endpoints, and proportion of participants on ECMO, a logistic regression model will be used. Stratification factors will be added to the model.

In addition, the following statistical testing will be done: Total length of hospitalization, total time on invasive mechanical ventilation, number of ventilation free days, and total time on ECMO will be analyzed by the stratified Wilcoxon Rank-Sum test and using the stratification factors. Corresponding 95% CIs will be derived using the Hodges-Lehmann approach. Time to improvement of at least 2 categories and other ‘time to event’ parameters will be analyzed using the stratified Gehan-Wilcoxon test. A proportional odds model will be used to analyze the 6-point ordinal clinical recovery scale of the clinical status on each day up to Day 28. Changes in the SOC after treatment administration will be tabulated by treatment group. Other secondary parameters might be added in the SAP.

### Exploratory Endpoint(s)

Time to viral negativity will be analyzed analogously to that of the primary efficacy parameter. SARS-CoV-2 viral load in NP swabs will be measured by a qRT-PCR assay. These data will be analyzed graphically and descriptively as described in the statistical analysis plan. In addition, the following statistical testing will be done: Total length of hospitalization, total time on invasive mechanical ventilation, number of ventilation free days, and duration of ECMO will be analyzed by the stratified Wilcoxon Rank-Sum test and using the stratification factors. Corresponding 95% CIs will be derived using the Hodges-Lehmann approach. To compare the mortality rates and proportion of participants with a worse score compared to drug intervention, a logistic regression model will be used. Stratification factors will be added to the model. Time to improvement of at least 2 categories and other ‘time to event’ parameters will be analyzed using the stratified Gehan-Wilcoxon test. A proportional odds model will be used to analyze the 6-point ordinal clinical recovery scale of the clinical status on each day up to Day 28. Other secondary parameters might be added in the SAP.

### Key Exploratory Endpoint(s)

Time to viral negativity will be analyzed analogously to that of the primary efficacy parameter. SARS-CoV-2 viral load in NP swabs will be measured by a qRT-PCR assay. These data will be analyzed graphically and descriptively as described in the statistical analysis plan. Other exploratory parameters might be added in the SAP.

### Safety Analyses

All safety analyses will be made on the Safety Population.

### Adverse Events

The verbatim terms used in the CRF by investigators to identify adverse events will be coded using the Medical Dictionary for Regulatory Activities (MedDRA). Any AE occurring at or after the initial administration of study intervention is considered to be treatment-emergent. All reported AEs will be included in the analysis. For each AE, the percentage of participants who experience at least 1 occurrence of the given event will be summarized by intervention group. Summaries, listings, datasets, or participant narratives may be provided, as appropriate, for those participants who die, who discontinue intervention due to an AE, or who experience a grade 3 or 4 AE or a serious AE. The proportion of participants with SAEs, the proportion of participants with grade 3 or 4 AEs, the proportion of participants with severe or life-threatening, bacterial, invasive fungal, viral or opportunistic infections (other than COVID-19), the incidence of grade 3 and 4 neutropenia and lymphocytopenia, and the incidence of increased ALT ≥3×ULN combined with increased bilirubin >2×ULN will be analyzed using a logistic regression model.

### Clinical Laboratory Tests

Laboratory data will be summarized by type of laboratory test. The laboratory abnormalities will be determined per the criteria specified and in accordance with the normal ranges of the clinical laboratory if no gradings are available. Descriptive statistics will be calculated for all laboratory analyte at baseline and for observed values and changes from baseline at each scheduled time point. A listing of participants with any laboratory results outside the reference ranges will be provided. A listing of participants with any markedly abnormal laboratory results will also be provided.

### Electrocardiogram

Electrocardiogram data will be summarized by ECG parameter. Descriptive statistics will be calculated at baseline and for observed values and changes from baseline at each scheduled time point. Frequency tabulations of the abnormalities will be made.

### Vital Signs

Vital signs including temperature, pulse/heart rate, respiratory rate, and blood pressure (systolic and diastolic) will be summarized over time, using descriptive statistics and/or graphically. The percentage of participants with values beyond clinically important limits will be summarized.

### Physical Examinations

Descriptive statistics of changes from baseline will be summarized at each scheduled time point. Physical examination findings will be summarized at each scheduled time point. Descriptive statistics will be calculated at baseline and for observed values and changes from baseline at each scheduled time point. Frequency tabulations of the abnormalities will be made.

### Other Analyses

### Pharmacokinetic Analyses

Serum sirukumab concentrations will be summarized using descriptive statistics. The concentrations below the lowest quantifiable sample concentration of the assay will be treated as zero in the summary statistics. All concentrations below the lowest quantifiable sample concentration of the assay or missing data will be labeled as such in the concentration data listing or statistical analysis dataset.

If feasible, population PK analysis of serum concentration-time data of sirukumab may be performed using nonlinear mixed-effects modeling. Data may be combined with other selected studies to support a relevant structural model. Available baseline participant characteristics (e.g., demographics, laboratory variables, etc.) may be tested as potential covariates affecting PK parameters. The results of the population PK analysis will be presented in a separate report.

### Biomarkers Analyses

Analysis of the relationship between various blood biomarkers, such as cytokines, and viral parameters, immunogenicity, safety and clinical outcome may be conducted. Descriptive statistics for actual values and (relative) changes from baseline for the different blood biomarkers assessed, will be tabulated for each biomarker at each applicable time point specified in the SoA. Statistics include n, mean, SD, CV, geometric mean, median, minimum, and maximum.

Statistical approaches to explore correlations between clinical outcome and blood biomarkers vary and depend on the different data types of the applied technology platforms, as well as on the extent of observed differences between participants. Analyses may be conducted and reported separately from this study.

### Immunogenicity Analyses

The incidence of antibodies to sirukumab will be summarized for all participants in the ITTi population with appropriate samples for detection of antibodies to sirukumab (i.e., participants with at least predose and the Day 28 sample obtained).

### Pharmacodynamic Analyses

Associations between baseline levels of biomarkers, immunogenicity tests, PK parameters and clinical response (primary endpoint and a selection of secondary endpoints) will be explored. More details will be provided in the statistical analysis plan.

### Pharmacogenomic Analyses

DNA samples will be analyzed if it is hypothesized that this may help resolve issues with the clinical data. DNA samples will be used for research related to sirukumab or COVID-19. Pharmacogenomic research may consist of the analysis of one or more candidate genes, of the analysis of genetic markers throughout the genome, or the analysis of the entire genome (as appropriate) to evaluate potential genetic associations with prognosis of clinical outcomes in patients and prediction of responsiveness to active treatment.

TABLE 14
SoA - Schedule of Activities

| | | | | Post Day 28 |
|---|---|---|---|---|
| | | | | Follow-up |
| Screening^{a} | Phase Day 1 to Day 28 | | | Phone Calls |
| Day | | | | |
| | | Day 2 to Day 28^{e} | | |
| | | or day of discharge | | |
| | | from hospital, | | |
| | | or study | Day 28 Visit if | |
| | | discontinuation, | discharged earlier | |
| Screening | Baseline^{a} | whichever comes | (Phone Call) Day | Day 56, Day 84, |
| Day-1 | Day 1 | first | 28 ± 3 days | Day 112 ± 7 days |
| Week | | | | |
| Week 1 | Week 1 | Week 1 to Week 4 | Week 4 | Week 8, 12, 16 |

General Screening/Baseline assessments

| | | | |
|---|---|---|---|
| ICF¹ | X | | |
| Inclusion/ | X | X^{g} | |
| exclusion criteria | | | |
| Demographics, | X | | |
| medical history^{h} | | | |
| Pregnancy | X^{b} | | Day 28 or at day |
| assessmentⁱ | | | of discharge |
| Targeted | X | | Any clinically |
| physical exam^{j} | | | significant |
| | | | findings to |
| | | | be reported |

Treatment administration

| | |
|---|---|
| Randomization^{d} | X |
| Administration | X |
| of study | |
| intervention^{d} | |

Concomitant medication

| | | | | | |
|---|---|---|---|---|---|
| Concomitant | X | X | Continuous | X | X |
| medication | | | | | |
| recording | | | | | |

Respiratory function related assessments

| Type of | X | X | Once per day | | |
|---|---|---|---|---|---|
| supplemental | | | | | |
| oxygen | | | | | |
| Resting SpO₂, | X | X | Minimum 4 | | |
| FiO₂ (if any)^{k} | | | times per day | | |
| Arterial pH, PaO₂, | As available | | | | |
| PaCO₂, SaO₂ | | | | | |
| Level of | X | X | Once per day | | |
| consciousness | | | while in the | | |
| (Glasgow coma | | | ICU | | |
| scale score)^{l} | | | | | |
| Pulmonary X-ray | X^{b} | X^{c} | As per SOC, | | |
| (or CT imaging if | | | report upon | | |
| X-ray not | | | worsening and | | |
| available) | | | last available | | |
| | | | | | Post Day 28 |
| | | | | | Follow-up |
| | Screening^{a} | Phase Day 1 to Day 28 | | | Phone Calls |
| | Day | | | | |
| | | | Day 2 to Day 28^{e} | | |
| | | | or day of discharge | | |
| | | | from hospital, | | |
| | | | or study | Day 28 Visit if | |
| | | | discontinuation, | discharged earlier | |
| | Screening | Baseline^{a} | whichever comes | (phone call) Day | Day 56, Day 84 |
| | Day-1 | Day 1 | first | 28 ± 3 days | Day 112 ± 7 days |
| | Week | | | | |
| | | | Week 1 (Day 2) | | |
| | Week 1 | Week 1 | to Week 4 | Week 4 | Week 8, 12, 16 |

General safety related assessments

| Standard 12-lead | X | X | Once per day | | |
|---|---|---|---|---|---|
| ECG | | | while in the | | |
| | | | ICU, then | | |
| | | | upon clinical | | |
| | | | indication | | |
| Hematology^{m}, | X^{b} | X | Preferred | | |
| chemistry^{m}, | | | daily if per | | |
| coagulation^{m} | | | local SOC, | | |
| | | | otherwise as | | |
| | | | a minimum on | | |
| | | | Day 3, Day 5, | | |
| | | | Day 7, Day 10, | | |
| | | | Day 14, Day 21, | | |
| | | | Day 28 | | |
| Vital signs (body | X | X | As per standard | | |
| temperature, | | | of care, minimum | | |
| pulse, SBP/DBP, | | | 4 times per day | | |
| respiratory rate) | | | while in the | | |
| | | | ICU, afterwards | | |
| | | | once per day | | |
| Bacterial, fungal, | | As per local SOC, | | | |
| viral infection | | any findings to be | | | |
| testing (blood, | | reported as AESI | | | |
| other)ⁿ | | | | | |
| Any AE^{o} | X | X | Continuous | X | X |
| Record vital | | | | X | X |
| status^{p} | | | | | |
| Record discharge | | | As applicable | | |
| from ICU, | | | | | |
| discharge | | | | | |
| from hospital | | | | | |
| Record | | | | X | X |
| readmission (s) | | | | | |
| post discharge | | | | | |

Virology Assessments

| Nasopharyngeal | X^{s, t} | X^{q} | Day 3, Day 5, | | X |
|---|---|---|---|---|---|
| swab for | | | Day 7, Day 10, | | |
| infections | | | Day 14, Day 21, | | |
| testing (PCR | | | Day 28 Optional: | | |
| quantification of | | | Day 4, Day 8 | | |
| SARS-CoV-2 and | | | | | |
| multiplex PCR for | | | | | |
| detection of co- | | | | | |
| infections)^{r, t} | | | | | |
| If participants | | | | | |
| are intubated, an | | | | | |
| endotracheal | | | | | |
| sample for | | | | | |
| quantification of | | | | | |
| SARS-CoV-2 is to | | | | | |
| be taken in | | | | | |
| addition to the | | | | | |
| NP swab^{u} | | | | | |
| Stool sample | X | X | Day 3, Day 5, | | X |
| (optional) | | | Day 7, Day 10, | | |
| | | | Day 14, Day 21, | | |
| | | | Day 28 Optional: | | |
| | | | Day 4, Day 8 | | |
| Blood sample | | X | Day 7, Day 14, | | |
| for SARS-CoV- | | | Day 21, Day 28 | | |
| 2 viremia | | | | | |
| | | | | | Post Day 28 |
| | | | | | Follow-up |
| | Screening^{a} | Phase Day 1 to Day 28 | | | Phone Calls |
| | Day | | | | |
| | | | Day 2 to Day 28^{e} | | |
| | | | or day of discharge | | |
| | | | from hospital, | | |
| | | | or study | Day 28 Visit if | |
| | | | discontinuation, | discharged earlier | |
| | Screening | Baseline^{a} | whichever comes | (phone call) Day | Day 56, Day 84 |
| | Day-1 | Day 1 | first | 28 ± 3 days | Day 112 ± 7 days |
| | Week | | | | |
| | | | Week 1 to | | |
| | Week 1 | Week 1 | Week 4 | Week 4 | Week 8, 12, 16 |

Biomarker/Pharmacology Assessments

| | | | |
|---|---|---|---|
| Blood samples | X | X | Day 5, Day 14, |
| for biomarker | | | Day 21, Day 28^{x} |
| research^{v} | | | Optional: Day 3, |
| | | | Day 4, Day 7, |
| | | | Day 8, Day 10 |
| Blood samples | | X | Day 14, Day 21, |
| for pharmacology^{w} | | | Day 28^{x} |
| Blood samples | | X | Day 5, Day 14, |
| for cellular | | | Day 21, Day 28 |
| profiling | | | |

Exploratory Biomarkers/Pharmacogenomics

| | | |
|---|---|---|
| PAXgene blood for | X | Day 5, Day 14, |
| RNA profilingy | | Day 21, Day 28 |
| Whole blood for | X^{aa} | |
| DNA profiling | | |
| (optional)^{z} | | |

Note:
If multiple assessments are scheduled for the same timepoint, it is recommended that procedures be performed in the following sequence: ECG, oxygen saturation, vital signs, blood sampling. Baseline tests must be completed prior to study intervention administration.
Abbreviations: ACVPU: alert, confusion, voice, pain, unresponsive; DBP: diastolic blood pressure; ECG: electrocardiogram; AE: adverse event, AESI: adverse event of special interest, ICF: informed consent form; ICU: intensive care unit; PCR: polymerase chain reaction; SAE: serious adverse event; SpO₂: peripheral capillary oxygen saturation, SBP: systolic blood pressure, PaO₂: partial pressures of oxygen in arterial blood, PaCO₂: partial pressure of carbon dioxide in arterial blood, SaO₂: arterial oxygen saturation
Footnotes
^{a}All screening and baseline assessments may take place on the same day. In such case, the tests do not need to be repeated. All screening assessments should take place prior to randomization and all baseline assessments prior to study intervention administration.
^{b}The assessment schedule will remain the same for participants still hospitalized after Day 28.
^{c}Consent to be obtained before the first study-related activity. An exception is in the case of an emergency enrollment in which the informed consent can be obtained as soon as possible.
^{d}Please note that one of the inclusion criteria is “Requiring supplemental oxygen delivered at a flow ≥6 L/min, to sustain a SpO2 >93% regardless of device/route used (corresponds to category 3 or 4 on the 6-point ordinal scale)”. For these patients, confirm, if safe to do so, that the participant has an SpO2 ≤ 93% on 4 L/min of supplemental oxygen, which constitutes a requirement or a need for at least 6 L/min.
^{e}If a participant's clinical status changes after screening but before the study intervention is given such that he or she no longer meets all eligibility criteria, then the participant should be excluded from participation in the study.
^{f}Medical history should include collecting onset of COVID-19 symptoms, prior therapy, and date of SARS-CoV-2 diagnosis if available.
^{g}A pregnancy assessment is to be performed in women of childbearing potential only.
^{h}Results from the blood chemistry, hematology, and coagulation test, pregnancy test, and pulmonary X-ray, completed as SOC, taken up to 2 days prior to screening will be accepted as screening assessments.
ⁱTargeted physical examination includes lung auscultation and any examination as indicated by the patient's medical history. Height and body weight are only to be measured at screening if not already available in the participant's chart and if practically feasible.
^{j}Participants need to receive study intervention preferably within 4 hours but no later than 6 hours after randomization.
^{k}Supplemental oxygen/percentage of inspired oxygen (FiO2) use (if any) will be measured (simultaneously with SpO2, and at any time of blood gas measurements) to monitor the patient's status regarding gas exchange as applicable. The following will be recorded:
- Oxygen delivery device (eg, nasal cannula, simple face mask, nonrebreather mask, high flow nasal cannula, non-invasive ventilation, invasive mechanical ventilation, extracorporeal life support, etc).
- Oxygen flow rate in liters/min.
- Record FiO2 and SpO2 data 4 times per day, and at any time of blood gas measurements.
Record values that are sustained for at least 1 hour. For the analyses, the worst values sustained for at least 1 hour, on the highest level of oxygen supplementation method of the day, will be used
- If a patient is using more than one device (e.g., extracorporeal life support and invasive ventilation), information (the worst recording) from both devices will be recorded separately.
- If a patient does not need oxygen supplement, this should also be recorded.
^{l}The final worst score of the Glasgow Coma Scale of the day needs to be recorded in the eCRF. The level of sedation of the participant will be derived from the type of medication entered for indication sedation on the Concomitant Medication page of the eCRF.
^{m}Laboratory testing will be performed, this may include:
- Blood chemistry: total protein, albumin, aspartate aminotransferase (AST), alanine aminotransferase (ALT), alkaline phosphatase (ALP), total bilirubin, total cholesterol, low-density lipoprotein (LDL), high-density lipoprotein (HDL), triglycerides, glucose, sodium, potassium, calcium, phosphate, magnesium, chloride, bicarbonate, gammaglutamyltransferase (GGT), creatinine, blood urea nitrogen (BUN), uric acid, creatine phosphokinase (CPK), lactate dehydrogenase (LDH), lactate, C-reactive protein (CRP), ferritin, D-dimer, pro-calcitonin, and troponin.
- Hematology: hemoglobin, hematocrit, platelet count, red blood cell (RBC) count, RBC indices (mean corpuscular volume [MCV], mean corpuscular hemoglobin [MCH], % reticulocytes), white blood cell (WBC) count with differential (neutrophils, lymphocytes, monocytes, basophils, eosinophils).
- Coagulation tests: prothrombin time (PT), activated partial thromboplastin time (aPTT), and fibrinogen.
Hematology and liver parameters should be reported to the sponsor via eCRF data entry within approximately 72 hours from results becoming available.
ⁿCulture results (bacterial, fungal, or viral) including site of infection and specimen source (bronchoalveolar lavage [BAL], tracheal aspirate, sputum, blood, urine, etc.), performed as part of patients' workup for new infections, should be reported. Analyses will be performed by the local laboratory.
^{o}For participants discharged or who discontinued the study prior to Day 28 and did not withdraw consent, a phone call will be conducted on Day 28 and during the post Day 28 Phase to assess the vital status, the occurrence of AEs, and the history of readmission since last contact.
^{p}Whenever possible, vital status will be recorded if the patient is alive. If the participant is deceased, date and cause of mortality should be recorded in the eCRF. Death should be documented as SAE.
^{q}NP swabs will be used to collect secretions from participants to explore quantification of viral load of SARS-CoV-2. For each participant, NP sampling should be done at approximately the same time (±4 hours) on each sampling day and from the same nostril.
^{r}If an NP sample for detection of SARS-CoV-2 (local SOC) will be collected on the same day as the NP sample for quantification of SARS-CoV-2 (central lab), only one NP sample should be collected. The sample should be aliquoted and the remaining aliquots of the NP samples should be stored and sent to the central lab for quantification of SARS-CoV-2.
^{s}SARS-CoV-2 positivity should be documented based on local testing on any specimen, by RT-PCR or any other commercial or public health assay, any time before randomization. This might require a local test using a NP swab obtained at screening.
^{t}After randomization, SARS-CoV-2 positivity will be confirmed in a central lab by quantitative RT-PCR. The baseline sample needs to be collected predose, as close as possible to dosing.
^{u}If the participant is intubated, endotracheal samples need to be taken at the same time as the NP swab. If taking both NP and endotracheal samples is not feasible, the NP sample should be given priority.
^{v}Lab testing for detection of SARS-CoV-2 on the NP swab at day of hospital discharge. If SARS CoV-2 positive, an additional NP swab will be taken every 7 days, if feasible for the site and tested, until SARS-CoV-2 negative
^{w}For participants discharged after Day 28 an additional sample should be taken on the day of discharge.
^{x}If viral RNA is detected in NP samples at day of discharge, all possible efforts will be made to follow-up participants and collect samples until viral RNA is negative, considering the current pandemic and related logistical challenges.
^{y}Includes plasma and serum samples for exploratory biomarker endpoint evaluation (including but not limited to IL-6, pro-calcitonin, CRP, ferritin, LDH and D-dimer serum concentrations) and SARS-CoV-2 specific antibodies (IgM, IgG).
^{z}For participants discharged after Day 28 an additional sample should be taken on the day of discharge.
^{aa}Includes serum samples for measurement of PK, antibodies to sirukumab, and IL-6. On Day 1, a predose and a postdose (within 30 minutes after the end of infusion) sample should be collected. The postdose sample should be collected from the arm contralateral to that used for IV infusion. On Day 1 (predose) and Day 28 the PK, antibodies to sirukumab, and IL-6 will be evaluated. On Day 1 (postdose), Day 14, and Day 21 the PK and IL-6 will be evaluated.
^{bb} PAXgene RNA tubes should always be used last for any blood draw.
^{cc} An optional pharmacogenetics blood sample (DNA) will be obtained from those participants who gave consent (where local regulations permit).
^{dd} Sample can be also taken at any other time point after study drug intervention on Day 1.

TABLE 11
Protocol-Required Safety Laboratory Assessments

| Laboratory | | | |
|---|---|---|---|
| Assessments | Parameters | | |
| Hematology | Platelet count | RBC Indices: | White Blood Cell |
| Testing done locally | Red blood cell count | MCV | (WBC) count with |
| | Hemoglobin | MCH | Differential: |
| | Hematocrit | % Reticulocytes | Neutrophils |
| | | | Lymphocytes |
| | | | Monocytes |
| | | | Eosinophils |
| | | | Basophils |

| | Note: A WBC evaluation may include any abnormal cells, which will then be | |
|---|---|---|
| | reported by the laboratory. A RBC evaluation may include abnormalities in the | |
| | RBC count, RBC parameters, or RBC morphology, which will then be reported by | |
| | the laboratory. In addition, any other abnormal cells in a blood smear will | |
| | also be reported. | |
| Clinical | Sodium | Creatine phosphokinase (CPK) |
| Chemistry | Potassium | Lactic acid dehydrogenase (LDH) |
| Testing done locally | Chloride | Lactate |
| | Blood urea nitrogen (BUN) | Uric acid |
| | Creatinine | Calcium |
| | Glucose | Phosphate |
| | Aspartate aminotransferase (AST)/ | Albumin |
| | Serum glutamic-oxaloacetic | Total protein |
| | Alanine aminotransferase (ALT)/ | Total cholesterol |
| | Serum glutamic-oxaloacetic | LDL |
| | Gamma-glutamyltransferase (GGT) | HDL |
| | Carbon dioxide | Triglycerides |
| | Total bilirubin | Magnesium |
| | Alkaline phosphatase | Troponin I and T |
| | | Pro-calcitonin |
| | | CRP |
| | | Ferritin |
| | Note: Hematology and liver parameters should be reported to the sponsor | |
| | via eCRF data entry approximately 72 hours from results becoming available. | |
| Coagulation tests | Prothrombin time, partial prothrombin time, fibrinogen | |
| Testing done locally | | |
| Arterial blood gases | pH, PaO₂, PaCO₂, SaO₂ (as per local SOC) | |
| Testing done locally | | |
| Other Tests | Testing done locally | Testing done centrally by |
| | | the sponsor |
| | Serum pregnancy testing (or | NP swabs for infections testing |
| | urine pregnancy testing in case | (PCR quantification of SARS-CoV-2 |
| | of emergency situations) for all | and multiple PCR for detection |
| | women of childbearing potential | of co-infections) |
| | (at screening) | Endotracheal aspirate (only if |
| | Bacterial, fungal, or viral | intubated) for central virology |
| | infection testing (as per local | testing (PCR SARS-CoV-2) |
| | SOC) | Blood samples for cellular profiling |
| | NP swabs for virology testing | Blood samples for RNA profiling |
| | (PCR SARS-CoV-2). | Whole blood for DNA profiling |
| | Endotracheal aspirate (only | (optional) |
| | if intubated) for virology | Pharmacology samples (including |
| | testing (PCR SARS-CoV-2) | testing for IL-6 and antibodies |
| | | to sirukumab) |

The invention can be described with reference to the following numbered embodiments:
1. Sirukumab for use in the treatment or prevention of infection of a human with SARS-CoV-1 and/or SARS-CoV-2.
2. Sirukumab, according to embodiment 1, for use in the treatment of infection of a human with SARS-CoV-1 and/or SARS-CoV-2.
3. Sirukumab, according to embodiment 1, for use in the prevention of infection of a human with SARS-CoV-1 and/or SARS-CoV-2.
4. Sirukumab, for use according to any one of the preceding embodiments, wherein the infection is a SARS-CoV-2 infection.
5. Sirukumab, for use according to embodiment 4, wherein the infection with SARS-CoV-2 is COVID-19.
6. Sirukumab, for use according to any one of the preceding embodiments, wherein the sirukumab is administered intravenously, orally or by pulmonary delivery.
7. Sirukumab, for use according to embodiment 6, wherein the sirukumab is administered intravenously.
8. Sirukumab, for use according to any one of the embodiments 6 and 7, wherein the sirukumab is administered once, twice or three times per day.
9. Sirukumab, for use according to any one of embodiments 6 or 8Error! Reference source not found, wherein the sirukumab is administered at a dose of 3 to 10 mg per kg weight of the patient, preferably 4 to 8 mg per kg weight of the patient, more preferably 5 mg per kg weight of the patient.
10. Sirukumab, for use according to any one of embodiments 6 to 9, wherein the sirukumab is administered as a pharmaceutically acceptable aqueous solution comprising 50 to 150 mg/mL sirukumab, preferably 100 mg/mL sirukumab.
11. Sirukumab, for use according to embodiments 10, wherein the acceptable aqueous solution further comprises 30 to 50 mg/mL sorbitol, 0.2 to 0.6 glacial Acetic Acid mg/mL, 0.5 to 1 mg/mL sodium acetate, and 0.2 to 0.6 mg/mL polysorbate, preferably, the antibody formulation further comprises 43 mg/mL, 0.4 glacial Acetic Acid mg/mL, 0.7 mg/mL sodium acetate, and 0.4 mg/mL polysorbate.
12. Sirukumab, for use according to any one of embodiments 6 to 11, wherein the sirukumab is administered to the treated human in simultaneous or sequential combination with one or more other antiviral agents.
13. Sirukumab, for use according to embodiment 12, wherein the one or more antiviral agents is independently selected from the group consisting of small molecules, vaccines, proteins, plasma derived agents.
14. Sirukumab, for use according to any one of embodiments 12 or 13, wherein the one or more antiviral agents comprises a small molecule.
15. Sirukumab, for use according to any one of embodiments 13 to 14, wherein the small molecule(s) is/are independently selected from the group consisting of favipiravir, remdesivir, ifenprodil, chloroquine, umifenovir, APN01, galidesivir, ritonavir, BPI 002, OYA1 and SNG001.
16. Sirukumab, for use according to any one of the preceding embodimentsError! Reference source not found, wherein the sirukumab comprises a heavy chain variable region amino acid sequence of SEQ ID NO:99 and a light chain variable region of SEQ ID NO:97.
17. Sirukumab, for use according to any one of the preceding embodimentsError! Reference source not found, wherein the treated human achieves improvement of at least one category relative to reference on the 6-point clinical recovery scale of clinical status for at least 48 hours and up to 28 days selected from the group consisting of: not hospitalized, hospitalization with the patient not requiring supplemental oxygen, hospitalization with non-invasive ventilation or high flow oxygen device, hospitalization with invasive mechanical ventilation or extracorporeal membrane oxygenation (ECMO) and death.
18. Sirukumab, for use according to embodiment 17Error! Reference source not found, wherein the treated human achieves improvement of at least two categories relative to reference on the 6-point clinical recovery scale of clinical status for 28 days.
19. Use of Sirukumab in the manufacture of a medicament for the treatment or prevention of infection of a human with SARS-CoV-1 and/or SARS-CoV-2.
20. The use according to embodiment 19, wherein the medicament is for the treatment of infection of a human with SARS-CoV-1 and/or SARS-CoV-2.
21. The use according to embodiment 19, wherein the medicament is for the prevention of infection of a human with SARS-CoV-1 and/or SARS-CoV-2.
22. The use according to any one of embodiments 19 to 21, wherein the infection with SARS-CoV-2 infection.
23. The use according to embodiment 22, wherein the infection with SARS-CoV-2 is COVID-19.
24. The use according to any one of embodiments 19 to 23, wherein the sirukumab is administered intravenously, orally or by pulmonary delivery.
25. The use according to embodiment 24, wherein the sirukumab is administered intravenously.
26. The use according to any one of embodiments 24 and 25, wherein the sirukumab is administered once, twice or three times per day.
27. The use according to any one of embodiments 24 to 26Error! Reference source not found, wherein the sirukumab is administered at a dose of 3 to 10 mg per kg weight of the patient, preferably 4 to 8 mg per kg weight of the patient, more preferably 5 mg per kg weight of the patient.
28. The use according to any one of embodiments 24 to 27, wherein the sirukumab is administered as a pharmaceutically acceptable aqueous solution comprising 50 to 150 mg/mL sirukumab, preferably 100 mg/mL sirukumab.
29. The use according to embodiments 28, wherein the acceptable aqueous solution further comprises 30 to 50 mg/mL sorbitol, 0.2 to 0.6 glacial Acetic Acid mg/mL, 0.5 to 1 mg/mL sodium acetate, and 0.2 to 0.6 mg/mL polysorbate, preferably, the antibody formulation further comprises 43 mg/mL sorbitol, 0.4 glacial Acetic Acid mg/mL, 0.7 mg/mL sodium acetate, and 0.4 mg/mL polysorbate.
30. The use according to any one of embodiments 24 to 29, wherein the sirukumab is administered to the treated human in simultaneous or sequential combination with one or more other antiviral agents.
31. The use according to embodiment 30, wherein the one or more antiviral agents is independently selected from the group consisting of small molecules, vaccines, proteins, plasma derived agents.
32. The use according to any one of embodiments 30 and 31, wherein the one or more antiviral agents comprises a small molecule.
33. The use according any one of embodiments 31 and 32, wherein the small molecule(s) is/are independently selected from the group consisting of favipiravir, remdesivir, ifenprodil, chloroquine, umifenovir, APN01, galidesivir, ritonavir, BPI 002, OYA1 and SNG001.
34. The use according to any one of the embodiments 19 to 33Error! Reference source not found, wherein the sirukumab comprises a heavy chain variable region amino acid sequence of SEQ ID NO:99 and a light chain variable region of SEQ ID NO:97.
35. The use according to any one of the preceding embodiments Error! Reference source not found, wherein the treated human achieves improvement of at least one category relative to reference on the 6-point clinical recovery scale of clinical status selected from the group consisting of: not hospitalized, hospitalization with the patient not requiring supplemental oxygen, hospitalization with non-invasive ventilation or high flow oxygen device, hospitalization with invasive mechanical ventilation or extracorporeal membrane oxygenation (ECMO) and death.
36. The use according to embodiment 16 Error! Reference source not found, wherein the treated human achieves improvement of the at least one category for at least 48 hours and up to 28 days.

### Definitions

The term “comprising” encompasses “including” as well as “consisting” e.g., a composition “comprising” X may consist exclusively of X or may include something additional e.g. X+Y.

The word “substantially” does not exclude “completely” e.g., a composition which is “substantially free” from Y may be completely free from Y. Where necessary, the word “substantially” may be omitted from the definition of the invention.

The term “about” in relation to a numerical value x means, for example, x±10%. In one embodiment, the word “about” may be omitted.

All references cited herein are incorporated by reference in their entirety.

1. A method for treating an infection of SARS-CoV-1 and/or SARS-CoV-2 in a human patient comprising administering to the patient an effective amount of a pharmaceutical composition comprising an anti-IL6 agent.

2. The method of claim 1, wherein the anti-IL6 agent is an anti-IL6 antibody that blocks binding of IL-6 to IL-6 receptor.

3. The method of claim 2, wherein the patient is a COVID-19 patient with severe hypoxemia at risk for Acute Respiratory Distress Syndrome (ARDS).

4. The method of claim 2, wherein the patient is a COVID-19 patient with ARDS onset.

5. The method of claim 2 or 4, wherein the antibody comprises a heavy chain variable region and a light chain variable region comprising:
i) a CDRH1 amino acid sequence of SEQ ID NO: 135;
ii) a CDRH2 amino acid sequence of SEQ ID NO: 136;
iii) a CDRH3 amino acid sequence of SEQ ID NO: 137;
iv) a CDRL1 amino acid sequence of SEQ ID NO: 132;
v) a CDRL2 amino acid sequence of SEQ ID NO: 133; and
vi) a CDRL3 amino acid sequence of SEQ ID NO: 134; and wherein X₁ is A or G, X₂ is S or R, X₃ is H, I, S, or Y, X₄ is S or Y, X₅ is S or F, X₆ is F, L, M, or T, X₇ is N or E, X₈ is A or T, X₉ is M, C, S or Q, X₁₀ is Q or C, X₁₁ is T or Q, X₁₂ is F, S, or T, X₁₃ is S or P, X₁₄ is L or M, X₁₅ is A or I, X₁₆ is S or P, X₁₇ is Y or W, X₁₈ is T, E, or Y, X₁₉ is Y or F, X₂₀ is P, S, D, or Y, X₂₁ is V or D, X₂₂ is T or A, X₂₃ is G or P, X₂₄ is S, Y, T, or N, and X₂₅ is Y, T, F, or I.

6. The method of claim 5, wherein the antibody comprises a heavy chain variable region and a light chain variable region comprising:
i) a CDRH1 amino acid sequence of SEQ ID NO: 39;
ii) a CDRH2 amino acid sequence of SEQ ID NO: 59;
iii) a CDRH3 amino acid sequence of SEQ ID NO: 89;
iv) a CDRL1 amino acid sequence of SEQ ID NO: 3;
v) a CDRL2 amino acid sequence of SEQ ID NO: 21; and
vi) a CDRL3 amino acid sequence of SEQ ID NO: 29.

7. The method of claim 6 wherein the antibody comprises a heavy chain variable region amino acid sequence of SEQ ID NO:99 and a light chain variable region of SEQ ID NO:97.

8. The method of claim 7, wherein the antibody is sirukumab (CNTO136).

9. The method of claim 8, wherein the antibody is administered intravenously at a dose of about 1-10 mg of antibody per kg weight of the patient.

10. The method of claim 9, wherein the antibody is administered intravenously at a dose of about 5 mg of antibody per kg weight of the patient.

11. The method of claim 10, wherein the antibody is in an aqueous solution of 100 mg/ml antibody, Sorbitol 43 mg/mL, Glacial Acetic Acid 0.4 mg/mL, Sodium Acetate 0.7 mg/mL, Polysorbate 0.4 mg/mL.

12. The method of claim 2 or 4, wherein the antibody comprises a heavy chain variable region amino acid sequence of SEQ ID NO:139 and a light chain variable region amino acid sequence of SEQ ID NO:140.

13. The method of claim 2 or 4, wherein the antibody comprises a CDRH1 amino acid sequence of SEQ ID NO:141, a CDRH2 amino acid sequence of SEQ ID NO:142, a CDRH3 amino acid sequence of SEQ ID NO:143, a CDRL1 amino acid sequence of SEQ ID NO:144, a CDRL2 amino acid sequence of SEQ ID NO:145, a CDRL3 amino acid sequence of SEQ ID NO:146.

14. The method of claim 8, wherein the patient achieves improvement of at least one category relative to reference on a 6-point clinical recovery scale of clinical status for at least 48 hours and up to 28 days selected from the group consisting of: not hospitalized, hospitalization with the patient not requiring supplemental oxygen, hospitalization with non-invasive ventilation or high flow oxygen device, hospitalization with invasive mechanical ventilation or extracorporeal membrane oxygenation (ECMO) and death.

15. The method of claim any of claim 14, wherein the patient achieves improvement of at least two categories relative to reference on the 6-point clinical recovery scale of clinical status after 28 days.

16. The method of claim 8, wherein the antibody is administered to the patient in simultaneous or sequential combination with one or more antiviral agents.

17. The method of claim 16, wherein the one or more antiviral agents is independently selected from the group consisting of small molecules, vaccines, proteins, plasma derived agents.

18. The method of claim 17, wherein the one or more antiviral agents comprises a small molecule.

19. The method of claim 18, wherein the small molecule(s) is/are independently selected from the group consisting of favipiravir, remdesivir, ifenprodil, chloroquine, umifenovir, APN01, galidesivir, ritonavir, BPI 002, OYA1 and SNG001.

20. The method of claim 16, wherein the patient has previously been administered an antiviral agent but the antiviral agent has failed to treat or infection with SARS-CoV-1 and/or SARS-CoV-2.

21. An isolated anti-IL6 antibody comprising: (i) a heavy chain variable region amino acid sequence of SEQ ID NO:99 and a light chain variable region amino acid sequence of SEQ ID NO:97; (ii) a CDRH1 a CDRH1 amino acid sequence SEQ ID NO: 39, a CDRH2 amino acid sequence of SEQ ID NO: 59, a CDRH3 amino acid sequence of SEQ ID NO: 89, a CDRL1 amino acid sequence of SEQ ID NO: 3, a CDRL2 amino acid sequence of SEQ ID NO: 21 and a CDRL3 amino acid sequence of SEQ ID NO: 29; or (iii) a CDRH1 amino acid sequence of SEQ ID NO: 39, a CDRH2 amino acid sequence of SEQ ID NO: 59, a CDRH3 amino acid sequence of SEQ ID NO: 89, a CDRL1 amino acid sequence of SEQ ID NO: 3, a CDRL2 amino acid sequence of SEQ ID NO: 21 and a CDRL3 amino acid sequence of SEQ ID NO: 29, for use in the treatment of infection of a human with SARS-CoV-1 and/or SARS-CoV-2.

22. An isolated anti-IL6 antibody for use according to claim 21, wherein the infection is a SARS-CoV-2 infection.

23. An isolated anti-IL6 antibody for use according to claim 22, wherein the infection with SARS-CoV-2 is COVID-19.

24. An isolated anti-IL6 antibody for use according to any of claims 21-23, wherein the patient achieves improvement of at least one category relative to reference on the 6-point clinical recovery scale of clinical status for at least 48 hours and up to 28 days selected from the group consisting of: not hospitalized, hospitalization with the patient not requiring supplemental oxygen, hospitalization with non-invasive ventilation or high flow oxygen device, hospitalization with invasive mechanical ventilation or extracorporeal membrane oxygenation (ECMO) and death.

25. An isolated anti-IL6 antibody for use according to claim 23, wherein the patient achieves improvement of at least two categories relative to reference on the 6-point clinical recovery scale of clinical status after 28 days.

26. An isolated anti-IL6 antibody for use according to claim 21, wherein the antibody is sirukumab (CNTO136).

27. An isolated anti-IL6 antibody for use according to claim 26, wherein the antibody is administered intravenously at a dose of about 1-10 mg of antibody per kg weight of the patient.

28. An isolated anti-IL6 antibody for use according to claim 27, wherein the antibody is administered intravenously at a dose of about 5 mg of antibody per kg weight of the patient.

29. An isolated anti-IL6 antibody for use according to claim 28, wherein the antibody is in an aqueous solution of 100 mg/ml antibody, Sorbitol 43 mg/mL, Glacial Acetic Acid 0.4 mg/mL, Sodium Acetate 0.7 mg/mL, Polysorbate 0.4 mg/mL.

30. An isolated anti-IL6 antibody for use according to any of claim 29, wherein the antibody is administered to the human in simultaneous or sequential combination with one or more antiviral agents.

31. An isolated anti-IL6 antibody for use according to claim 30, wherein the one or more antiviral agents is independently selected from the group consisting of small molecules, vaccines, proteins, plasma derived agents.

32. An isolated anti-IL6 antibody for use according to claim 31, wherein the one or more antiviral agents comprises a small molecule.

33. An isolated anti-IL6 antibody for use according to claim 32, wherein the small molecule(s) is/are independently selected from the group consisting of favipiravir, remdesivir, ifenprodil, chloroquine, umifenovir, APN01, galidesivir, ritonavir, BPI 002, OYA1 and SNG001.

34. An isolated anti-IL6 antibody for use according to claim 33, wherein the human has previously been administered an antiviral agent but the antiviral agent has failed to treat or infection with SARS-CoV-1 and/or SARS-CoV-2.

35. Products comprising an anti-IL6 antibody and an antiviral agent as listed in claim 30 as a combined preparation for simultaneous, separate or sequential use in the treatment of infection of a human with SARS-CoV-1 and/or SARS-CoV-2.


SEQUENCE LISTING (146 sequences)

SEQ ID NO: 1 - protein, 10 aa, Homo sapiens
SASHSVSYMY

SEQ ID NO: 2 - DNA, 30 bp, Homo sapiens
agtgccagccatagtgtaagttacatgtac

SEQ ID NO: 3 - protein, 10 aa, Homo sapiens
SASISVSYMY

SEQ ID NO: 4 - DNA, 30 bp, Homo sapiens
agtgccagcattagtgtaagttacatgtac

SEQ ID NO: 5 - protein, 10 aa, Homo sapiens
SARSSVSYMY

SEQ ID NO: 6 - DNA, 30 bp, Homo sapiens
agtgcccggtcaagtgtaagttacatgtac

SEQ ID NO: 7 - protein, 10 aa, Homo sapiens
SASYSVSYMY

SEQ ID NO: 8 - DNA, 30 bp, Homo sapiens
agtgccagctatagtgtaagttacatgtac

SEQ ID NO: 9 - protein, 10 aa, Homo sapiens
SASSSVFYMY

SEQ ID NO: 10 - DNA, 30 bp, Homo sapiens
agtgccagctcaagtgtattttacatgtac

SEQ ID NO: 11 - protein, 10 aa, Homo sapiens
SGSSYVSYMY

SEQ ID NO: 12 - DNA, 30 bp, Homo sapiens
agtggcagctcatatgtaagttacatgtac

SEQ ID NO: 13 - protein, 10 aa, Homo sapiens
SALSSVSYMY

SEQ ID NO: 14 - DNA, 30 bp, Homo sapiens
agtgccctgtcaagtgtaagttacatgtac

SEQ ID NO: 15 - protein, 10 aa, Homo sapiens
SASSSVSYMY

SEQ ID NO: 16 - DNA, 30 bp, Homo sapiens
agtgccagctcaagtgtaagttacatgtac

SEQ ID NO: 17 - protein, 7 aa, Homo sapiens
DFSNLAS

SEQ ID NO: 18 - DNA, 21 bp, Homo sapiens
gacttttccaacctggcttct

SEQ ID NO: 19 - protein, 7 aa, Homo sapiens
DLSNLAS

SEQ ID NO: 20 - DNA, 21 bp, Homo sapiens
gacctgtccaacctggcttct

SEQ ID NO: 21 - protein, 7 aa, Homo sapiens
DMSNLAS

SEQ ID NO: 22 - DNA, 21 bp, Homo sapiens
gacatgtccaacctggcttct

SEQ ID NO: 23 - protein, 7 aa, Homo sapiens
DTSNLTS

SEQ ID NO: 24 - DNA, 21 bp, Homo sapiens
gacacatccaacctgacgtct

SEQ ID NO: 25 - protein, 7 aa, Homo sapiens
DTSELAS

SEQ ID NO: 26 - DNA, 21 bp, Homo sapiens
gacacatccgagctggcttct

SEQ ID NO: 27 - protein, 7 aa, Homo sapiens
DTSNLAS

SEQ ID NO: 28 - DNA, 21 bp, Homo sapiens
gacacatccaacctggcttct

SEQ ID NO: 29 - protein, 9 aa, Homo sapiens
MQWSGYPYT

SEQ ID NO: 30 - DNA, 27 bp, Homo sapiens
atgcagtggagtggttacccatacacg

SEQ ID NO: 31 - protein, 9 aa, Homo sapiens
CQWSGYPYT

SEQ ID NO: 32 - DNA, 27 bp, Homo sapiens
tgtcagtggagtggttacccatacacg

SEQ ID NO: 33 - protein, 9 aa, Homo sapiens
SCWSGYPYT

SEQ ID NO: 34 - DNA, 27 bp, Homo sapiens
tctgtgtggagtggttacccatacacg

SEQ ID NO: 35 - protein, 9 aa, Homo sapiens
SQWSGYPYT

SEQ ID NO: 36 - DNA, 27 bp, Homo sapiens
tctcagtggagtggttacccatacacg

SEQ ID NO: 37 - protein, 10 aa, Homo sapiens
GFTFSSFALS

SEQ ID NO: 38 - DNA, 30 bp, Homo sapiens
ggattcacctttagtagctttgccctttct

SEQ ID NO: 39 - protein, 10 aa, Homo sapiens
GFTFSPFAMS

SEQ ID NO: 40 - DNA, 30 bp, Homo sapiens
ggattcacctttagtccttttgccatgtct

SEQ ID NO: 41 - protein, 10 aa, Homo sapiens
GFQFSSFAMS

SEQ ID NO: 42 - DNA, 30 bp, Homo sapiens
ggattccagtttagtagctttgccatgtct

SEQ ID NO: 43 - protein, 10 aa, Homo sapiens
GFTTSSFAMS

SEQ ID NO: 44 - DNA, 30 bp, Homo sapiens
ggattcaccactagtagctttgccatgtct

SEQ ID NO: 45 - protein, 10 aa, Homo sapiens
GFQFSPFAMS

SEQ ID NO: 46 - DNA, 30 bp, Homo sapiens
ggattccagtttagtccttttgccatgtct

SEQ ID NO: 47 - protein, 10 aa, Homo sapiens
GFTFSSFAMS

SEQ ID NO: 48 - DNA, 30 bp, Homo sapiens
ggattcacctttagtagctttgccatgtct

SEQ ID NO: 49 - protein, 17 aa, Homo sapiens
KASSGGSYTYYPDTVTG

SEQ ID NO: 50 - DNA, 51 bp, Homo sapiens
aaagcgagtagtggtgggagttacacctactatcctgacactgtgacgggc

SEQ ID NO: 51 - protein, 17 aa, Homo sapiens
KISSGGSYEYYPDTVTG

SEQ ID NO: 52 - DNA, 51 bp, Homo sapiens
aaaattagtagtggtgggagttacgagtactatcctgacactgtgacgggc

SEQ ID NO: 53 - protein, 17 aa, Homo sapiens
KISSGGSYYYYPDTVTG

SEQ ID NO: 54 - DNA, 51 bp, Homo sapiens
aaaattagtagtggtgggagttactattactatcctgacactgtgacgggc

SEQ ID NO: 55 - protein, 17 aa, Homo sapiens
KISSGGSWTYYPDTVTG

SEQ ID NO: 56 - DNA, 51 bp, Homo sapiens
aaaattagtagtggtgggagttggacctactatcctgacactgtgacgggc

SEQ ID NO: 57 - protein, 17 aa, Homo sapiens
KISPGGSYTYYPDTVTG

SEQ ID NO: 58 - DNA, 51 bp, Homo sapiens
aaaattagtccgggtgggagttacacctactatcctgacactgtgacgggc

SEQ ID NO: 59 - protein, 17 aa, Homo sapiens
KISPGGSWTYYSDTVTG

SEQ ID NO: 60 - DNA, 51 bp, Homo sapiens
aaaattagtccgggtgggagttggacctactattctgacactgtgacgggc

SEQ ID NO: 61 - protein, 17 aa, Homo sapiens
KISSGGSYTYYPDTVTG

SEQ ID NO: 62 - DNA, 51 bp, Homo sapiens
aaaattagtagtggtgggagttacacctactatcctgacactgtgacgggc

SEQ ID NO: 63 - protein, 17 aa, Homo sapiens
KISSGGSYTYFPDTVTG

SEQ ID NO: 64 - DNA, 51 bp, Homo sapiens
aaaattagtagtggtgggagttacacctactttcctgacactgtgacgggc

SEQ ID NO: 65 - protein, 17 aa, Homo sapiens
KISSGGSYTYYPDTVAG

SEQ ID NO: 66 - DNA, 51 bp, Homo sapiens
aaaattagtagtggtgggagttacacctactatcctgacactgtggctggc

SEQ ID NO: 67 - protein, 17 aa, Homo sapiens
KISSGGSYTYYDDTVTG

SEQ ID NO: 68 - DNA, 51 bp, Homo sapiens
aaaattagtagtggtgggagttacacctactatgatgacactgtgacgggc

SEQ ID NO: 69 - protein, 17 aa, Homo sapiens
KISSGGSYTYYSDTVTG

SEQ ID NO: 70 - DNA, 51 bp, Homo sapiens
aaaattagtagtggtgggagttacacctactattctgacactgtgacgggc

SEQ ID NO: 71 - protein, 17 aa, Homo sapiens
KISSGGSYTYYPDTVTP

SEQ ID NO: 72 - DNA, 51 bp, Homo sapiens
aaaattagtagtggtgggagttacacctactatcctgacactgtgacgccg

SEQ ID NO: 73 - protein, 17 aa, Homo sapiens
KISSGGSYTYYPDTDTG

SEQ ID NO: 74 - DNA, 51 bp, Homo sapiens
aaaattagtagtggtgggagttacacctactatcctgacactgatacgggc

SEQ ID NO: 75 - protein, 17 aa, Homo sapiens
KISPGGSYTYYSDTVTG

SEQ ID NO: 76 - DNA, 51 bp, Homo sapiens
aaaattagtccgggtgggagttacacctactattctgacactgtgacgggc

SEQ ID NO: 77 - protein, 17 aa, Homo sapiens
KISPGGSWTYYDDTVTG

SEQ ID NO: 78 - DNA, 51 bp, Homo sapiens
aaaattagtccgggtgggagttggacctactatgatgacactgtgacgggc

SEQ ID NO: 79 - protein, 10 aa, Homo sapiens
QLWGSYALDY

SEQ ID NO: 80 - DNA, 30 bp, Homo sapiens
cagttatgggggtcgtatgctcttgactac

SEQ ID NO: 81 - protein, 10 aa, Homo sapiens
QLWGYYALDT

SEQ ID NO: 82 - DNA, 30 bp, Homo sapiens
cagttatgggggtactatgctcttgacacg

SEQ ID NO: 83 - protein, 10 aa, Homo sapiens
QLWGTYALDY

SEQ ID NO: 84 - DNA, 30 bp, Homo sapiens
cagttatgggggacttatgctcttgactac

SEQ ID NO: 85 - protein, 10 aa, Homo sapiens
QLWGNYALDY

SEQ ID NO: 86 - DNA, 30 bp, Homo sapiens
cagttatgggggaattatgctcttgactac

SEQ ID NO: 87 - protein, 10 aa, Homo sapiens
QLWGYYALDF

SEQ ID NO: 88 - DNA, 30 bp, Homo sapiens
cagttatgggggtactatgctcttgacttt

SEQ ID NO: 89 - protein, 10 aa, Homo sapiens
QLWGYYALDI

SEQ ID NO: 90 - DNA, 30 bp, Homo sapiens
cagttatgggggtactatgctcttgacatt

SEQ ID NO: 91 - protein, 10 aa, Homo sapiens
QLWGYYALDY

SEQ ID NO: 92 - DNA, 30 bp, Homo sapiens
cagttatgggggtactatgctcttgactac

SEQ ID NO: 93 - protein, 106 aa, Homo sapiens
EIVLTQSPATLSLSPGERATLSCSASSSVSYMYWYQQKPGQAPRLLIYDTSNLASGIPARFSGSGSGTDFTLTISSLEPEDFAVYYCSQWSGYPYTFGGGTKVEIK

SEQ ID NO: 94 - DNA, 318 bp, Homo sapiens
gaaattgtgttgacacagtctccagccaccctgtctttgtctccaggggaaagagccaccctctcctgcagtgccagctcaagtgtaagttacatgtactggtaccaacagaaacctggccaggctcccaggctcctcatctatgacacatccaacctggcttctggcatcccagccaggttcagtggcagtgggtctgggacagacttcactctcaccatcagcagcctagagcctgaagattttgcagtttattactgttctcagtggagtggttacccatacacgttcggcggagggaccaaggtggagatcaaa

SEQ ID NO: 95 - protein, 119 aa, Homo sapiens
EVQLVESGGGLVQPGGSLRLSCAASGFTFSSFAMSWVRQAPGKGLEWVAKISSGGSYTYYPDTVTGRFTISRDNAKNSLYLQMNSLRAEDTAVYYCARQLWGYYALDYWGQGTTVTVSS

SEQ ID NO: 96 - DNA, 357 bp, Homo sapiens
gaggtgcagctggtggagtctgggggaggcttggtccagcctggggggtccctgagactctcctgtgcagcctctggattcacctttagtagctttgccatgtcttgggtccgccaggctccagggaaggggctggagtgggtggccaaaattagtagtggtgggagttacacctactatcctgacactgtgacgggccgattcaccatctccagagacaacgccaagaactcactgtatctgcaaatgaacagcctgagagccgaggacacggctgtgtattactgtgcgagacagttatgggggtactatgctcttgactactggggccaagggaccacggtcaccgtctcctca

SEQ ID NO: 97 - protein, 106 aa, Homo sapiens
EIVLTQSPATLSLSPGERATLSCSASISVSYMYWYQQKPGQAPRLLIYDMSNLASGIPARFSGSGSGTDFTLTISSLEPEDFAVYYCMQWSGYPYTFGGGTKVEIK

SEQ ID NO: 98 - DNA, 318 bp, Homo sapiens
gaaattgtgttgacacagtctccagccaccctgtctttgtctccaggggaaagagccaccctctcctgcagtgccagcattagtgtaagttacatgtactggtaccaacagaaacctggccaggctcccaggctcctcatctatgacatgtccaacctggcttctggcatcccagccaggttcagtggcagtgggtctgggacagacttcactctcaccatcagcagcctagagcctgaagattttgcagtttattactgtatgcagtggagtggttacccatacacgttcggcggagggaccaaggtggagatcaaa

SEQ ID NO: 99 - protein, 119 aa, Homo sapiens
EVQLVESGGGLVQPGGSLRLSCAASGFTFSPFAMSWVRQAPGKGLEWVAKISPGGSWTYYSDTVTGRFTISRDNAKNSLYLQMNSLRAEDTAVYYCARQLWGYYALDIWGQGTTVTVSS

SEQ ID NO: 100 - DNA, 357 bp, Homo sapiens
gaggtgcagctggtggagtctgggggaggcttggtccagcctggggggtccctgagactctcctgtgcagcctctggattcacctttagtccttttgccatgtcttgggtccgccaggctccagggaaggggctggagtgggtggccaaaattagtccgggtgggagttggacctactattctgacactgtgacgggccgattcaccatctccagagacaacgccaagaactcactgtatctgcaaatgaacagcctgagagccgaggacacggctgtgtattactgtgcgagacagttatgggggtactatgctcttgacatttggggccaagggaccacggtcaccgtctcctca

SEQ ID NO: 101 - protein, 106 aa, Homo sapiens
EIVLTQSPATLSLSPGERATLSCSASYSVSYMYWYQQKPGQAPRLLIYDMSNLASGIPARFSGSGSGTDFTLTISSLEPEDFAVYYCMQWSGYPYTFGGGTKVEIK

SEQ ID NO: 102 - DNA, 318 bp, Homo sapiens
gaaattgtgttgacacagtctccagccaccctgtctttgtctccaggggaaagagccaccctctcctgcagtgccagctatagtgtaagttacatgtactggtaccaacagaaacctggccaggctcccaggctcctcatctatgacatgtccaacctggcttctggcatcccagccaggttcagtggcagtgggtctgggacagacttcactctcaccatcagcagcctagagcctgaagattttgcagtttattactgtatgcagtggagtggttacccatacacgttcggcggagggaccaaggtggagatcaaa

SEQ ID NO: 103 - protein, 119 aa, Homo sapiens
EVQLVESGGGLVQPGGSLRLSCAASGFQFSSFAMSWVRQAPGKGLEWVAKISPGGSYTYYSDTVTGRFTISRDNAKNSLYLQMNSLRAEDTAVYYCARQLWGYYALDFWGQGTTVTVSS

SEQ ID NO: 104 - DNA, 357 bp, Homo sapiens
gaggtgcagctggtggagtctgggggaggcttggtccagcctggggggtccctgagactctcctgtgcagcctctggattccagtttagtagctttgccatgtcttgggtccgccaggctccagggaaggggctggagtgggtggccaaaattagtccgggtgggagttacacctactattctgacactgtgacgggccgattcaccatctccagagacaacgccaagaactcactgtatctgcaaatgaacagcctgagagccgaggacacggctgtgtattactgtgcgagacagttatgggggtactatgctcttgacttttggggccaagggaccacggtcaccgtctcctca

SEQ ID NO: 105 - protein, 23 aa, Homo sapiens
EIVLTQSPATLSLSPGERATLSC

SEQ ID NO: 106 - protein, 15 aa, Homo sapiens
WYQQKPGQAPRLLIY

SEQ ID NO: 107 - protein, 32 aa, Homo sapiens
GIPARFSGSGSGTDFTLTISSLEPEDFAVYYC

SEQ ID NO: 108 - protein, 10 aa, Homo sapiens
FGGGTKVEIK

SEQ ID NO: 109 - protein, 25 aa, Homo sapiens [sequence omitted]

SEQ ID NO: 110 - protein, 14 aa, Homo sapiens
WVRQAPGKGLEWVA

SEQ ID NO: 111 - protein, 32 aa, Homo sapiens
RFTISRDNAKNSLYLQMNSLRAEDTAVYYCAR

SEQ ID NO: 112 - protein, 11 aa, Homo sapiens
WGQGTTVTVSS

SEQ ID NO: 113 - protein, 17 aa, Homo sapiens
EISSGGSYTYYPDTVTG

SEQ ID NO: 114 - protein, 10 aa, Homo sapiens
GLWGYYALDY

SEQ ID NO: 115 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 116 - protein, 106 aa, Homo sapiens; identical to SEQ ID NO: 93

SEQ ID NO: 117 - DNA, 378 bp, Homo sapiens
atggaagccccagcgcagcttctcttcctcctgctactctggctcccagataccaccggagaaattgtgttgacacagtctccagccaccctgtctttgtctccaggggaaagagccaccctctcctgcagtgccagctcaagtgtaagttacatgtactggtaccaacagaaacctggccaggctcccaggctcctcatctatgacacatccaacctggcttctggcatcccagccaggttcagtggcagtgggtctgggacagacttcactctcaccatcagcagcctagagcctgaagattttgcagtttattactgttctcagtggagtggttacccatacacgttcggcggagggaccaaggtggagatcaaa

SEQ ID NO: 118 - protein, 119 aa, Homo sapiens
EVQLVESGGGLVQPGGSLRLSCAASGFTFSSFAMSWVRQAPGKGLEWVAKISPGGSYTYYPDTVTGRFTISRDNAKNSLYLQMNSLRAEDTAVYYCARQLWGYYALDYWGQGTTVTVSS

SEQ ID NO: 119 - DNA, 414 bp, Homo sapiens
atggagtttggcctgagctgggttttccttgttgctattttagaaggtgtccagtgtgaggtgcagctggtggagtctgggggaggcttggtccagcctggggggtccctgagactctcctgtgcagcctctggattcacctttagtagctttgccatgtcttgggtccgccaggctccagggaaggggctggagtgggtggccaaaattagtcccggtgggagttacacctactatcctgacactgtgacgggccgattcaccatctccagagacaacgccaagaactcactgtatctgcaaatgaacagcctgagagccgaggacacggctgtgtattactgtgcgagacagttatgggggtactatgctcttgactactggggccaagggaccacggtcaccgtctcctca

SEQ ID NO: 120 - protein, 106 aa, Homo sapiens
EIVLTQSPATLSLSPGERATLSCSASSSVSYMYWYQQKPGQAPRLLIYDFSNLASGIPARFSGSGSGTDFTLTISSLEPEDFAVYYCMQWSGYPYTFGGGTKVEIK

SEQ ID NO: 121 - DNA, 378 bp, Homo sapiens
atggaagccccagcgcagcttctcttcctcctgctactctggctcccagataccaccggagaaattgtgttgacacagtctccagccaccctgtctttgtctccaggggaaagagccaccctctcctgcagtgccagctcaagtgtaagttacatgtactggtaccaacagaaacctggccaggctcccaggctcctcatctatgacttctccaacctggcttctggcatcccagccaggttcagtggcagtgggtctgggacagacttcactctcaccatcagcagcctagagcctgaagattttgcagtttattactgtatgcagtggagtggttacccatacacgttcggcggagggaccaaggtggagatcaaa

SEQ ID NO: 122 - protein, 119 aa, Homo sapiens
EVQLVESGGGLVQPGGSLRLSCAASGFQFSPFAMSWVRQAPGKGLEWVAKISPGGSWTYYSDTVTGRFTISRDNAKNSLYLQMNSLRAEDTAVYYCARQLWGYYALDIWGQGTTVTVSS

SEQ ID NO: 123 - DNA, 414 bp, Homo sapiens
atggagtttggcctgagctgggttttccttgttgctattttagaaggtgtccagtgtgaggtgcagctggtggagtctgggggaggcttggtccagcctggggggtccctgagactctcctgtgcagcctctggattccagtttagtccctttgccatgtcttgggtccgccaggctccagggaaggggctggagtgggtggccaaaattagtcccggtgggagttggacctactatagcgacactgtgacgggccgattcaccatctccagagacaacgccaagaactcactgtatctgcaaatgaacagcctgagagccgaggacacggctgtgtattactgtgcgagacagttatgggggtactatgctcttgacatttggggccaagggaccacggtcaccgtctcctca

SEQ ID NO: 124 - protein, 106 aa, Homo sapiens; identical to SEQ ID NO: 97

SEQ ID NO: 125 - DNA, 378 bp, Homo sapiens
atggaagccccagcgcagcttctcttcctcctgctactctggctcccagataccaccggagaaattgtgttgacacagtctccagccaccctgtctttgtctccaggggaaagagccaccctctcctgcagtgccagcattagtgtaagttacatgtactggtaccaacagaaacctggccaggctcccaggctcctcatctatgacatgtccaacctggcttctggcatcccagccaggttcagtggcagtgggtctgggacagacttcactctcaccatcagcagcctagagcctgaagattttgcagtttattactgtatgcagtggagtggttacccatacacgttcggcggagggaccaaggtggagatcaaa

SEQ ID NO: 126 - protein, 119 aa, Homo sapiens
EVQLVESGGGLVQPGGSLRLSCAASGFQFSSFAMSWVRQAPGKGLEWVAKISPGGSYTYYSDTVTGRFTISRDNAKNSLYLQMNSLRAEDTAVYYCARQLWGYYALDIWGQGTTVTVSS

SEQ ID NO: 127 - DNA, 414 bp, Homo sapiens
atggagtttggcctgagctgggttttccttgttgctattttagaaggtgtccagtgtgaggtgcagctggtggagtctgggggaggcttggtccagcctggggggtccctgagactctcctgtgcagcctctggattccagtttagtagctttgccatgtcttgggtccgccaggctccagggaaggggctggagtgggtggccaaaattagtcccggtgggagttacacctactatagcgacactgtgacgggccgattcaccatctccagagacaacgccaagaactcactgtatctgcaaatgaacagcctgagagccgaggacacggctgtgtattactgtgcgagacagttatgggggtactatgctcttgacatttggggccaagggaccacggtcaccgtctcctca

SEQ ID NO: 128 - protein, 106 aa, Homo sapiens
EIVLTQSPATLSLSPGERATLSCSASYSVSYMYWYQQKPGQAPRLLIYDFSNLASGIPARFSGSGSGTDFTLTISSLEPEDFAVYYCMQWSGYPYTFGGGTKVEIK

SEQ ID NO: 129 - DNA, 378 bp, Homo sapiens
atggaagccccagcgcagcttctcttcctcctgctactctggctcccagataccaccggagaaattgtgttgacacagtctccagccaccctgtctttgtctccaggggaaagagccaccctctcctgcagtgccagctacagtgtaagttacatgtactggtaccaacagaaacctggccaggctcccaggctcctcatctatgacttctccaacctggcttctggcatcccagccaggttcagtggcagtgggtctgggacagacttcactctcaccatcagcagcctagagcctgaagattttgcagtttattactgtatgcagtggagtggttacccatacacgttcggcggagggaccaaggtggagatcaaa

SEQ ID NO: 130 - protein, 119 aa, Homo sapiens
EVQLVESGGGLVQPGGSLRLSCAASGFQFSPFAMSWVRQAPGKGLEWVAKISPGGSWTYYPDTDTGRFTISRDNAKNSLYLQMNSLRAEDTAVYYCARQLWGYYALDFWGQGTTVTVSS

SEQ ID NO: 131 - DNA, 414 bp, Homo sapiens
atggagtttggcctgagctgggttttccttgttgctattttagaaggtgtccagtgtgaggtgcagctggtggagtctgggggaggcttggtccagcctggggggtccctgagactctcctgtgcagcctctggattccagtttagtccctttgccatgtcttgggtccgccaggctccagggaaggggctggagtgggtggccaaaattagtcccggtgggagttggacctactatcctgacactgacacgggccgattcaccatctccagagacaacgccaagaactcactgtatctgcaaatgaacagcctgagagccgaggacacggctgtgtattactgtgcgagacagttatgggggtactatgctcttgacttctggggccaagggaccacggtcaccgtctcctca

SEQ ID NO: 132 - protein, 10 aa, Homo sapiens
  unsure (2)..(3), (4)..(5), (7): Wherein Xaa can be any one of the twenty naturally occurring amino acids.
SXXXXVXYMY

SEQ ID NO: 133 - protein, 7 aa, Homo sapiens
  unsure (2),(4),(6): Wherein Xaa can be any one of the twenty naturally occurring amino acids.
DXSXLXS

SEQ ID NO: 134 - protein, 9 aa, Homo sapiens
  unsure (1)..(2): Wherein Xaa can be any one of the twenty naturally occurring amino acids.
XXWSGYPYT

SEQ ID NO: 135 - protein, 10 aa, Homo sapiens
  unsure (3)..(4),(6),(9): Wherein Xaa can be any one of the twenty naturally occurring amino acids.
GFXXSXFAXS

SEQ ID NO: 136 - protein, 17 aa, Homo sapiens
  unsure (2),(4),(8)..(9),(11)..(12),(15)..(17): Wherein Xaa can be any one of the twenty naturally occurring amino acids.
KXSXGGSXXYXXDTXXX

SEQ ID NO: 137 - protein, 10 aa, Homo sapiens
  unsure (5),(10): Wherein Xaa can be any one of the twenty naturally occurring amino acids.
QLWGXYALDX

SEQ ID NO: 138 - protein, 9 aa, Homo sapiens
QQWSGYPYT

SEQ ID NO: 139 - protein, 119 aa, Mus musculus [sequence omitted]

SEQ ID NO: 140 - protein, 106 aa, Mus musculus [sequence omitted]

SEQ ID NO: 141 - protein, 5 aa, Mus musculus
SFAMS

SEQ ID NO: 142 - protein, 17 aa, Mus musculus
EISSGGSYTYYPDTVTG

SEQ ID NO: 143 - protein, 10 aa, Mus musculus
GLWGYYALDY

SEQ ID NO: 144 - protein, 10 aa, Mus musculus
SASSSVSYMY

SEQ ID NO: 145 - protein, 7 aa, Mus musculus
DTSNLAS

SEQ ID NO: 146 - protein, 9 aa, Mus musculus
QQWSGYPYT